(19) United States
(12) Patent Application Publication
(10) Pub. No.: US 2003/0224942 A1
(43) Pub. Date: Dec. 4, 2003

(54) Alkylamino-1,3,5-triazines, processes for their preparation and their use as herbicides and plant growth regulators

(76) Inventors: Hartmut Ahrens, Frankfurt (DE); Hansjorg Dietrich, Hofheim (DE); Lothar Willms, Hofheim (DE); Hubert Menne, Hofheim (DE); Hermann Bieringer, Eppstein (DE); Thomas Auler, Bad Soden (DE)

Correspondence Address: FROMMER LAWRENCE & HAUG, 745 FIFTH AVENUE- 10TH FL., NEW YORK, NY, 10151, US

(21) Appl. No.: 10/404,307

(22) Filed: Apr. 1, 2003

Related U.S. Application Data
(63) Continuation of application No. 10/116,831, filed on Apr. 5, 2002, now abandoned.

(30) Foreign Application Priority Data
Apr. 9, 2001 (DE) 101 17 707.0

(51) Int. Cl. A01N 43/68; C07D 251/48; C 07D 4 3/02

(52) U.S. Cl. 504/232; 544/208; 544/209

(57) ABSTRACT

2,4-Diamino-1,3,5-triazines, process for their preparation and their use as herbicides and plant growth regulators,

Compounds of the formula (I) and salts thereof

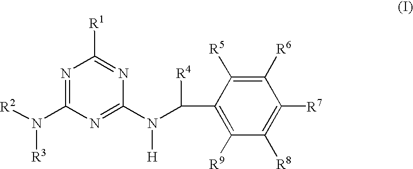

where R¹ is (C₁-C₁₀)alkyl, (C₃-C₆)cycloalkyl, (C₁-C₁₀)alkoxy or (C₁-C₁₀)alkylthio, optionally halogenated and in the case of cyclic radicals optionally also substituted by (C₁-C₆)alkyl and (C₁-C₆)haloalkyl; R² and R³ are H, (C₁-C₄)alkyl, CHO or [(C₁-C₁₀)alkyl]carbonyl, optionally halogenated; R⁴ is hydrogen, (C₁-C₁₀)alkyl, (C₂-C₁₀)alkenyl, (C₂-C₁₀)alkynyl or (C₃-C₆)cycloalkyl, where each of the 4 last-mentioned radicals is unsubstituted or halogenated and in the case of cyclic radicals optionally substituted by (C₁-C₆)alkyl and (C₁-C₆)haloalkyl; R⁴ is hydrogen, (C₁-C₁₀)alkyl, (C₂-C₁₀)alkenyl, (C₂-C₁₀)alkynyl or (C₃-C₆)cycloalkyl, where each of the 4 last-mentioned radicals is unsubstituted or substituted by one or more radicals selected from the group consisting of halogen and in the case of cyclic radicals also (C₁-C₆)alkyl and (C₁-C₆)haloalkyl;

each of the radicals R⁵, R⁶, R⁷, R⁸ and R⁹ independently of the others is hydrogen, halogen; nitro. cyano, thiocyanato, (C₁-C₁₀)alkyl, (C₂-C₁₀)alkenyl, (C₂-C₁₀)alkynyl, (C₁-C₁₀)alkoxy, (C₂-C₁₀)alkenyloxy, (C₂-C₁₀)alkynyloxy, (C₁-C₁₀)alkylthio, (C₂-C₁₀)alkenylthio, (C₂-C₁₀)alkynylthio, (C₃-C₆)cycloalkyl, (C₅-C₆)cycloalkenyl, phenyl or heterocyclyl, where each of the 13 last-mentioned radicals is unsubstituted or substituted, where at least one of the radicals from the group consisting of R⁵, R⁶, R⁷, R⁸ and R⁹ is different from hydrogen and where at least one of the radicals R⁵, R⁶, R⁷, R⁸ and R⁹ is selected from the group consisting of the radicals (C₂-C₁₀)alkenyl, (C₂-C₁₀)alkynyl, (C₃-C₆)cycloalkyl and (C₅-C₆)cycloalkenyl, where each of the 4 last-mentioned radicals is unsubstituted or substituted, are suitable for use as herbicides and plant growth regulators. The compounds (I) can be prepared by the process claimed in claim 6.

The invention relates to the technical field of the crop protection agents, such as herbicides and plant growth regulators, in particular herbicides for the selective control of harmful plants in crops of useful plants.

It is known that 4-amino-2-[N-(1-arylalkyl)amino]-1,3,5-triazines which are optionally substituted in the 6-position on the triazine ring by unsubstituted or halogenated hydrocarbon groups or else by other groups, and their derivatives, have herbicidal and plant-growth-regulating properties; cf., for example, DE-A-19826670, DE-A-19828519, EP-A-0191496, EP-A-573897, EP-A-573898, JP-A-62,294,669, JP-A-62,298,577, JP-A-1 0025211, JP-A-08198712, US-A-3816419, US-A-5290754 (WO-A-90/9378, EP-A-0411153), WO-A-95/06642, WO-A-97/08156, WO-A-97/31904, WO-A-97/35481, WO-A-98/10654, WO-A-98/15536, WO-A-98/15537, WO-A-98/15538, WO-A-98/15539, WO-A-98/34925, WO-A-98/42684, WO-A-99/188100, WO-A-99/19309, WO-A-99/37627, WO-A-99/44999, WO-A-99/46249, WO-A-99/65882, WO-A-00/00480, WO-A-00/1 6627, WO-A-00/32580, WO-A-00/47579, WO-A-00/56722, WO-A-00/69854 and WO-A-01/10849 and the literature cited in these publications.

Some of the known active compounds have, when used, disadvantages, such as insufficient herbicidal activity against harmful plants, too limited a spectrum of harmful plants which can be controlled with an active compound, or insufficient selectivity in crops of useful plants. Other active compounds cannot be prepared economically on an industrial scale, owing to reagents and precursors which are difficult to obtain, or they have insufficient chemical stability. Accordingly, it is desirable to provide alternative active compounds which may optionally be employed advantageously as herbicides or plant growth regulators. The present invention provides compounds of the formula (I) and salts thereof

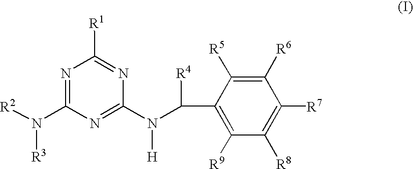

where

R¹ is (C₁-C₁₀)alkyl, (C₃-C₆)cycloalkyl, (C₁-C₁₀)alkoxy or (C₁-C₁₀)alkylthio, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl,

preferably R¹=(C₁-C₁₀)alkyl or (C₃-C₆)cycloalkyl, where each of the 2 last-mentioned radicals is unsubstituted or substituted by one or more radicals selected from the group consisting of halogen and, in the case of cyclic radicals, also (C₁-C₆)alkyl and (C₁-C₆)haloalkyl,

R² and R³ independently of one another are hydrogen, (C₁-C₄)alkyl, formyl or [(C₁-C₁₀)alkyl]carbonyl which is unsubstituted or substituted by one or more halogen atoms,

R⁴ is hydrogen, (C₁-Clo)alkyl, (C₂-Clo)alkenyl, (C₂-C₁₀)alkynyl or (C₃-C₆)cycloalkyl, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl,

each of the radicals R⁵, R⁶, R⁷, R⁸ and R⁹ independently of the others is hydrogen, halogen, nitro, cyano, thiocyanato, (C₁-C₁₀)alkyl, (C₂-C₁₀)alkenyl, (C₂-C₁₀)alkynyl, (C₁-C₁₀)alkoxy, (C₂-C₁₀)alkenyloxy, (C₂-C₁₀)alkynyloxy, (C₁-C₁₀)alkylthio, (C₂-C₁₀)alkenylthio, (C₂-C₁₀)alkynylthio, (C₃-C₆)cycloalkyl, (C₅-C₆)cycloalkenyl, phenyl or heterocyclyl, where each of the 13 last-mentioned radicals is unsubstituted or substituted,

preferably unsubstituted or substituted by one or more radicals selected from the group consisting of halogen, cyano, hydroxyl, amino, (C₁-C₆)alkoxy, (C₁-C₆)haloalkoxy, and (C₁-C₆)alkylthio and, in the case of cyclic radcials, also (C₁-C₆)alkyl and (C₁-C₆)haloalkyl, in particular unsubstituted or substituted by one or more radicals selected from the group consisting of halogen, cyano, hydroxyl and amino and, in the case of cyclic radicals, also (C₁-C₆)alkyl, (C₁-C₆)haloalkyl, (C₁-C₆)alkoxy, (C₁-C₆)haloalkoxy and (C₁-C₆)alkylthio,

where at least one of the radicals, preferably at least two radicals, in particular 2 or 3 radicals, especially 2 radicals, from the group consisting of R⁵, R⁶, R⁷, R⁸ and R⁹ is different from hydrogen and where at least one of the radicals R⁵, R⁶, R⁷, R⁸ and R⁹, preferably one of the radicals mentioned, is selected from the group consisting of the radicals (C₂-C₁₀)alkenyl, (C₂-C₁₀)alkynyl, (C₃-C₆)cycloalkyl and (C₅-C₆)cycloalkenyl, where each of the 4 last-mentioned radicals is unsubstituted or substituted, preferably unsubstituted or substituted by one or more radicals selected from the group consisting of halogen, cyano, hydroxy and amino and, in the case of cyclic radicals, also (C₁-C₆)alkyl, (C₁-C₆)haloalkyl, (C₁-C₆)alkoxy, (C₁-C₆)haloalkoxy and (C₁-C₆)alkylthio.

The invention also provides all stereoisomers embraced by the formula (I), and their mixtures. Such compounds of the formula (I) contain one or more asymmetrically substituted carbon atoms or else double bonds which are not specifically mentioned in the general formulae (I). The possible stereoisomers defined by their specific spatial form, such as enantiomers and diastereomers, including Z and E isomers, are all embraced by the formula (I) and can be obtained by customary methods from mixtures of the stereoisomers or else by stereoselective reactions in combination with the use of stereochemically pure or enriched starting materials. Of particular interest are also the stereoisomers which are formed by the asymmetrically substituted carbon atom to which the group R⁴ is attached if R⁴ is not hydrogen. The isomers which are R- and S-configured at this center, which are enantiomers, if the molecule of the formula (I) contains no further asymmetrically substituted carbon atom, thus also form part of the subject matter of the invention. In general, the biological activities of these R and S isomers are not identical, but in the individual case, one of the isomers has, depending on the species of harmful plant and the crop, a higher herbicidal action or selectivity.

If compounds of the formula (I) are also capable of forming tautomers which are structurally not formally represented or embraced by the formula (I), these tautomers are nevertheless embraced by the definition of the compounds of the formula (I) according to the invention.

The compounds of the formula (I) can form salts by addition of a suitable inorganic or organic acid, such as, for example, HCI, HBr, H₂SO₄ or HNO₃, but also oxalic acid or sulfonic acids, to a basic group, such as, for example, amino or alkylamino. Suitable substituents which are present in deprotonated form, such as, for example, sulfonic acids or carboxylic acids, can form inner salts with groups which for their part can be protonated, such as amino groups. Salts can also be formed by replacing the hydrogen of suitable substituents, such as, for example, sulfonlic acid s or carboxylic acids, by an agriculturally suitable cation. These salts are, for example, metal salts, in particular alkali metal salts or alkaline earth metal salts, in particular sodium salts and potassium salts, or else ammonium salts or salts with organic amines.

In the formula (I) and all the formulae hereinbelow, the radicals alkyl, alkoxy, haloalkyl, haloalkoxy, alkylamino and alkylthio and the corresponding unsaturated and/or substituted radicals can in each case be straight-chain or branched in the carbon skeleton. Unless specifically mentioned otherwise, the lower carbon skeletons, for example with 1 to 6 carbon atoms, or in the case of unsaturated groups with 2 to 6 carbon atoms, are preferred for these radicals. Alkyl radicals, also in the composed meanings, such as alkoxy, haloalkyl, and the like, are, for example, methyl, ethyl, n- or isopropyl, n-, i-, t- or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the meaning of the possible unsaturated radicals which correspond to the alkyl radicals; alkenyl is, for example, allyl, 1-methylprop-2-en-1-yl, 2-methylprop-2-en-1-yl, but-2-en-1-yl, but-3-en-1-yl, 1-methyl-but-3-en-1-yl and 1-methyl-but-2-en-1-yl; alkynyl is, for example, propargyl, but-2-yn-1-yl, but-3-yn-1-yl, 1-methylbut-3-yn-1-yl; alkenyl also includes in particular straight-chain or branched hydrocarbons with more than one-double bond, such as 1,3-butadienyl and 1,4-pentadienyl, but also allenyl or aminlenyl radicals with one or more cumulated double bonds, such as, for example, allenyl (1,2-propadienyl), 1,2-butadienyl and 1 ,2,3-pentatrienyl; alkynyl is, for example, propargyl, but-2-yn-1-yl, but-3-yn-1-yl, 1-methylbut-3-yn-1-1 yl. Alkynyl also includes in particular straight-chain or branched hydrocarbon radicals with more than one triple bond or also with one or more triple bonds and one or more double bonds, such as, for example, 1,3-butatrienyl or 3-pentene-1-yn-1-yl.

Alkylidene, for example also in the form (C₁-C₁₀)alkylidene, is the radical of a straight-chain or branched alkane which is attached via a double bond, the position of the binding site not yet being fixed. In the case of a branched alkane, the only possible positions are, of course, those where two hydrogen atoms can be replaced by the double bond; examples of radicals are ═CH₂, ′CH—CH₃, ═C(CH₃)—CH₃, ═C(CH₃)—C₂H₅ or ═C(C₂H₅)—C₂H₅.

Cycloalkyl is a carbocyclic saturated ring system having preferably 3-8 carbon atoms, for example cyclopropyl, cyclobutyl, cyclopentyl or cyclohexyl. In the case of substituted cycloalkyl, this includes cyclic systems with substituents, where the substitutents are attached to the cycloalkyl radical via a double bond, for example an alkylidene group such as methylidene. Substituted cycloalkyl also includes polycyclic aliphatic systems, such as, for example, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl.

Cycloalkenyl is a carbocyclic, non-aromatic, partially unsaturated ring system having preferably 4-8 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. For substituted cycloalkenyl, the illustrations for substituted cycloalkyl apply correspondingly.

Halogen is, for example, fluorine, chlorine, bromine or iodine. Haloalkyl, -alkenyl and -alkynyl is alkyl, alkenyl and alkynyl, respectively, which is partially or fully substituted by halogen, preferably by fluorine, chlorine and/or bromine, in particular by fluorine or chlorine, for example, monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl) can be saturated, unsaturated or heteroaromatic; unless defined otherwise, it preferably contains one or more, in particular 1, 2 or 3, heteroatoms in the heterocyclic ring, preferably selected from the group consisting of N, O and S; it is preferably an aliphatic heterocyclyl radical having 3 to 7 ring atoms or a heteroaromatic radical having 5 or 6 ring atoms. The heterocyclic radical can, for example, be a heteroaromatic radical or ring (heteroaryl), such as, for example, a mono-, bi- or polycyclic aromatic system, in which at least 1 ring contains one or more heteroatoms. It is preferably a heteroaromatic ring having one heteroatom selected from the group consisting of N, 0 and S, for example pyridyl, pyrrolyl, thienyl or furyl; furthermore, preferably, it is a corresponding heteroaromatic ring having 2 or 3 heteroatoms, for example pyrimidinyl, pyridazinyl, pyrazinyl, triazinyl, thiazolyl, thiadiazolyl, oxazolyl, isoxazolyl, pyrazolyl, imidazolyl and triazolyl. Furthermore preferably, it is a partially or fully hydrogenated heterocyclic radical having one heteroatom selected from the group consisting of N, O and S, for example oxiranyl, oxetanyl, oxoianyl (=tetrahydrofuryl), oxanyl, pyrrolidyl or piperidyl.

Furthermore preferably, it is a partially or fully hydrogenated heterocyclic radical having 2 heteroatoms selected from the group consisting of N, 0 and S, for example piperazinyl, dioxolanyl, oxazolinyl, isoxazolinyl, oxazolidinyl, isoxazolidinyl and morpholinyl.

Possible substituents for a substituted heterocyclic radical are the substituents mentioned further below, and additionally also oxo. The oxo group can also be present at the hetero ring atoms which can exist in different oxidation states, for example at N and S.

Substituted radicals, such as a substituted alkyl, alkenyl, alkynyl, aryli, phenyl, benzyl, heterocyclyl and heteroaryl radical, are, for example, a substituted radical derived from the unsubstituted skeleton, where the substituents are, for example, one or more, preferably 1, 2 or 3, radicals selected from the group consisting of halogen, alkoxy, haloalkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino, such as acylamino, mono- and dialkylamino, and alkylsulfinyl, haloalkylsulfinyl, alkylsulfonyl, haloalkylsulfonyl and, in the case of cyclic radicals, also alkyl, haloalkyl alkylthioalkyl, alkoxyalkyl, substituted or unsubstituted mono- and dialkylaminoalkyl and hydroxyalkyl; the term “substituted radicals”, such as in substituted alkyl and the like, includes as substituents, in addition to the saturated hydrocarbon-containing radicals mentioned, the corresponding unsaturated aliphatic and aromatic radicals, such as unsubstituted or substituted alkenyl, alkynyl, alkenyloxy, alkynyloxy, phenyl, phenoxy etc. Substituted cyclic radicals having aliphatic moieties in the ring also include cyclic systems having substituents which are attached to the ring via a double bond, for example those substittuted by an alkylidene group, such as methylidene or ethylidene.

Among the radicals with carbon atoms, preference is given to those having 1 to 4 carbon atoms, in particular 1 or 2 carbon atoms. In general, preference is given to substituents selected from the group consisting of halogen, for example fluorine and chlorine, (C₁-C₄)alkyl, preferably methyl or ethyl, (C₁-C₄)haloalkyl, preferably trifluorornethyl, (C₁-C₄)alkoxy, preferably methoxy or ethoxy, (C₁-C₄)haloalkoxy, nitro and cyano. Particular preference is given here to the substituents methyl, methoxy, fluorine and chlorine.

Substituted amino, such as mono- or disubstituted amino, is a radical from the group of the substituted amino radicals which are N-substituted, for example, by one or two identical or different radicals selected from the group consisting of alkyl, alkoxy, acyl and aryl; preferably mono- and dialkylamino, mono- and diarlamino, acylamino,

N-alkyl-N-arylamino, N-alkyl-N-acylamino and N-heterocycles; preference is given to alkyl radicals having 1 to 4 carbon atoms; aryl is preferably phenyl or substituted phenyl; for acyl, the definition mentioned further below applies, preferably (C₁-C₄)alkanoyl. This applies correspondingly to substituted hydroxylamino or hydrazino.

Unsubstituted or substituted phenyl is preferably phenyl which is unsubstituted or mono- or polysubstituted, preferably up to trisubstituted, by identical or different radicals selected from the group consisting of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyls, 2-, 3- and 4-chlorophenyl, 2-, 3- and 4-trifluoromethylphenyl and 2-,3-, and 4- trichloromethylphenyl, 2,4-, 3,5-, 2,5- and 2,3-dichlorophenyl, o-, m- and p-methoxyphenyl.

Acyl is a radical of an organic acid which is formally formed by removing a hydroxyl group from the acid function, where the organic radical in the acid can also be attached to the acid function via a heteroatom. Examples of acyl are the radical —CO—R of a carboxylic acid HO—CO—R and radicals of acids derived therefrom, such as thiocarbonic acid, unsubstituted or N-substituted iminocarboxylic acids or the radical of carbonic monoesters, N-substituted carbamic acid, sulfonic acids, sulfinic acids, N-substituted sulfonamide acids, phosphonic acids, phosphinic acids. Acyl is, for example, formyl, alkylcarbonyl, such as [(C₁-C₄)alkyl]carbonyl, phenylcarbonyl, alkyloxycarbonyl, phenyloxycarbonyl, benzyloxycarbonyl, alkylsulfonyl, alkylsulfinyl, N-alkyl-1-iminoalkyl and other radicals of organic acids. The radicals can in each case be further substituted in the alkyl or phenyl moiety, for example in the alkyl moiety by one or more radicals selected from the group consisting of halogen, alkoxy, phenyl and phenoxy; examples of substituents in the phenyl moiety are the substituents which have already been mentioned further above generally for substituted phenyl.

Acyl is preferably an acyl radical in the more restricted sense, i.e. a radical of an organic acid where the acid group is directly attached to the carbon atom of an organic radical, for example formyl, alkylcarbonyl, such as acetyl or [(C₁-C₄)alkyl]carbonyl, phenylcarbonyl, alkyisulfonyl, alkylsulfinyl and other radicals of organic acids.

In particular for reasons of better herbicidal activity, better selectivity and/or easier preparation, those novel compounds of the formula (I) mentioned or their salts are of particular interest in which individual radicals have one of the preferred meanings already mentioned or mentioned hereinbelow, or, in particular, those, in which one or more of the preferred meanings already mentioned or mentioned hereinbelow are combined.

Of interest are, for example, compounds of the formula (I) according to the invention or salts thereof in which

R¹ is (C₁-C₆)alkyl, preferably (C₁-C₄)alkyl, or (C₃-C₆)cycloalkyl, preferably cyclopropyl, cyclobutyl, cyclopentyl or cyclohexyl, where each of the 7 last-mentioned radicals is unsubstituted or substituted by one or more halogen atoms. R¹ is for example methyl, ethyl, n- or isopropyl, 1-fluoro-1-methylethyl, 1-fluoroethyl, 1-fluoropropyl, CF₃, CC₁₃, cyclopropyl, cyclobutyl, cyclopentyl, cyclohexyl, 1-methylcyclopropyl, 1-methylcyclobutyl, 1-methylcyclopentyl or 1-methylcyclohexyl. R¹ is in particular (C₁-C₄)alkyl or (C₁-C₄)haloalkyl.

Also of interest are, for example, compounds of the formula (I) according to the invention or salts thereof in which

R² and R³ independently of one another are hydrogen, formyl, methyl, ethyl or [(C₁-C₄)alkyl]carbonyl or [(C₁-C₄)haloalkyl]carbonyl.

Preferably, one of the radicals R² and R³ is hydrogen, methyl or ethyl, preferably hydrogen, and the other of the radicals R² and R³ is hydrogen, formyl, methyl, ethyl or [(C₁-C₄)alkyl]carbonyl or [(C₁-C₄)haloalkyl]carbonyl, preferably hydrogen, formyl, acetyl, propionyl, trifluoroacetyl and trichloroacetyl, in particular hydrogen.

Of interest are, for example, compounds of the formula (I) according to the invention or salts thereof in which

R⁴ is hydrogen, (C₁-C₆)alkyl, (C₂-C₆)alkenyl, (C₂-C₆)alkynyl or (C₃-C₆)cycloalkyl, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl.

R⁴ is preferably hydrogen, (CG-C₄)alkyl, (C₂-C₄)alkenyl, (C₂-C₄)alkynyl or (C₃-C₆)cycloalkyl, in particular H, (C₁-C₄)alkyl or (C₃-C₆)cycloalkyl, where each of the 8 last-mentioned carbon-containing radicals is unsubstituted or substituted by one or more radicals selected from the group consisting of halogen and, in the case of cyclic radicals, also (C₁-C₄)alkyl and (C₁-C₄)haloalkyl.

R⁴ is in particular hydrogen, methyl, ethyl, isopropyl, n-propyl, 1-fluoro-n-propyl, 1-fluoro-1-methylethyl, 1-fluoroethyl, trifluoromethyl, cyclopropyl, 1-fluorocyclopropyl, 2,2-difluorocyclopropyl, 2,2-dimethylcyclopropyl, cyclobutyl, especially hydrogen, methyl, ethyl, isopropyl, cyclopropyl or cyclobutyl.

Of interest are, for example, compounds of the formula (I) according to the invention or salts thereof in which

each of the radicals R⁵, R⁶, R⁷, R⁸ and R⁹ independently of the others is hydrogen, halogen, nitro, cyano, thiocyanato, (C₁-C₄)alkyl, (C₂-C₆)alkenyl, (C₂-C₆)alkynyl, (C₃-C₆)cycloalkyl, (C₅-C₆)cycloalkenyl, phenyl or heterocyclyl, where each of the 7 last-mentioned radicals is unsubstituted or substituted, preferably unsubstituted or substituted by one or more radicals selected from the group consisting-of halogen, cyano and hydroxyl and in the case of cyclic radicals, also (C₁-C₄)alkyl and (C₁-C₄)haloalkyl, and at least one of the radicals, preferably at least two radicals, in particular 2 or 3 radicals, especially 2 radicals, of the group consisting of R⁵, R⁶, R⁷, R⁸ and R⁹ are different from hydrogen and where at least one of the radicals R⁵, R⁶, R⁷, R⁸ and R⁹ is selected from the group consisting of the radicals (C₂-C₆)alkenyl, (C₁-C₆)alkynyl, (C₃-C₆)cycloalkyl and (C₅-C₆)cycloalkenyl, where each of the 4 last-mentioned radicals is unsubstituted or substituted, preferably unsubstituted or substituted by one or more radicals selected from the group consisting of halogen, cyano and hydroxyl and, in the case of cyclic radicals, also (C₁-C₄)alkyl and (C₁-C₄)haloalkyl.

Preferably, each of the radicals from the group consisting of the radicals R⁵, R⁶, R⁷, R⁸ and R⁹ which is different from hydrogen is, independently of the others, halogen, nitro, cyano, (C₁-C₄)alkyl, (C₂-C₆)alkenyl, (C₂-C₆)alkynyl or (C₃-C₆)cycloalkyl, where each of the 4 last-mentioned radicals is unsubstituted or substituted, preferably unsubstituted or substituted by one or more radicals selected from the group consisting of halogen, cyano and hydroxyl and, in the case of cyclic radicals, also (C₁-C₄)alkyl and (C₁-C₄)haloalkyl, preferably unsubstituted or substituted by halogen, where at least one of the radicals different from hydrogen is (C₂-C₆)alkenyl, (C₂-C₆)alkynyl or (C₃-C₆)cycloalkyl, where each of the 3 last-mentioned radicals is unsubstituted or substituted, preferably unsubstituted or substituted by one or more radicals selected from the group consisting of halogen, cyano and hydroxyl and, in the case of cyclic radicals, also (C₁-C₄)alkyl and (C₁-C₄)haloalkyl. Particularly preferably, each of the radicals different from hydrogen is a radical selected from the group consisting of halogen, such as fluorine, chlorine or bromine, nitro, cyano, methyl, ethyl, n-propyl, isopropyl, 1-fluoroethyl, 1-fluoropropyl, 1-fluoro-1-methylethyl, trifluoromethyl, trichloromethyl, 1-hydroxyethyl, vinyl, allyl, 1-propen-1-yl, 1,2,2-trifluorethen-1-yl, 1,2,3,3,3-pentafluoroprop-1-en-1-yl, 1,1,2,3,3-pentafluoroprop-2-en-1-yl, 1,2,3,4,5,5,5-heptafluorobut-2-en-1-yl, 1,2-propadienyl, ethynyl, 1-propynyl, 2-propyn-1-yl (propargyl), 1-butynyl, 2-butyn-1-yl, 3-butyn-1-yl, 3-fluorobut-1-yn-1-yl, 3-hydroxybut-1-yn-1-yl, 1-pentynyl, 2-pentyn-1-yl, 3-pentyn-1-yl, 4-pentyn-1-yl, 1-hexynyl, 2-hexyn-1-yl, 3-hexyn-1-yl, 4-hexyn-1-yl, 5-hexyn-1-yl, 5-fluorohex-1-yn-1-yl, 5-hydroxyhex-1-yn-1-yl, cyclopropyl, 2,2-difluorocyclopropyl, 1 ,2,2,3,3-pentafluorocyclopropyl, cyclobutyl, cyclopentyl or cyclohexyl, where at least one radical of these is selected from the group consisting of the radicals (C₂-C₆)alkenyl, (C₂-C₆)alkynyl and (C₃-C₆)cycloalkyl, preferably (C₂-C₄)alkenyl, (C₂-C₄)alkynyl and (C₃-C₄)cycloalkyl.

Of particular interest are also compounds of the formula (I) according to the invention and salts thereof in which individual radicals have one of the meanings mentioned in the examples.

Of particular interest are also compounds of the formula (I) according to the invention and salts thereof in which in the molecule in question a plurality of the above-mentioned preferred meanings of the radicals R¹ to R⁹ are combined.

The present invention also provides processes for preparing compounds of the formula (I) or salts thereof, which comprise

(a) reacting a compound of the formula (II),

R¹—Fu (II)

in which Fu is a functional group selected from the group consisting of carboxylic ester, carboxylic orthoester, carbonyl chloride, carboxamide, carboxylic anhydride and trichloromethyl, with a biguanidide of the formula (IlI) or an acid addition salt thereof

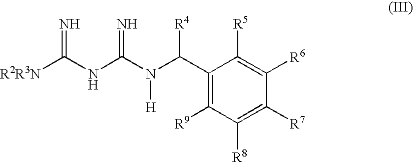

or

(b) reacting a compound of the formula (IV),

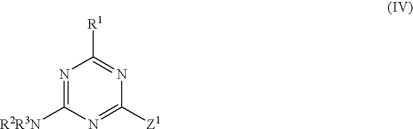

in which Z¹ is an exchangeable radical or a leaving group, for example chlorine, trichloromethyl, (C₁-C₄)alkylsulfonyl and unsubstituted or substituted phenyl-(C₁-C₄)alkylsulfonyl or (C₁-C₄)alkylphenylsulfonyl, with a suitable amine of the formula (V) or an acid addition salt thereof

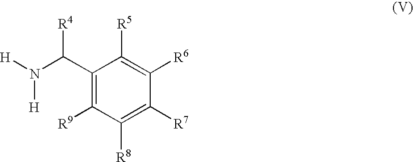

or

(c) reacting a compound of the formula (I′),

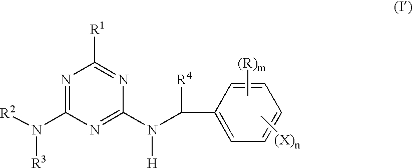

in which

X is located in the position on the phenyl ring in which in formula (I) there is a radical selected from the group of the radicals R⁵ to R⁹ representing an alkenyl, alkynyl, cycloalkyl or cycloalkenyl radical, and is a radical selected from the group consisting of halogen, such as chlorine, bromine or iodine, trifluoromethanesulfonate radical, (F₃C-SO₂—O—) boronic acid group, boronic ester group and an organometalllic radical, such as, for example, an organometallic radical containing tin

n is the number of these radicals X and

(R)ₘ are, based on the radicals (X)n the remaining radicals (m=5 minus n) from the group of the radicals R⁵ to R⁹ which, with respect to the positions and the radicals, are as defined in formula (I)

with (unsaturated or cyclic) compounds of the formula R—Y, where R has the meaning of the radical in the position X on the phenyl ring defined in formula (I) and

c1) Y=hydrogen, except when R is a cycloalkyl radical, or boronic acid group, boronic ester group or an organometallic radical, such as, for example, an organometallic radical containing tin or zinc as metal atom, in each case in the case where X is a halogen atom or a trifluoromethanesulfonate radical,

c2) Y=hydrogen, except when R is a cycloalkyl radical, or halogen or a trifluoromethanesulfonate radical, in each case in the case where X is an organometallic radical, such as, for example, an organometallic radical containing tin or zinc as metal atom, or

c3) Y=halogen or a trifluoromethanesulfonate radical, in each case in the case where X is a boronic acid group or boronic ester group

under the conditions of the Heck reaction, Suzuki reaction, Stille reaction or Sonogashira reaction or analogous conditions to give compounds of the formula (I) or salts thereof,

where in the formulae (II), (Ill), (IV), (V) and (I′) the radicals R¹, R², R³, R⁴, R⁵, R⁶, R⁷, R⁸ and R⁹ are as defined in formula (I).

According to variant (a), the compounds of the formulae (II) and (III) are preferably reacted base-catalyzed in an inert organic solvent, such as, for example, tetrahydrofuran (THF), dioxane, acetonitrile, dimethylformamide (DMF), methanol and ethanol, at temperatures between −10° C. and the boiling point of the solvent, preferably at from 20° C. to 60° C.; if acid addition salts of the formula (Ill) are used, these are generally liberated in situ with the aid of a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is employed, for example, in the range from 0.1 to 3 molar equivalents, based on the compound of the formula (III). Based on the compound of the formula (III), the compound of the formula (II) can be employed, for example, in equimolar amounts or in an excess of up to 2 molar equivalents. In principle, the corresponding processes are known from the literature (compare: Comprehensive Heterocyclic Chemistry, A. R. Katritzky, C. W. Rees, Pergamon Press, Oxford, New York, 1984, Vol.3; Part 2B; ISBN 0-08-030703-5, p.290).

According to variant (b), the compounds of the formulae (IV) and (V) are preferably reacted base-catalyzed in an inert organic solvent, such as, for example, THF, dioxane, acetonitrile, DMF, methanol and ethanol, at temperatures between −10° C. and the boiling point of the solvent or solvent mixture in question, preferably at from 20° C. to 160° C., in particular from 30° C. to 80° C., where the compound (V), if it is employed as acid addition salt, is, if appropriate, liberated in situ using a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases such as triethylamine or 1 ,8-diazabicyclo[5.4.0]undec-7-ene (DBU). The base in question is generally employed in the range from 1 to 3 molar equivalents, based on the compound of the formula (IV), and the compound of the formula (IV) can be employed, for example, in equimolar amounts or in an excess of up to 2 molar equivalents, based on the compound of the formula (V). In principle, the corresponding processes are known from the literature (cf. Comprehensive Heterocyclic Chemistry, A. R. Katritzky, C. W. Rees, Pergamon Press, Oxford, New York, 1984, Vol.3; Part 2B; ISBN 0-08-030703-5, p. 482).

The reaction of the compounds of the formula (I′) according to variant (c) is carried out, for example, using unsaturated compounds of the formula R—Y (Y=H) from the group of the alkenes or alkines or using compounds R—Y (Y=boronic acid (ester)) from the group of the alkenylboronic acids and cycloalkylboronic acids or esters thereof in the presence of organopalladium compounds under the conditions known for the C-alkylation of aromatic compounds by the Heck reaction, Suzuki reaction, Stille reaction or Sonogashira reaction or analogous conditions.

Variants result by using other catalysts known to be suitable for the type of reaction.

Alternatively, under appropriate conditions, it is also possible to use compounds R—Y in which Y is an organometallic radical, such as, for example, an organometallic radical containing tin or zinc as metal atom, in the case where X is a halogen atom or a trifuoromethanesulfonate radical.

Alternatively, under appropriate conditions, it is also possible to use compounds R—Y in which Y is halogen or a trifluoromethanesulfonate radical, where X is in each case an organometallic radical such as, for example, an organometallic radical containing tin or zinc as metal ion.

Alternatively, under appropriate conditions, it is also possible to use compounds R—Y in which Y is halogen or a trifluoromethanesulfonate radical, where X is in each case a boronic acid group or a boronic acid ester group.

Cycloalkylaromatic compounds can be generated by a large number of methods. Thus, cyclopropylaromatic compounds can be synthesized, for example, by cyclopropanation of the corresponding styrene derivatives (review on cyclopropanation: T. Aratani, Compr. Asymmetric Catal. l-1113 (1999) 1451-1460) or by coupling cyclopropylboronic acids with aryltrifluoromethanesulfonates (M. -Z. Deng, Synthesis 2000, 8, 1095-1100) or with aryl bromides (M. -Z. Deng, Angew. Chemie, 1998, 110, 20,3061-3063).

Olefins can be coupled with aromatic compounds for example by a Suzuki reaction (G. C. Fu, J. Am. Chem. Soc., 2000,122,4020-4028).

Alkynylaromatic compounds can be generated, for example, by Sonogashira coupling of an alkyn with an aryl halide or an aryltrifluoromethanesulfonate (L. Buchwald and G. C. Fu, Organic Letters, 2000, Vol. 2, No.12, 1729-1731 or N. Krause, J. Org. Chem. 1998, 63, 8551-8553).

(Kreuz) couplings with organometallic groups and/or catalysis by transition metals are described in:

Metal-catalyzed Cross-coupling Reactions, Ed.: F. Diederich; P.J. Stang.—Wiley-VCH, Weinheim 1998;

Transition Metals for Organic Synthesis, Vol.1. Ed.: M. Beller; C. Bolm, Wiley-VCH, Weinheim 1998;

Tsuji, J.: Palladium Reagents and Catalysts, John Wiley & Sons, Chichester 1995; Heck, R. F.: Palladium Reagents in Organic Synthesis.—Academic Press, New York 1985.

Further details about the Stille reaction (i.e. where one of the two coupling partners is an organotin compound) are described in:

Farina, V.; Krishnamurthy, V.; Scott, W. J., Org. React. 50 (1997),1-652;

Farina, V.; Krishnamurthy, V.; Scott, W.J.: The Stille Reaction; John Wiley & Sons, New York 1998; Stille, J. K., Angew. Chem. 98 (1986) 504-519.

The starting materials of the formulae (II), (III), (IV), (V) and (I′) are either commercially available, or they can be prepared by or analogously to processes known from the literature. The compounds can also be prepared, for example, by one of the processes described below.

The compound of the formula (IV), or a direct precursor thereof, can be prepared, for example, as follows:

1. Reaction of a compound of the formula (II) with an amidinothiourea derivative of the formula (VI),

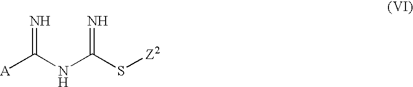

in which Z² is (C₁-C₄)alkyl or phenyl-(C₁-C₄)alkyl and A=NR²R³ as defined in formula (I) gives compounds of the formula (IV) in which Z¹=—SZ².

2. Reaction of an amidine of the formula (VII) or an acid addition salt thereof

H₂N—CR¹=NH (VII)

in which R¹ is as defined in formula (I)

with an N-cyanodithioiminocarbonate of the formula (VIII)

NC—N=C(S—Z³)₂ (VIII)

in which Z³ is (C₁-C₄)alkyl or phenyl-(C₁-C₄)alkyl gives compounds of the formula (IV) in which Z¹=—S—Z³.

3. Reaction of an alkali metal dicyanamide with a carboxylic acid derivative of the formula (II) mentioned gives compounds of the formula (IV) in which Z¹=NH₂.

4. Reaction of trichloroacetonitrile with a nitrile of the formula (IX)

R¹—CN (IX)

in which R¹ is as defined in formula (I) initially gives compounds of the formula (X)

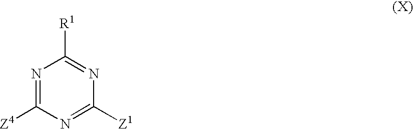

in which Z¹ and Z⁴ are each CCl₃ which, by subsequent reaction with compounds of the formula H—NR²R³ (R² and R³ are as defined in formula (I)), gives compounds of the formula (IV) in which Z′=CCl₃.

The substituted phenylalkylamines of the formula (V) required as starting materials are known and/or can be prepared by processes known per se. For example, the corresponding aromatic ketone can be converted directly into the amine by reductive amination, for example by reaction with sodium cyanoborohydride and ammonium acetate or with ammonium formate or with a mixture of ammonium formate and formic acid or with a mixture of ammonium formate, formamide and formic acid, in each case with subsequent cleavage of the amide, or the ketone is converted into an (cf. JP 11035536; JP 11043470; J. Am. Chem. Soc. 1983, 105,1578; Synthesis 1980, 695).

The reaction of the carboxylic acid derivatives of the formula (II) with the amidinothiourea derivatives of the formula (VI) is preferably carried out base-catalyzed in an organic solvent, such as, for example, acetone, THF, dioxane, acetonitrile, DMF, methanol, ethanol, at temperatures from −10° C. to the boiling point of the solvent, preferably at from 0° C. to 20° C. However, the reaction can also be carried out in water or in aqueous solvent mixtures with one or more of the abovementioned organic solvents. If (VI) is employed as acid addition salt, it can, if appropriate, be liberated in situ using a base. Suitable bases or basic catalysts are alkali metal hydroxides, alkali metal hydrides, alkali metal carbonates, alkali metal alkoxides, alkaline earth metal hydroxides, alkaline earth metal hydrides, alkaline earth metal carbonates or organic bases, such as triethylamine or 1,8-diazabicyclo[5.4.0]undec-7-ene (DBU). The base in question is, for example, employed in the range from 1 to 3 molar equivalents, based on the compound of the formula (VI). Compounds of the formula (II) and (VI) can be employed, for example, in equimolar amounts, or with an excess of up to 2 molar equivalents of the. compound of the formula (II). In principle, the corresponding processes are known from the literature (compare: H. Eilingsfeld, H. Scheuermann, Chem. Ber.; 1967, 100, 1874), the corresponding intermediates of the formula (IV) are novel.

The reaction of the amidines of the formula (VII) with the N-cyanodithioimino-carbonates of the formula (ViII) is preferably carried out base-catalyzed in an inert organic solvent, such as, for example, acetonitrile, DMF, dimethylacetamide (DMA), N-methylpyrrolidone (NMP), methanol and ethanol, at temperatures from −10° C. to the boiling point of the solvent, preferably at from 20° C. to 80° C. If (VII) is employed as acid addition salt, it can, if appropriate, be liberated in situ using a base. Suitable bases or basic catalysts are alkali metal hydroxides, alkali metal hydrides, alkali metal carbonates, alkali metal alkoxides, alkaline earth metal. hydroxides, alkaline earth metal hydrides, alkaline earth metal carbonates or organic bases, such as triethylamine or 1 ,8-diazabicyclo[5.4.0]undec-7-ene (DBU). The base in question is employed, for example, in the range from 1 to 3 molar equivalents, based on the compound of the formula (Vill), and compounds of the formulae (VII) and (VIII) can generally be employed in equimolar amounts or with an excess of two molar equivalents of the compound of the formula (II). In principle, the corresponding processes are known from the literature (compare: T. A. Riley, W. J. Henney, N. K. Dailey, B. E. Wilson, R. K. Robins; J. Heterocyclic Chem.; 1986, 23 (6), 1706-1714), the corresponding intermediates of the formula (IV) are novel.

Intermediates of the formula (X) where Z′=chlorine can be prepared by reacting alkali metal dicyanamide with a carboxylic acid derivative of the formula (II), Fu then preferably being the functional group carbonyl chloride or carboxamide. The reaction of the reaction components is carried out, for example, acid-catalyzed in an inert organic solvent, such as, for example, toluene, chlorobenzene, chlorinated hydrocarbons, at temperatures between −10° C. and the boiling point of the solvent, preferably at from 20° C. to 80° C., where the intermediates formed can be chlorinated in situ using a suitable chlorinating agent, such as, for example, phosphorus oxychloride. Suitable acids are, for example, hydrohalic acids, such as HCl, or else Lewis acids, such as, for example, AICl₃ or BF₃ (compare U.S. Pat. No. 5,095,113, DuPont).

Intermediates of the formula (X) where Z¹, Z⁴=trihalomethyl can be prepared by reacting the corresponding trihaloacetonitriles with a carbonitrile of the formula (IX). The reaction of the reaction components is carried out, for example, acid-catalyzed in an inert organic solvent, such as, for example, toluene, chlorobenzene, chlorinated hydrocarbons, at temperatures between −40° C. and the boiling point of the solvent, preferably at from −10° C. to 30° C. Suitable acids are, for examnple, hydrohalic acids, such as HCl, or else Lewis acids, such as, for example, AlCl₃ or BF₃ (cf. EP-A-130939, Ciba Geigy).

Intermediates of the formula (IV), in which Z¹=(C₁-C₄)alkylmercapto or unsubstituted phenyl-(C₁-C₄)alkylmercapto, can be converted in an inert organic solvent, such as, for example toluene, chlorobenzene, chlorinated hydrocarbons or others, at temperatures between −40° C. and the boiling point of the solvent, preferably at from 20° C. to 80° C., with a suitable chlorinating agent, such as, for example elemental chlorine or phosporus oxychloride, into more reactive chlorotriazines of the formula (IV), in which Z¹=Cl (cf. J. K. Chakrabarti, D. E. Tupper; Tetrahedron 1975, 31(16), 1879-1882).

Intermediates of the formula (IV), in which Z¹=(C₁-C₄)alkylmercapto or unsubstituted or substituted phenyl-(C₁-C₄)alkylmercapto or (C₁-C₄)alkylphenylthio can be oxidized in a-suitable solvent, such as, for example, chlorinated hydrocarbons, acetic acid, water, alcohols, acetone or mixtures thereof at temperatures between 0° C. and the boiling point of the solvent, preferably at from 20° C. to 80° C., with a suitable oxidizing agent, such as, for example, m-chloroperbenzoic acid, hydrogen peroxide, potassium peroxomonosulfate (compare: T. A. Riley, W. J. Henney, N. K. Dailey, B. E. Wilson, R. K. Robins; J. Heterocyclic Chem.; 1986, 23 (6),1706-1714).

The compounds of the formula (III) can be prepared from compounds of the formula (V) and/or acid adducts thereof by reaction with cyanoguanide (“dicyandiamide”) of the formula (XI),

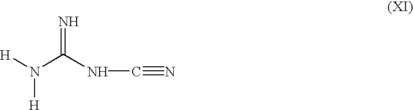

if appropriate in the presence of a reaction auxiliary, such as, for example, hydrochloride, and if appropriate in the presence of a diluent, such as, for example, n-decane or 1,2-dichlorobenzene, at temperatures of, for example, from 100° C. to 200° C. (cf. EP-A-492615, Preparation Examples).

The amines of the formula (V) or corresponding precursors for the compounds of the formula (I′) can be synthesized from simple structural building blocks as precursors following known methods. The amino group can be obtained, for example, from corresponding ketones by reductive amination (cf. the literature mentioned above, e.g. on page 1, on aminotriazine herbicides).

Some optically active aminotriazines of the formula (I) and salts thereof (hereinbelow together in short also referred to as “compounds (I) according to the invention” or “compounds (I)”) can be prepared analogously to optically active aminotriazines which are already known from the literature mentioned above; cf. specifically optically active compounds from DE-A-19810349. The international application No. PCT/EPOO/1 1861 (WO-A-01/44208) also proposes optically active compounds (I).

The compounds (I) can be prepared analogously to the methods described therein or analogously to known methods, as described in the patent publications also mentioned further above and in the literature cited therein.

With respect to the preferred compounds, their preparation and general conditions for their use and in particular with respect to the specific compounds given as examples, reference is made to the descriptions of the publications mentioned, and in this respect, these descriptions are incorporated into the present invention.

Optically active compounds (I) can be obtained, for example, from optically active biguanides by reaction of optically active amines and cyanoguanidine of the formula H₂N—C(═NH)—NH—CN (see, for example, EP-A-492615). In general, the reaction can be carried out efficiently under acid catalysis and in the presence of an organic solvent, such as an optionally halogenated hydrocarbon. Suitable catalysts are, for example, mineral acids, such as hydrogen chloride; suitable solvents are, for example, dichloromethane or n-decane. The reaction is carried out, for example in the range from 0 to 200° C., preferably from 90 to 180° C.

The optically active amines required for the above reaction and for preparation variant b) are known or can be prepared by processes known per se (cf. Tetrahedron Lett. 29 (1988) 223-224, Tetrahedron Left. 36 (1995) 3917-3920; Tetrahedron, Asymmetry 5 (1994) 817-820; EP-A-320898, EP-A-443606, DE-A-3426919, DE-A-400610).

Optically active compounds can also be obtained by customary methods for optical resolutions (cf. textbooks of stereochemistry), for example following processes for separating mixtures into diastereomers, for example physical processes, such as crystallization, chromatographic processes, in particular column chromatography and high pressure liquid chromatography, distillation, if appropriate under reduced pressure, extraction and other processes, it is possible to separate the remaining mixtures of enantiomers, generally by chromatographic separation on chiral solid phases. Suitable for preparative amounts or on an industrial scale are processes such as the crystallization of diastereomeric salts which can be obtained from the compounds (I) using optically active acids and, if appropriate, provided that acidic groups are present, using optically active bases.

Optically active acids which are suitable for optical resolution by crystallization of diastereomeric salts are, for example, camphorsulfonic acid, camphoric acid, bromocamphorsulfonic acid, quinic acid, tartaric acid, dibenzoyltartaric acid and other analogous acids; suitable optically active bases are, for example, quinine, cinchonine, quinidine, brucine, 1-phenylethylamine and other analogous bases.

The crystallizations are then in most cases carried out in aqueous or aqueous-organic solvents, where the diastereomer which is less soluble precipitates first, if appropriate after seeding. One enantiomer of the compound of the formula (I) is then liberated from the precipitated salt, or the other is liberated from the crystals, by acidification or using base.

The following acids are suitable for preparing the acid addition salts of the compounds of the formula (I): hydrohalic acids, such as hydrochloric acid or hydrobromic acid, furthermore phosphoric acid, nitric acid, sulfuric acid, mono- or bifunctional carboxylic acids and hydroxycarboxylic acids, such as acetic acid, maleic acid, succinic acid, fumaric acid, tartaric acid, citric acid, salicylic acid, sorbic acid or lactic acid, and also sulfonic acids, such as p-toluenesulfonic acid and 1,5-naphthalenedisulfonic acid. The acid addition compounds of the formula (I) can be obtained in a simple manner by the customary methods for forming salts, for example by dissolving a compound of the formula (I) in a suitable organic solvent, such as, for example, methanol, acetone, methylene chloride or benzine, and adding the acid at temperatures from 0 to 100° C., and they can be isolated in a known manner, for example by filtration, and, if appropriate, purified by washing with an inert organic solvent.

The base addition salts of the compounds of the formula (I) are preferably prepared in inert polar solvents, such as, for example, water, methanol or acetone, at temperatures from 0 to 100° C. Examples of bases which are,suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia or ethanolamine.

Solvents referred to as “inert solvents” in the above process variants are to be understood as meaning in each case solvents which are inert under the reaction conditions in question, but which need not be inert under any reaction conditions.

A collection of compounds (I) which can be synthesized by the above mentioned process may also be prepared in a parallel manner where the process may be carried out manually, in a partially automated manner or in a fully automated manner. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom, 1997, pages 69 to 77.

A number of commercially available apparatuses as they are offered by, for example, Stem Corporation, Woodrolfe Road, Tollesbury, Essex, CM9 8SE, England, or H+P Labortechnik GmbH, Bruckmannring 28, 85764 Oberschleiβheim, Germany may be used for the parallel procedure of the reaction and work-up. For the parallel purification of compounds (I), or of intermediates obtained during the preparation, use may be made, inter alia, of chromatography apparatuses, for example those from ISCO, Inc., 4700 Superior Street, Lincoln, Nebr. 68504, USA. The apparatuses mentioned lead to a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to what has been described here, compounds (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Verlag Academic Press, 1998. The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Joila, Calif. 92037, USA, are employed, may be partially automated. The automation of solid-phase-supported parallel synthesis is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation methods described here give compounds (I) in the form of collections of substances known as libraries. The present invention also relates to libraries of the compounds (I) which contain at least two compounds (I) and their intermediates.

The compounds of the formula (I) according to the invention and their salts, hereinbelow together referred to as compounds of the formula (I) (according to the invention), have excellent herbicidal activity against a broad spectrum of economically important monocotyledonous and dicotyledonous harmful plants. The active compounds also act efficiently on perennial weeds which produce shoots from rhizomes, root stocks or other perennial organs and which are difficult to control. In this context, it is immaterial whether the substances are applied pre-sowing, pre-emergence or post-emergence.

Specifically, examples may be mentioned of some representatives of the monocotyledonous and dicotyledonous weed flora which can be controlled by the compounds according to the invention, without these being a restriction to certain species.

Examples of weed species on which the active compounds act efficiently are, from amongst the monocotyledons, Avena spp., Alopecurus spp., Brachiaria spp., Digitaria spp., Lolium spp., Echinochloa spp., Panicum spp., Phalaris spp., Poa spp., Setaria spp., and also Cyperus species from the annual sector, and from amongst the perennial species Agropyron, Cynodon, Imperata and Sorghum, and also perennial Cyperus species.

In the case of the dicotyledonous weed species, the spectrum of action extends to species such as, for example, Abutilon spp., Amaranthus spp., Chenopodium spp., Chrysanthemum spp., Galium spp., lpomoea spp., Kochia spp., Lamium spp., Matricaria spp., Pharbitis spp., Polygonum spp., Sida spp., Sinapis spp., Solanum spp., Stellaria spp., Veronica spp,. Viola spp., and Xanthium spp., from amongst the annuals, and Convolvulus, Cirsium, Rumex and Artemisia in the case of the perennial weeds.

The active ingredients according to the invention also effect outstanding control of harmful plants which occur under the specific conditions of rice growing such as, for example, Sagittaria, Alisma, Eleocharis, Scirpus and Cyperus.

If the compounds according to the invention are applied to the soil su rface prior to germination, then the weed seedlings are either prevented completely from emerging, or the weeds grow until they have reached the cotyledon stage but then their growth stops, and, eventually, after three to four weeks have elapsed, they die completely.

If the active compounds are applied post-emergence to the green parts of the plants, growth also stops drastically a very short time after the treatment and the weed plants remain at the developmental stage of the point in time of application, or they die completely after a certain time, so that in this manner competition by the weeds, which is harmful to the crop plants, is eliminated at a very early point in time and in a sustained manner.

Although the compounds according to the invention have an excellent herbicidal activity against monocotyledonous and dicotyledonous weeds, crop plants of economically important crops, for example wheat, barley, rye, rice, corn, sugar beet, cotton and soybean, are not damaged at all, or only to a negligible extent. For these reasons, the present compounds are highly suitable for selectively controlling undesired plant growth in plantings of agriculturally useful plants.

In addition, the substances according to the invention have outstanding growth-regulating properties in crop plants. They engage in the plant metabolism in a regulating manner and can thus be employed for the targeted control of plant constituents and for facilitating harvesting, such as for example by provoking desiccation and stunted growth. Furthermore, they are also suitable for generally regulating and inhibiting undesirable vegetative growth, without destroying the plants in the process. Inhibition of vegetative growth plays an important role in many monocotyledonous and dicotyledonous crops because lodging can be reduced hereby, or prevented completely.

Owing to their herbicidal and plant growth-regulatory properties, the active compounds can also be employed for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms such as fungi, bacteria or viruses. Other particular properties relate, for example, to the quantity, quality, storage-stability, composition and to specific ingredients of the harvested product. Thus, transgenic plants having an increased starch content or a modified quality of the starch or those having a different fatty acid composition of the harvested product are known.

The use of the compounds of the formula (I) according to the invention or their salts in economically important transgenic crops of useful and ornamental plants, for example of cereal, such as wheat, barley, rye, oats, millet, rice, maniok and corn, or else in crops of sugar beet, cotton, soybean, oilseed rape, potato, tomato, pea and other vegetable species is preferred.

The compounds of the formula (I) can preferably be used as herbicides in crops of useful plants which are resistant or which have been made resistant by genetic engineering toward the phytotoxic effects of the herbicides.

Conventional ways of preparing novel plants which have modified properties compared to known plants comprise, for example, traditional breeding methods and the generation of mutants. Alternatively, novel plants having modified properties can be generated with the aid of genetic engineering methods (see, for example, EP-A 0 221 044, EP-A 0 131 624). For example, there have been described several cases of

genetically engineered changes in crop plants in order to modify the starch synthesized in the plants (for example WO 92/11376, WO 92/14827 and WO 91/19806),

transgenic crop plants which are resistant to certain herbicides of the glufosinate type (cf., for example, EP-A 0 242 236, EP-A 0 242 246) or glyphosate type (WO 92/00377), or of the sulfonylurea type (EP-A 0 257 993, U.S. Pat. No. 5,013,659),

transgenic crop plants, for example cotton, having the ability to produce Bacillus thuringiensis toxins (Bt toxins) which impart resistance to certain pests to the plants (EP-A 0 142 924, EP-A 0 193 259),

transgenic crop plants having a modified fattv acid composition (WO 91/13972).

Numerous molecular biological techniques which allow the preparation of novel transgenic plants having modified properties are knowri in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim, 2nd edition 1996, or Christou, “Trends in Plant Science” 1 (1996) 423-431).

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or -by expressing at least one appropriately constructed ribozyme which specifically cleaves transcripts of the abovementioned gene product.

To this end, it is possible to employ both DNA molecules which comprise the entire coding sequence of a gene product including any flanking 'sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical.

When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The compounds (I) according to the invention can preferably be used in transgenic crops which are resistant to herbicides selected from the group consisting of the sulfonylureas, imidazolin-ones, glufosinate-ammoniurn or glyphosate-isopropylammonium and analogous active compounds.

When using the active compounds according to the invention in transgenic crops, in addition to the effects against harmful plants which can be observed in other crops, there are frequently effects which are specific for the application in the respective transgenic crop, for example a modified or specifically broadened spectrum of weeds which can be controlled, modified application rates which can be used for the application, preferably good combinability with the herbicides to which the transgenic crop is resistant, and an effect on the growth and the yield of the transgenic crop plants.

The invention therefore also provides for the use of the compounds (I) according to the invention as herbicides for controlling harmful plants in transgenic crop plants.

The compounds according to the invention can be applied in the customary formulations in the form of wettable powders, emulsifiable concentrates, sprayable solutions, dusts or granules. The invention therefore also provides herbicidal and plant growth-regulating compositions comprising compounds of the formula (I).

The compounds of the formula (I) can be formulated in various ways depending on the prevailing biological and/or chemicophysical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules and waxes.

These individual formulation types are known in principle and are described, for example, in Winnacker-Kuchler, “Chemische Technologie” [Chemical Technology], Volume 7, C. Hauser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H.v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J., Sislev and Wood, “Encyclopedia of Surface Active Agents”, Chem. Publ. Co. Inc., N.Y. 1964; Schönfeldt, “Grenzflächenaktive Athylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th edition 1986.

Based on these formulations it is also possible to produce combinations with other pesticidally active substances, for example insecticides, acaricides, herbicides and fungicides, and also with safeners, fertilizers and/or growth regulators, for example in the form of a ready-mix or tank mix.

Wettable powders are preparations which are uniformly dispersible in water and which, in addition to the active compound and as well as a diluent or inert substance, also contain surfactants of ionic and/or nonionic type (wetting agents, dispersants), for example polyethoxylated alkyl phenols, polyethoxylated fatty alcohols, polyethoxylated fatty amines, fatty alcohol polyglycol ethersulfates, alkanesulfonates, alkylbenzenesulfonates, sodium ligninsulfonate, sodium 2,2′-dinaphthylmethane-6,6′-disulfonate, sodium dibutyinaphthalenesulfonate or else sodium oleoylmethyltaurinate. To prepare the wettable powders, the herbicidally active compounds are finely ground, for example in customary apparatuses- such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents, with the addition of one or more surfactants of ionic and/or nonionic type (emulsifiers). Examples of emulsifiers which can be used are calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters.

Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material. For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, in particular from 0.1 to 95% by weight, of active compound of the formula (I).

In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

The compounds of the formula (I) or their salts can be used as such or combined in the form of their preparations (formulations) with other pesticidally active compounds, such as, for example, insecticides, acaricides, nematicides, herbicides, fungicides, safeners, fertilizers and/or growth regulators, for example as finished formulations or tank mixes.

Suitable active compounds which can be combined with the active compounds according to the invention in mixed formulations or in a tank mix are, for example, known active compounds, whose effect is based on an inhibition of, for example, acetolactate synthase, acetyl-coenzyl-A carboxylase, PS I, PS II, HPPDO, phytoene desaturase, protoporphyrinogen oxidase, glutamine synthetase, cellulose biosynthesis, 5-enolpyruvylshikimate-3-phosphate synthetase. Such compounds, and also other compounds that can be used, with a mechanism of action that is, in some cases, unknown or different, are described, for example, in Weed Research 26, 441-445 (1986), or in “The Pesticide Manual”, 12h edition 2000, published by

The British Crop Protection Council (hereafter also abbreviated to “PM”), and in the literature cited therein. For example, the following active compounds may be mentioned as herbicides which are known from the literature.and which can be combined with the compounds of the formula (I) (note: the compounds are either referred to by the common name in accordance with the International Organization for Standardization (ISO) or by the chemical names, if appropriate together with a customary code number):

acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; alachlor; alloxydim(-sodium); ametryn; amicarbazone; amidochlor, amidosulfuron; amitrol; AMS, i.e. ammonium sulfamate; anilofos; asulam; atrazine; azafenidin; azimsulfuron (DPX-A8947); aziprotryn; barban; BAS 516 H, i.e. 5-fluoro-2-phenyl-4H-3, 1-benzoxazin4-one; beflubutamide; benazolin(-ethyl); benfluralin; benfuresate; bensulfuron(-methyl); bensulide; bentazone; benzobicyclone; benzofenap; benzofluor; benzoylprop(-ethyl); benzthiazuron; bialaphos; bifenox; bispyribac(-sodium); bromacil; bromobutide; bromofenoxim; bromoxynil; bromuron; buminafos; busoxinone; butachlor; butafenacil; butamifos; butenachlor; buthidazole; butralin; butroxydim; butylate; cafenstrole (CH-900); carbetamide; carfentrazone(-ethyl)(ICl A0051); caloxydim; CDM, i.e. 2-chloro-N,N-di-2-propenylacetamide; CDEC, i.e. 2-chloroallyl diethyldithiocarbamate; chlomethoxyfen; chloramben; chlorazifop-butyl; chlormesulon(ICl A0051 ); chlorbromuron; chlorbufam; chlorfenac; chlorflurecol-methyl; chloridazon; chlorimuron(-ethyl); chlornitrofen; chlorotoluron; chloroxuron; chlorpropham; chlorsulfuron; chlorthal-dimethyl; chlorthiamid; chlortoluron, cinidon(-ethyl and -methyl); cinmethylin; cinosulfuron; clefoxydim; clethodim; clodinafop and its ester derivatives (for example clodinafop-propargyl); clomazone; clomeprop; cloproxydim; clopyralid; clopyrasulfuron(-methyl); cloransulam(-methyl); cumyluron (JC 940); cyanazine; cycloate; cyclosulfamuron (AC 104); cycloxydim; cycluron; cyhalofop and its ester derivatives (for example cyhalofop-butyl, DEH-1 12); cyperquat; cyprazine; cyprazole; daimuron; 2,4-D; 2,4-DB; 2,4-DB, dalapon; desmedipham; desmetryn; di-allate; dicamba; dichlobenil; dichlorprop; diclofop- and its esters such as diclofop-methyl; diclosulam, diethatyl(-ethyl); difenoxuron; difenzoquat; diflufenican; diflufenzopyr; dimefuron; dimepiperate; dimethachlor; dimethametryn; dimethenamid (SAN-582H); dimethazone, dimexyflam, dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-d imethylethyl)-N-methyl-1 H-pyrazole-4-carboxamide; endothal; epoprodan; EPTC; esprocarb; ethalfluralin; ethametsulfuron(-methyl); ethidimuron; ethiozin; ethofumesate; ethoxyfen and its esters (for example the ethyl ester, HN-252); ethoxysulfuron; etobenzanid (HW 52); F5231, i.e. N-[2-chloro-4-fluoro-5-[4-(3-fluoropropyl)-4,5-dihydro-5-oxo-1 H-tetrazol-1-yl]phenyl]ethanesulfonamide; fenoprop; fenoxan, fenoxaprop and fenoxaprop-P and their esters, for example fenoxaprop-P-ethyl and fenoxaprop-ethyl; fenoxydim; fentrazamide; fenuron; flamprop(-methyl or -isopropyl or -isopropyl-L); flazasulfuron, floazulate, florasulam; fluazifop and fluazifop-P and their esters for example fluazifop-butyl and fluazifop-P-butyl; flucarbazone(-sodium); fluchloralin; flumetsulam; flumeturon; flumiclorac(-pentyl); flumioxazin (S-482); flumipropyn; fluometuron; fluorochloridone, fluorodifen; fluoroglycofen(-ethyl); flupoxam (KNW-739); flupropacil (UBIC-4243), flupyrsulfuron(-methyl, or -sodium); flurenol(-butyl); fluridone; flurochloridone; fluroxypyr(-meptyl); flurprimidol, flurtamone; fluthiacet(-methyl); fluthiamide; fomesafen; foramsulfuron; fosamine; furyloxyfen; glufosinate(-ammonium); glyphosate(-isopropylammonium); halosafen; halosulfuron(-methyl) and its esters (for example the methyl ester, NC-319); haloxyfop and its esters; haloxyfop-P (=R-haloxyfop) and its esters; hexazinone; imazamethabenz(-methyl); imazapyr; imazaquin and salts such as the ammonium salt; imazamethapyr; imrazamox; imazapic; imazethamethapyr; imazethapyr; imazosulfuron; indanofan; ioxvnil; isocarbamid; isoiprop)alin: isoproturon; isouron; isoxaben; isoxachlortole; isoxaflutole; isboxapyrifop; karbutilate; lactofen; lenacil; linuron; MCPA; MCPB; mecoprop; mefenacet; mefliidid; mesosulfuron, mesotrione; metamitron; metazachlor; methabenzthiazuron; metham; methazole; methoxyphenone; methyldymron; metabenzuron, methobenzuron; metobromuron; (alpha-)metolachlor; metosulam (XRD 511); meetoxuron; metribuzin; metsulfuron-methyl; MH; molinate; monalide; monocarbamide dihydrogensulfate; monolinuron; monuron; MT 128, i.e. 6-chloro-N-(3-chloro-2-propenylj-5-methyl-N-phenyl-3-pyridazinamine; MT 5950, i.e. N-[3-chloro4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazodn; orbencarb; oryzalin; oxadiargyl (RP-020630); oxadiazone; oxasulfuron; oxaciclomefone; oxyfluorfen; paraquat; pebulate; pelargonic acid; pendimethalin; pentoxazone; perFluidone; phenisopham; phenmedipham; picloram; picolinafen; piperophos; piributicarb; pirifenop-butyl; pretilachlor; primisulfuron(-methyl); procarbazone(-sodium); procyazine; prodiamine; profluralin; proglinazine(-ethyl); prometon; prometryn; propachlor; propanil; propaquizafop and its esters; propazine; propham; propisochlor; propyzamide; prosulfalin; prosulfocarb; prosulfuron (CGA-152005); prynachlor; pyroflufen(-ethyl); pyrazolinate; pyrazon; pyrazosulfuron(-ethyl); pyrazoxyfen; pyribenzoxim; pyributicarb; pyridafol; pyridate; pyriminobac(-methyl); pyrithiobac(-sodium) (KIH-2031); pyroxofop and its esters (for example propargyl ester); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro4-(trifluoromethyl)phenoxy]-2-naphthalenyl]oxy]propanoic acid and its methyl ester; sulcotrione; sulfentrazone (FMC-97285, F-6285); sulfazurone; sulfometuron(-methyl); sulfosate (ICl-A0224); sulfosulfuron; TCA; tebutam (GCP-5544); tebuthiuron; tepraloxydim; terbacil; terbucarb; terbuchior; terbumeton; terbuthylazine; terbutryn; TFH 450, i.e. N,N-diethyl-3-[(2-ethyl-6-methylphenyl)sulfonyl]-1 H-1,2.4-triazole-1-carboxamide; thenylchlor (NSK-850); thiafluamide; thiazafluron; thiazopyr (Mon-13200); thidiazimin (SN-24085); thifensulfuron(-methyl); thiobencarb; tiocarbazil; tralkoxydim; tin-allate; triasulfuron; triaziflam; triazofenamide; tribenuron(-methyl); triclopyr; tridiphane; trietazine; trifluralin; triflusulfuron and esters (e.g. the methyl ester, DPX-66037); trimeturon; tritosulfuron; tsitodef; vernolate; WL 110547, i.e. 5-phenoxy-1-[3-(trifluoromethyl)phenyl]-1 H-tetrazole; BAY MKH 6561, UBH-509; D-489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127 and KIH-2023.

Of particular interest is the selective control of harmful plants in crops of useful and ornamental plants. Although the compounds (I) according to the invention have very good to satisfactory selectivity in a large number of crops, it is possible in principle that phytoxicity in the crop plants can occur in some crops and, in particular, when the compounds (I) are mixed with other herbicides which are less selective. In this respect, the combinations of the compounds (I) according to the invention which contain the compounds (I), or their combinations with other herbicides or pesticides, and safeners are of particular interest. The safeners, which are employed in such amounts that they act as antidotes, reduce the phytotoxic side effects of the herbicides/pesticides used, for example in economically important crops such as cereals (wheat, barley, rye, corn, rice, millet), sugar beet, sugar cane, oilseed rape, cotton and soybean, preferably cereal. Suitable safeners for the compounds (I) and their combinations with other pesticides are, for example, the following groups of compounds:

a) Compounds of the dichlorophenylpyrazoline-3-carboxylic acid !type, preferably compounds such as ethyl 1-(2,4-dichlorophenyl)-5-(ethoxycarbonyl)-5-methyl-2-pyrazoline-3-carboxylate (S1-1) (“mefenpyr-diethyl”, PM, pp. 781-782), and related compounds, as described in WO 91/07874,

b) Derivatives of dichlorophenylpyrazole carboxylic acid, preferably compounds such as ethyl 1-(2,4-dichlorophenyl)-5-methylpyrazole-3-carboxylate (S1-2), ethyl 1-(2.4-dichlorophenyl)-5-isopropylrpvr2zole-3-carhoxyvlat (f1-3), ethyl 1-(2,4-dichlorophenyl)-5-(1 ,1-dimethylethyl)pyrazole-3-carboxylate (S 1-4), ethyl 1-(2,4-dichlorophenyl)-5-phenylpyrazole-3-carboxylate (S1-5) and related compounds as described in EP-A-333 131 and EP-A-269 806.

c) Compounds of the triazolecarboxylic acid type, preferably compounds such as fenchlorazole(ethyl ester), i.e. ethyl 1-(2,4-dichlorophenyl)-5-trichloromethyl-(1 H)-1,2,4-triazole-3-carboxylate (S1-6) and related compounds as described in EP-A-174 562 and EP-A-346 620.

d) Compounds of the 5-benzyl- or ⁵-phenyl-2-isoxazoline-3-carboxylic acid type, or the 5,5-diphenyl-2-isoxazoline-3-carboxylic acid type, preferably compounds such as ethyl 5-(2,4-dichlorobenzyl)-2-isoxazoline-3-carboxylate (S1-7) or ethyl 5-phenyl-2-isoxazoline-3-carboxylate (SI1-8) and related compounds, as described in WO 91/08202, or ethyl 5,5-diphenyl-2-isoxazolinecarboxylate (S1-9) (“isoxadifen-ethyl”) or its -n-propyl ester (S1-10) or ethyl 5-(4-fluorophenyl)-5-phenyl-2-isoxazoline-3-carboxylate (S1-11), as described in the German patent application (WO-A-95/07897).

e) Compounds of the 8-quinolineoxyacetic acid type (S2), preferably 1-methylhex-1-yl (5-chloro-8-quinolineoxy)acetate (common name “cloquintocet-mexyl”) (S2-1) (see PM, pp. 263-264) 1,3-dimethylbut-1-yl (5-chloro-8-quinolineoxy)acetate (S2-2), 4-allyloxybutyl (5-chloro-8-quinolineoxy)acetate (S2-3), 1-allyloxyprop-2-yl (5-chloro-8-quinolineoxy)acetate (S2-4), ethyl (5-chloro-8-quinolineoxy)acetate (S2-5), methyl (5-chloro-8-quinolineoxy)acetate (S2-6), allyl (5-chloro-8-quinolineoxy)acetate (S2-7), 2-(2-propylideneiminoxy)-1-ethyl (5-chloro-8-quinolineoxy)acetate (S2-8), 2-oxoprop-1-yl (5-chloro-8-quinolineoxy)acetate (S2-9) and related compounds, as described in EP-A-86 750, EP-A-94 349 and EP-A-191 736 or EP-A-0 492 366.

f) Compounds of the (5-chloro-8-quinolineoxy)malonic acid type, preferably compounds such as diethyl (5-chloro-8-quinolineoxy)rnalonate, diallyl (5-chloro-8-quinolineoxy)malonate, methyl ethyl (5-chloro-8-quinolineoxy)malonate and related compounds, as described in EP-A-0 582 198.

g) Active comounds of the phenoxyacetic or -propionic acid derivative type or the aromatic carboxylic acid type, such as, for example, 2,4-dichlorophenoxyacetic acid (esters) (2,4-D), 4-chloro-2-methylphenoxypropionic esters (Mecoprop), MCPA or 3,6-dichloro-2-methoxybenzoic acid (esters) (Dicamba).

h) Active compounds of the pyrimidine type, which are used as soil-acting safeners in rice, such as, for example, “fenclorim” (PM, pp. 512-511) (=4,6-dichloro-2-phenylpyrimidine), which is known as safener for pretilachlor in sown rice,

i) Active compounds of the dichloroacetamide type, which are frequently used as pre-emergent safeners (soil-acting safeners), such as, for example, “dichlormid” (PM, pp. 363-364) (=N,N-diallyl-2,2-dichloroacetamide), “R-29148” (=3-dichloroacetyl-2,2,5-trimethyl-1,3-oxazolidine from Stauffer), “benoxacor” (PM, pp. 102-103) (=4-dichloroacetyl-3,4-dihydro-3-methyl-2H-1,4-benzoxazine), “PPG-1292” (=N-allyl-N-[(1,3-dioxolan-2-yl)methyl]dichloroacetamide from PPG Industries), “DK-24” (=N-allyl-N-[(allylaminocarbonyl)methyl]dichloroacetamide from Sagro-Chem), “AD-67” or “MON 4660” (=3-dichloroacetyl-1-oxa-3-aza-spiro[4,5]decane from Nitrokemia or Monsanto), “diclonon” or “BAS145138” or “LAB145138” (=3-dichloroacetyl-2,5,5-trimethyl-1,3-diazabicyclo[4.3.0]nonane from BASF) and “furilazol” or “MON 13900” (see PM, 637-638) (=(RS)-3-dichloroacetyl-5-(2-furyl)-2,2-dimethyloxazolidine)

j) Active compounds of the type of the dichloroacetone derivatives, such as, for example, “MG 191” (CAS-Reg. No. 96420-72-3) (=2-dichloromethyl-2-methyl-1,3-dioxolane from Nitrokemia), which is known as safener for corn,

k) Active compounds of the oxyimino compound type, which are known as seed dressings, such as, for example, “oxabetrinil” (PM, pp. 902-903) (=(Z)-1,3-dioxolan-2-ylmethoxy-imino(phenyl)acetonitrile), which is known as seed dressing safener for millet against metolachlor damage, “fluxofenim” (PM, pp. 613-614) (=1-(4-chlorophenyl)-2,2,2-trifluoro-1-ethanone O-(1,3-dioxolan-2-ylmethyl) oxime), which is known as seed dressing safener for millet against metolachlor damage, and “cyometrinil” or “-CGA-43089” (PM, p. 1304) (=(Z)-cyanomethoxyimino(phenyl)acetonitrile), which is known as seed dressing safener for millet against metolachlor damage,

l) Active compounds of the thiazolecarboxylic ester type, which are known as seed dressings, such as, for example, “flurazol” (PM, pp. 590-591) (=benzyl 2-chloro4-trifluoromethyl-1,3-thiazole-5-carboxylate), which is known as seed dressing safener for millet against alachlor and metolachlor damage,

m) Active compounds of the naphthalenedicarboxylic acid derivative type, which are known as seed dressings, such as, for example, “naphthalic anhydride” (PM, p. 1342) (=1,8-naphthalenedicarboxylic anhydride), which is known as seed dressing safener for corn against thiocarbamate herbicide damage,

n) Active compounds of the chromanacetic acid derivative type, such as, for example, “CL 304415” (CAS-Reg. No. 31541-57-8) (=2-(4-carboxychroman-4-yl)acetic acid from American Cyanamid), which is known as safener for corn against imidazolinone damage,

o) Active compounds which, in addition to a herbidical action against harmful plants, also have safener action in crop plants such as rice, such as, for example, “dimepiperate” or “MY-93” (PM, pp. 404-405) (=S-1-methyl-1-phenylethyl piperidine-1-thiocarboxylate), which is known as safener for rice against damage caused by the herbicide molinate, “daimuron” or “SK 23” (PM, p. 330) (=1-(1-methyl-1-phenylethyl)-3-p-tolylurea), which is known as safener for rice against damage caused by the herbicide imazosulfuron, “cumyluron”=“JC-940” (=3-(2-chlorophenylmethyl)-1-(1-methyl-1-phenylethyl)urea, see JP-A-60087254), which is known as safener for rice against damage by some herbicides, “methoxyphenon” or “NK 049” (=3,3′-dimethyl4-methoxy-benzophenone), which is known as safener for rice against damage by some herbicides, “CSB” (=1-bromo-4-(chloromethylsulfonyl)benzene) (CAS-Reg. No. 54091-064 from Kumiai), which is known as safener against damage by some herbicides in rice

p) N-Acylsulfonamides of the formula (S3) and salts thereof,

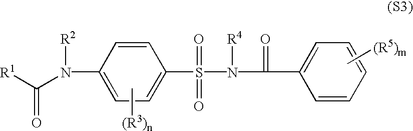

as described in WO-A-97/45016,

q) Acylsulfamoylbenzoamides of the formula (S4), if appropriate also in salt form,

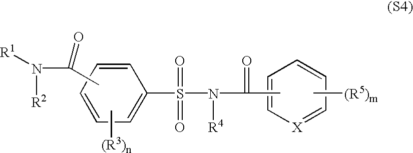

as described in the International Application No. PCT/EP98/06097, and

r) compounds of the formula (S5),

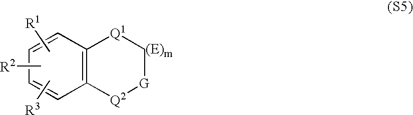

as described in WO-A 98/13 361

including the stereoisomers and the salts used in agriculture.

Among the safeners mentioned, (S1-1) and (S1-9) and (S2-1), in particular (S1-1) and (S1-9) are of particular interest.

Some of the safeners are already known as herbicides and consequently show, in addition to the herbicidal action against harmful plants, also protective action in connection with crop plants.

The ratios by weight of herbicide (mixture) to safener generally depend on the application rate of the herbicide and the efficacy of the safener in question and can vary within wide limits, for example in the range from 200:1 to 1:200, preferably 100:1 to 1:100, in particular 20:1 to 1:20. Analogously to the compounds (I) or their mixtures, the safeners can be formulated with other herbicides/pesticides and be provided and used as ready mix or tank mix with the herbicides.

For use, the formulations which are present in commercially available form are, if appropriate, diluted in the customary manner, for example using water in the case of wettable powders, emulsifiable concentrates, dispersions and water-dispersible granules. Preparations in the form of dusts, granules for soil application or broadcasting and sprayable solutions are usually not further diluted with other inert substances prior to use.

The application rate of the compounds of the formula (I) required varies with the external conditions, such as temperature, humidity, the nature of the herbicide used and the like. It can vary within wide limits, for example between 0.001 and 10.0 kg/ha or more of active substance, but it is preferably between 0.005 and 5 kg/ha.

In the examples below, the amounts (including percentages) are based on weight, unless specifically defined otherwise.

A. Chemical Examples

2-Amino-4-(1-fluoro-1-methylethyl)-6-{1-[3-(3-hydroxybut-1-yn-1-yl)-4-fluorophenyl]-ethyl}-1,3,5-triazine of the formula

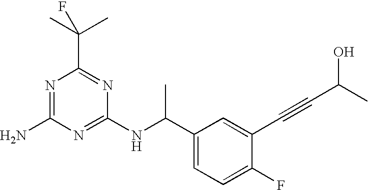

(=compound P1 7.840 from table 1)

Step 1: 4-Fluoro-3-(3-hydroxybut-1-yn-1-yl)acetophenone

10.00 9 (46.1 mmol) of 3-bromo-4-fluoroacetophenone, 4.84 g (69.1 mmol) of 1-butyn-3-ol, 1.62 g (2.3 mmol) of bis(triphenylphosphine)palladium(ll) chloride, 0.30 g (1.2 mmol) of triphenylphosphine and 6.99 g (69.1 mmol) of triethylamine in 200 ml of tetrahydrofuran was stirred for 20 min. 0.11 g (0.6 mmol) of copper(l) iodide was then added. The reaction mixture was stirred at room temperature for 10 days. The solvent was distilled off using a rotary evaporator, the residue was taken up in ethyl acetate and the organic phase was extracted with water. The aqueous phase was then extracted twice with ethyl acetate, and the organic phase was then dried and freed from the solvent using a rotary evaporator. Chromatographic purification gave 8.10 g (85% yield) of the desired product as the brown oil.

Step 2: 1-(4-Fluoro-3-(3-hydroxybut-1-yn-1-yl)phenyl)ethylamine

4.00 g (19.4 mmol) of 4-fluoro-3-(3-hydroxybut-1-yn-1-yl)acetophenone were dissolved in 80 ml of methanol. 14.95 g (194 mmol) of ammonium acetate and 2.05 g (31.0 mmol) of sodium cyanoborohydride were added, and the reaction mixture was then stirred at room temperature for 11 days. 30 ml of 1N KOH and, to complex the cyanide, a sufficient amount of iron sulfate were added. The content of the flask was stirred at room temperature for another 30 min. The mixture was filtered, the residue was washed with ethyl acetate, water was added to the filtrate and the aqueous phase was extracted three times with ethyl acetate. The organic phase was extracted three times with 1 N hydrochloric acid and the combined extracts were neutralized and extracted three times with ethyl acetate. The organic phase was dried and the filtrate was freed from the solvent using a rotary evaporator, giving 1.6 g (40% yield) of the desired product as a brown oil.

Step 3: 2-Amino-4-[1-(4-fluoro-3-(3-hydroxybut-1-yn-1-yl)phenyl)ethyl]amino-6-(2-fluoroprop-2-yl)-1 ,3,5-triazine

0.30 g (1.4 mmol) of 1-(4-fluoro-3-(3-hydroxybut-1-yn-1-yl)phenyl)ethylamine, 0.28 g (1.5 mmol) of 2-amino-4-chloro-6-(2-fluoroprop-2-yl)-1,3,5-triazine and 0.24 g (1.7 mmol) of potassium carbonate were initially charged in 20 ml of acetonitrile and heated at 79° C. for three days. The reaction mixture was filtered off with suction, the residue was washed with ethyl acetate and the filtrate was freed from the solvent using a rotary evaporator. The residue was purified-chromatographically, giving 0.19 g (35% yield) of the desired product as a white solid of melting point 119-122° C. The compounds of the formula (I) listed in the table below are prepared by or analogously to the process described above or the processes mentioned in the description.

For Table 1, concerning compounds of the formula (Ia):

(Table 1: see after pretable 1.2)

Tr—Ar (Ia)

Here, Tr and Ar are the following radicals of the formulae Tr and Ar: respectively:

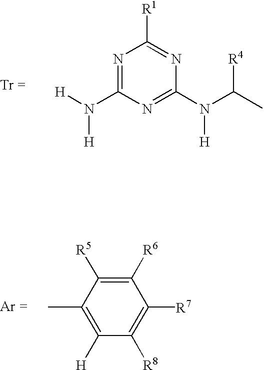

Table 1 defines individual compounds of the formula (Ia) which contain certain radicals Tr and Ar referred to by numbers. The radicals Tr-1, Tr-2, Tr-3, Tr4, etc., up to Tr-30 are indicated by the numbers 1, 2, 3, 4, etc., up to 30 appearing in the product number Px.y in place of x. The radicals No. 1, 2, 3, 4, etc., up to 1620 for Ar are indicated by the identical number in the product number Px.y in place of y.

The specific structure of the numbered radicals Tr is shown in Table 1.1 below. The structure of a radical Tr-x (for example Tr-8) is a radical of the formula Tr having the specific meanings of R¹ and R⁴, listed in Table 1.1 for R¹ (see left column) in the row in which Tr-x is located or in the Table 1.1 for R⁴ (see top row) in the column in which Tr-x is located.

TABLE 1.1
(pretable for Table 1): Definition of numbered radicals of Tr

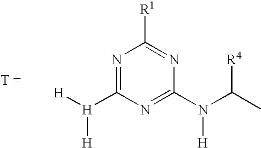

R⁴

| R¹ | H | Me | F-Et | c-Pr | c-Bu |
|---|---|---|---|---|---|
| Me | Tr-1 | Tr-2 | Tr-3 | Tr-4 | Tr-5 |
| Et | Tr-6 | Tr-7 | Tr-8 | Tr-9 | Tr-10 |
| i-Pr | Tr-11 | Tr-12 | Tr-13 | Tr-14 | Tr-15 |
| F-i-Pr | Tr-16 | Tr-17 | Tr-18 | Tr-19 | Tr-20 |
| F-Et | Tr-21 | Tr-22 | Tr-23 | Tr-24 | Tr-25 |
| F-Pr | Tr-26 | Tr-27 | Tr-28 | Tr-29 | Tr-30 |
| OMe | Tr-31 | Tr-32 | Tr-33 | Tr-34 | Tr-35 |
| OEt | Tr-36 | Tr-37 | Tr-38 | Tr-39 | Tr-40 |
| SMe | Tr-41 | Tr-42 | Tr-43 | Tr-44 | Tr-45 |
| SEt | Tr-46 | Tr-47 | Tr-48 | Tr-49 | Tr-50 |

Abbreviations for Table 1.1:

Me=methyl; Et=ethyl; i-Pr=isopropyl F-Et=1-fluoroethyl;

F-Pr=1-fluoro-n-propyl; F-i-Pr=(1-fluoro-1-methyl)ethyl;

c-Pr=cyclopropyl; c-Bu=cyclobutyl, OMe=methoxy; OEt=ethoxy;

SMe=methylthio; SEt=ethylthio

In a given column, the meaning of the radical R⁴ is identical; in a given row, the meaning of the radical R¹ is identical.

For Table 1.2 (pretable for Table 1): Definition of the radical Ar

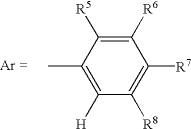

Abbreviations for Table 1.2 below:

Me=methyl; Et=ethyl; Bu=n-butyl; CHF-Me=1-fluoroethyl;

CH(OH)—Me=1-hydroxyethyl; c-Pr=cyclopropyl;

CH(CF₂-CH₂)=2,2-difluorocyclopropyl;

CF(CF₂-CF₂)=1,2,2,3,3-pentafluorocyclopropyl;

c-Bu=cyclobutyl; c-Pen=cyclopentyl; c-Hex=cyclohexyl;

CC=—CC≡C—=(ethin-1,2-diyl)

TABLE 1.2
The radicals Ar

| Ar No. | R⁵ | R⁶ | R⁷ | R⁸ |
|---|---|---|---|---|
| 1 | H | CF═CF₂ | Me | H |
| 2 | H | CF═CF₂ | Et | H |
| 3 | H | CF═CF₂ | CHF—Me | H |
| 4 | H | CF═CF₂ | i-Pr | H |
| 5 | H | CF═CF₂ | F | H |
| 6 | H | CF═CF₂ | Cl | H |
| 7 | H | CF═CF₂ | Br | H |
| 8 | H | CF═CF₂ | I | H |
| 9 | H | CF═CF₂ | NO₂ | H |
| 10 | H | CF═CF₂ | CN | H |
| 11 | H | CF═CF₂ | H | Me |
| 12 | H | CF═CF₂ | H | Et |
| 13 | H | CF═CF₂ | H | CHF—Me |
| 14 | H | CF═CF₂ | H | i-Pr |
| 15 | H | CF═CF₂ | H | F |
| 16 | H | CF═CF₂ | H | Cl |
| 17 | H | CF═CF₂ | H | Br |
| 18 | H | CF═CF₂ | H | I |
| 19 | H | CF═CF₂ | H | NO₂ |
| 20 | H | CF═CF₂ | H | CN |
| 21 | Me | H | H | CF═CF₂ |
| 22 | Et | H | H | CF═CF₂ |
| 23 | CHF—Me | H | H | CF═CF₂ |
| 24 | i-Pr | H | H | CF═CF₂ |
| 25 | F | H | H | CF═CF₂ |
| 26 | Cl | H | H | CF═CF₂ |
| 27 | Br | H | H | CF═CF₂ |
| 28 | I | H | H | CF═CF₂ |
| 29 | NO₂ | H | H | CF═CF₂ |
| 30 | CN | H | H | CF═CF₂ |
| 31 | H | Me | CF═CF₂ | H |
| 32 | H | Et | CF═CF₂ | H |
| 33 | H | CHF—Me | CF═CF₂ | H |
| 34 | H | i-Pr | CF═CF₂ | H |
| 35 | H | F | CF═CF₂ | H |
| 36 | H | Cl | CF═CF₂ | H |
| 37 | H | Br | CF═CF₂ | H |
| 38 | H | I | CF═CF₂ | H |
| 39 | H | NO₂ | CF═CF₂ | H |
| 40 | H | CN | CF═CF₂ | H |
| 41 | H | Me | CF═CF₂ | F |
| 42 | H | Me | CF═CF₂ | Cl |
| 43 | H | Me | CF═CF₂ | Br |
| 44 | H | Me | CF═CF₂ | I |
| 45 | H | Me | CF═CF₂ | Me |
| 46 | H | Et | CF═CF₂ | Et |
| 47 | H | CHF—Me | CF═CF₂ | CHF—Me |
| 48 | H | i-Pr | CF═CF₂ | i-Pr |
| 49 | H | F | CF═CF₂ | F |
| 50 | H | Cl | CF═CF₂ | Cl |
| 51 | H | Br | CF═CF₂ | Br |
| 52 | H | I | CF═CF₂ | I |
| 53 | H | NO₂ | CF═CF₂ | NO₂ |
| 54 | H | CN | CF═CF₂ | CN |
| 55 | H | CF═CF₂ | CF═CF₂ | H |
| 56 | H | CF═CF₂ | CF═CF₂ | Me |
| 57 | H | CF═CF₂ | CF═CF₂ | Et |
| 58 | H | CF═CF₂ | CF═CF₂ | CHF—Me |
| 59 | H | CF═CF₂ | CF═CF₂ | i-Pr |
| 60 | H | CF═CF₂ | CF═CF₂ | F |
| 61 | H | CF═CF₂ | CF═CF₂ | Cl |
| 62 | H | CF═CF₂ | CF═CF₂ | Br |
| 63 | H | CF═CF₂ | CF═CF₂ | I |
| 64 | H | CF═CF₂ | CF═CF₂ | NO₂ |
| 65 | H | CF═CF₂ | CF═CF₂ | CN |
| 66 | H | CF═CF₂ | H | CF═CF₂ |
| 67 | H | CF═CF₂ | Me | CF═CF₂ |
| 68 | H | CF═CF₂ | Et | CF═CF₂ |
| 69 | H | CF═CF₂ | CHF—Me | CF═CF₂ |
| 70 | H | CF═CF₂ | i-Pr | CF═CF₂ |
| 71 | H | CF═CF₂ | F | CF═CF₂ |
| 72 | H | CF═CF₂ | Cl | CF═CF₂ |
| 73 | H | CF═CF₂ | Br | CF═CF₂ |
| 74 | H | CF═CF₂ | I | CF═CF₂ |
| 75 | H | CF═CF₂ | NO₂ | CF═CF₂ |
| 76 | H | CF═CF₂ | CN | CF═CF₂ |
| 77 | H | CF═CF—CF₃ | Me | H |
| 78 | H | CF═CF—CF₃ | Et | H |
| 79 | H | CF═CF—CF₃ | CHF—Me | H |
| 80 | H | CF═CF—CF₃ | i-Pr | H |
| 81 | H | CF═CF—CF₃ | F | H |
| 82 | H | CF═CF—CF₃ | Cl | H |
| 83 | H | CF═CF—CF₃ | Br | H |
| 84 | H | CF═CF—CF₃ | I | H |
| 85 | H | CF═CF—CF₃ | NO₂ | H |
| 86 | H | CF═CF—CF₃ | CN | H |
| 87 | H | CF═CF—CF₃ | H | Me |
| 88 | H | CF═CF—CF₃ | H | Et |
| 89 | H | CF═CF—CF₃ | H | CHF—Me |
| 90 | H | CF═CF—CF₃ | H | i-Pr |
| 91 | H | CF═CF—CF₃ | H | F |
| 92 | H | CF═CF—CF₃ | H | Cl |
| 93 | H | CF═CF—CF₃ | H | Br |
| 94 | H | CF═CF—CF₃ | H | I |
| 95 | H | CF═CF—CF₃ | H | NO₂ |
| 96 | H | CF═CF—CF₃ | H | CN |
| 97 | Me | H | H | CF═CF—CF₃ |
| 98 | Et | H | H | CF═CF—CF₃ |
| 99 | CHF—Me | H | H | CF═CF—CF₃ |
| 100 | i-Pr | H | H | CF═CF—CF₃ |
| 101 | F | H | H | CF═CF—CF₃ |
| 102 | Cl | H | H | CF═CF—CF₃ |
| 103 | Br | H | H | CF═CF—CF₃ |
| 104 | I | H | H | CF═CF—CF₃ |
| 105 | NO₂ | H | H | CF═CF—CF₃ |
| 106 | CN | H | H | CF═CF—CF₃ |
| 107 | H | Me | CF═CF—CF₃ | H |
| 108 | H | Et | CF═CF—CF₃ | H |
| 109 | H | CHF—Me | CF═CF—CF₃ | H |
| 110 | H | i-Pr | CF═CF—CF₃ | H |
| 111 | H | F | CF═CF—CF₃ | H |
| 112 | H | Cl | CF═CF—CF₃ | H |
| 113 | H | Br | CF═CF—CF₃ | H |
| 114 | H | I | CF═CF—CF₃ | H |
| 115 | H | NO₂ | CF═CF—CF₃ | H |
| 116 | H | CN | CF═CF—CF₃ | H |
| 117 | H | Me | CF═CF—CF₃ | F |
| 118 | H | Me | CF═CF—CF₃ | Cl |
| 119 | H | Me | CF═CF—CF₃ | Br |
| 120 | H | Me | CF═CF—CF₃ | I |
| 121 | H | Me | CF═CF—CF₃ | Me |
| 122 | H | Et | CF═CF—CF₃ | Et |
| 123 | H | CHF—Me | CF═CF—CF₃ | CHF—Me |
| 124 | H | i-Pr | CF═CF—CF₃ | i-Pr |
| 125 | H | F | CF═CF—CF₃ | F |
| 126 | H | Cl | CF═CF—CF₃ | Cl |
| 127 | H | Br | CF═CF—CF₃ | Br |
| 128 | H | I | CF═CF—CF₃ | I |
| 129 | H | NO₂ | CF═CF—CF₃ | NO₂ |
| 130 | H | CN | CF═CF—CF₃ | CN |
| 131 | H | CF═CF—CF₃ | CF═CF—CF₃ | H |
| 132 | H | CF═CF—CF₃ | CF═CF—CF₃ | Me |
| 133 | H | CF═CF—CF₃ | CF═CF—CF₃ | Et |
| 134 | H | CF═CF—CF₃ | CF═CF—CF₃ | CHF—Me |
| 135 | H | CF═CF—CF₃ | CF═CF—CF₃ | i-Pr |
| 136 | H | CF═CF—CF₃ | CF═CF—CF₃ | F |
| 137 | H | CF═CF—CF₃ | CF═CF—CF₃ | Cl |
| 138 | H | CF═CF—CF₃ | CF═CF—CF₃ | Br |
| 139 | H | CF═CF—CF₃ | CF═CF—CF₃ | I |
| 140 | H | CF═CF—CF₃ | CF═CF—CF₃ | NO₂ |
| 141 | H | CF═CF—CF₃ | CF═CF—CF₃ | CN |
| 142 | H | CF═CF—CF₃ | H | CF═CF—CF₃ |
| 143 | H | CF═CF—CF₃ | Me | CF═CF—CF₃ |
| 144 | H | CF═CF—CF₃ | Et | CF═CF—CF₃ |
| 145 | H | CF═CF—CF₃ | CHF—Me | CF═CF—CF₃ |
| 146 | H | CF═CF—CF₃ | i-Pr | CF═CF—CF₃ |
| 147 | H | CF═CF—CF₃ | F | CF═CF—CF₃ |
| 148 | H | CF═CF—CF₃ | Cl | CF═CF—CF₃ |
| 149 | H | CF═CF—CF₃ | Br | CF═CF—CF₃ |
| 150 | H | CF═CF—CF₃ | I | CF═CF—CF₃ |
| 151 | H | CF═CF—CF₃ | NO₂ | CF═CF—CF₃ |
| 152 | H | CF═CF—CF₃ | CN | CF═CF—CF₃ |
| 153 | H | CF₂—CF═CF₂ | Me | H |
| 154 | H | CF₂—CF═CF₂ | Et | H |
| 155 | H | CF₂—CF═CF₂ | CHF—Me | H |
| 156 | H | CF₂—CF═CF₂ | i-Pr | H |
| 157 | H | CF₂—CF═CF₂ | F | H |
| 158 | H | CF₂—CF═CF₂ | Cl | H |
| 159 | H | CF₂—CF═CF₂ | Br | H |
| 160 | H | CF₂—CF═CF₂ | I | H |
| 161 | H | CF₂—CF═CF₂ | NO₂ | H |
| 162 | H | CF₂—CF═CF₂ | CN | H |
| 163 | H | CF₂—CF═CF₂ | H | Me |
| 164 | H | CF₂—CF═CF₂ | H | Et |
| 165 | H | CF₂—CF═CF₂ | H | CHF—Me |
| 166 | H | CF₂—CF═CF₂ | H | i-Pr |
| 167 | H | CF₂—CF═CF₂ | H | F |
| 168 | H | CF₂—CF═CF₂ | H | Cl |
| 169 | H | CF₂—CF═CF₂ | H | Br |
| 170 | H | CF₂—CF═CF₂ | H | I |
| 171 | H | CF₂—CF═CF₂ | H | NO₂ |
| 172 | H | CF₂—CF═CF₂ | H | CN |
| 173 | Me | H | H | CF₂—CF═CF₂ |
| 174 | Et | H | H | CF₂—CF═CF₂ |
| 175 | CHF—Me | H | H | CF₂—CF═CF₂ |
| 176 | i-Pr | H | H | CF₂—CF═CF₂ |
| 177 | F | H | H | CF₂—CF═CF₂ |
| 178 | Cl | H | H | CF₂—CF═CF₂ |
| 179 | Br | H | H | CF₂—CF═CF₂ |
| 180 | I | H | H | CF₂—CF═CF₂ |
| 181 | NO₂ | H | H | CF₂—CF═CF₂ |
| 182 | CN | H | H | CF₂—CF═CF₂ |
| 183 | H | Me | CF₂—CF═CF₂ | H |
| 184 | H | Et | CF₂—CF═CF₂ | H |
| 185 | H | CHF—Me | CF₂—CF═CF₂ | H |
| 186 | H | i-Pr | CF₂—CF═CF₂ | H |
| 187 | H | F | CF₂—CF═CF₂ | H |
| 188 | H | Cl | CF₂—CF═CF₂ | H |
| 189 | H | Br | CF₂—CF═CF₂ | H |
| 190 | H | I | CF₂—CF═CF₂ | H |
| 191 | H | NO₂ | CF₂—CF═CF₂ | H |
| 192 | H | CN | CF₂—CF═CF₂ | H |
| 193 | H | Me | CF₂—CF═CF₂ | F |
| 194 | H | Me | CF₂—CF═CF₂ | Cl |
| 195 | H | Me | CF₂—CF═CF₂ | Br |
| 196 | H | Me | CF₂—CF═CF₂ | I |
| 197 | H | Me | CF₂—CF═CF₂ | Me |
| 198 | H | Et | CF₂—CF═CF₂ | Et |
| 199 | H | CHF—Me | CF₂—CF═CF₂ | CHF—Me |
| 200 | H | i-Pr | CF₂—CF═CF₂ | i-Pr |
| 201 | H | F | CF₂—CF═CF₂ | F |
| 202 | H | Cl | CF₂—CF═CF₂ | Cl |
| 203 | H | Br | CF₂—CF═CF₂ | Br |
| 204 | H | I | CF₂—CF═CF₂ | I |
| 205 | H | NO₂ | CF₂—CF═CF₂ | NO₂ |
| 206 | H | CN | CF₂—CF═CF₂ | CN |
| 207 | H | CF₂—CF═CF₂ | CF₂—CF═CF₂ | H |
| 208 | H | CF₂—CF═CF₂ | CF₂—CF═CF₂ | Me |
| 209 | H | CF₂—CF═CF₂ | CF₂—CF═CF₂ | Et |
| 210 | H | CF₂—CF═CF₂ | CF₂—CF═CF₂ | CHF—Me |
| 211 | H | CF₂—CF═CF₂ | CF₂—CF═CF₂ | i-Pr |
| 212 | H | CF₂—CF═CF₂ | CF₂—CF═CF₂ | F |
| 213 | H | CF₂—CF═CF₂ | CF₂—CF═CF₂ | Cl |
| 214 | H | CF₂—CF═CF₂ | CF₂—CF═CF₂ | Br |
| 215 | H | CF₂—CF═CF₂ | CF₂—CF═CF₂ | I |
| 216 | H | CF₂—CF═CF₂ | CF₂—CF═CF₂ | NO₂ |
| 217 | H | CF₂—CF═CF₂ | CF₂—CF═CF₂ | CN |
| 218 | H | CF₂—CF═CF₂ | H | CF₂—CF═CF₂ |
| 219 | H | CF₂—CF═CF₂ | Me | CF₂—CF═CF₂ |
| 220 | H | CF₂—CF═CF₂ | Et | CF₂—CF═CF₂ |
| 221 | H | CF₂—CF═CF₂ | CHF—Me | CF₂—CF═CF₂ |
| 222 | H | CF₂—CF═CF₂ | i-Pr | CF₂—CF═CF₂ |
| 223 | H | CF₂—CF═CF₂ | F | CF₂—CF═CF₂ |
| 224 | H | CF₂—CF═CF₂ | Cl | CF₂—CF═CF₂ |
| 225 | H | CF₂—CF═CF₂ | Br | CF₂—CF═CF₂ |
| 226 | H | CF₂—CF═CF₂ | I | CF₂—CF═CF₂ |
| 227 | H | CF₂—CF═CF₂ | NO₂ | CF₂—CF═CF₂ |
| 228 | H | CF₂—CF═CF₂ | CN | CF₂—CF═CF₂ |
| 229 | H | CF₂—CF═CF—CF₃ | Me | H |
| 230 | H | CF₂—CF═CF—CF₃ | Et | H |
| 231 | H | CF₂—CF═CF—CF₃ | CHF—Me | H |
| 232 | H | CF₂—CF═CF—CF₃ | i-Pr | H |
| 233 | H | CF₂—CF═CF—CF₃ | F | H |
| 234 | H | CF₂—CF═CF—CF₃ | Cl | H |
| 235 | H | CF₂—CF═CF—CF₃ | Br | H |
| 236 | H | CF₂—CF═CF—CF₃ | I | H |
| 237 | H | CF₂—CF═CF—CF₃ | NO₂ | H |
| 238 | H | CF₂—CF═CF—CF₃ | CN | H |
| 239 | H | CF₂—CF═CF—CF₃ | H | Me |
| 240 | H | CF₂—CF═CF—CF₃ | H | Et |
| 241 | H | CF₂—CF═CF—CF₃ | H | CHF—Me |
| 242 | H | CF₂—CF═CF—CF₃ | H | i-Pr |
| 243 | H | CF₂—CF═CF—CF₃ | H | F |
| 244 | H | CF₂—CF═CF—CF₃ | H | Cl |
| 245 | H | CF₂—CF═CF—CF₃ | H | Br |
| 246 | H | CF₂—CF═CF—CF₃ | H | I |
| 247 | H | CF₂—CF═CF—CF₃ | H | NO₂ |
| 248 | H | CF₂—CF═CF—CF₃ | H | CN |
| 249 | Me | H | H | CF₂—CF═CF—CF₃ |
| 250 | Et | H | H | CF₂—CF═CF—CF₃ |
| 251 | CHF—Me | H | H | CF₂—CF═CF—CF₃ |
| 252 | i-Pr | H | H | CF₂—CF═CF—CF₃ |
| 253 | F | H | H | CF₂—CF═CF—CF₃ |
| 254 | Cl | H | H | CF₂—CF═CF—CF₃ |
| 255 | Br | H | H | CF₂—CF═CF—CF₃ |
| 256 | I | H | H | CF₂—CF═CF—CF₃ |
| 257 | NO₂ | H | H | CF₂—CF═CF—CF₃ |
| 258 | CN | H | H | CF₂—CF═CF—CF₃ |
| 259 | H | Me | CF₂—CF═CF—CF₃ | H |
| 260 | H | Et | CF₂—CF═CF—CF₃ | H |
| 261 | H | CHF—Me | CF₂—CF═CF—CF₃ | H |
| 262 | H | i-Pr | CF₂—CF═CF—CF₃ | H |
| 263 | H | F | CF₂—CF═CF—CF₃ | H |
| 264 | H | Cl | CF₂—CF═CF—CF₃ | H |
| 265 | H | Br | CF₂—CF═CF—CF₃ | H |
| 266 | H | I | CF₂—CF═CF—CF₃ | H |
| 267 | H | NO₂ | CF₂—CF═CF—CF₃ | H |
| 268 | H | CN | CF₂—CF═CF—CF₃ | H |
| 269 | H | Me | CF₂—CF═CF—CF₃ | F |
| 270 | H | Me | CF₂—CF═CF—CF₃ | Cl |
| 271 | H | Me | CF₂—CF═CF—CF₃ | Br |
| 272 | H | Me | CF₂—CF═CF—CF₃ | I |
| 273 | H | Me | CF₂—CF═CF—CF₃ | Me |
| 274 | H | Et | CF₂—CF═CF—CF₃ | Et |
| 275 | H | CHF—Me | CF₂—CF═CF—CF₃ | CHF—Me |
| 276 | H | i-Pr | CF₂—CF═CF—CF₃ | i-Pr |
| 277 | H | F | CF₂—CF═CF—CF₃ | F |
| 278 | H | Cl | CF₂—CF═CF—CF₃ | Cl |
| 279 | H | Br | CF₂—CF═CF—CF₃ | Br |
| 280 | H | I | CF₂—CF═CF—CF₃ | I |
| 281 | H | NO₂ | CF₂—CF═CF—CF₃ | NO₂ |
| 282 | H | CN | CF₂—CF═CF—CF₃ | CN |
| 283 | H | CF₂—CF═CF—CF₃ | CF₂—CF═CF—CF₃ | H |
| 284 | H | CF₂—CF═CF—CF₃ | CF₂—CF═CF—CF₃ | Me |
| 285 | H | CF₂—CF═CF—CF₃ | CF₂—CF═CF—CF₃ | Et |
| 286 | H | CF₂—CF═CF—CF₃ | CF₂—CF═CF—CF₃ | CHF—Me |
| 287 | H | CF₂—CF═CF—CF₃ | CF₂—CF═CF—CF₃ | i-Pr |
| 288 | H | CF₂—CF═CF—CF₃ | CF₂—CF═CF—CF₃ | F |
| 289 | H | CF₂—CF═CF—CF₃ | CF₂—CF═CF—CF₃ | Cl |
| 290 | H | CF₂—CF═CF—CF₃ | CF₂—CF═CF—CF₃ | Br |
| 291 | H | CF₂—CF═CF—CF₃ | CF₂—CF═CF—CF₃ | I |
| 292 | H | CF₂—CF═CF—CF₃ | CF₂—CF═CF—CF₃ | NO₂ |
| 293 | H | CF₂—CF═CF—CF₃ | CF₂—CF═CF—CF₃ | CN |
| 294 | H | CF₂—CF═CF—CF₃ | H | CF₂—CF═CF—CF₃ |
| 295 | H | CF₂—CF═CF—CF₃ | Me | CF₂—CF═CF—CF₃ |
| 296 | H | CF₂—CF═CF—CF₃ | Et | CF₂—CF═CF—CF₃ |
| 297 | H | CF₂—CF═CF—CF₃ | CHF—Me | CF₂—CF═CF—CF₃ |
| 298 | H | CF₂—CF═CF—CF₃ | i-Pr | CF₂—CF═CF-CF₃ |
| 299 | H | CF₂—CF═CF—CF₃ | F | CF₂—CF═CF—CF₃ |
| 300 | H | CF₂—CF═CF—CF₃ | Cl | CF₂—CF═CF—CF₃ |
| 301 | H | CF₂—CF═CF—CF₃ | Br | CF₂—CF═CF—CF₃ |
| 302 | H | CF₂—CF═CF—CF₃ | I | CF₂—CF═CF—CF₃ |
| 303 | H | CF₂—CF═CF—CF₃ | NO₂ | CF₂—CF═CF—CF₃ |
| 304 | H | CF₂—CF═CF—CF₃ | CN | CF₂—CF═CF—CF₃ |
| 305 | H | CCH | Me | H |
| 308 | H | CCH | Et | H |
| 307 | H | CCH | CHF—Me | H |
| 308 | H | CCH | i-Pr | H |
| 309 | H | CCH | F | H |
| 310 | H | CCH | Cl | H |
| 311 | H | CCH | Br | H |
| 312 | H | CCH | I | H |
| 313 | H | CCH | NO₂ | H |
| 314 | H | CCH | CN | H |
| 315 | H | CCH | H | Me |
| 316 | H | CCH | H | Et |
| 317 | H | CCH | H | CHF—Me |
| 318 | H | CCH | H | i-Pr |
| 319 | H | CCH | H | F |
| 320 | H | CCH | H | Cl |
| 321 | H | CCH | H | Br |
| 322 | H | CCH | H | I |
| 323 | H | CCH | H | NO₂ |
| 324 | H | CCH | H | CN |
| 325 | Me | H | H | CCH |
| 326 | Et | H | H | CCH |
| 327 | CHF—Me | H | H | CCH |
| 328 | i-Pr | H | H | CCH |
| 329 | F | H | H | CCH |
| 330 | Cl | H | H | CCH |
| 331 | Br | H | H | CCH |
| 332 | I | H | H | CCH |
| 333 | NO₂ | H | H | CCH |
| 334 | CN | H | H | CCH |
| 335 | H | Me | CCH | H |
| 336 | H | Et | CCH | H |
| 337 | H | CHF—Me | CCH | H |
| 338 | H | i-Pr | CCH | H |
| 339 | H | Cl | CCH | H |
| 340 | H | Br | CCH | H |
| 341 | H | I | CCH | H |
| 342 | H | NO₂ | CCH | H |
| 343 | H | CN | CCH | H |
| 344 | H | Me | CCH | F |
| 345 | H | Me | CCH | Cl |
| 346 | H | Me | CCH | Br |
| 347 | H | Me | CCH | I |
| 348 | H | Me | CCH | Me |
| 349 | H | Et | CCH | Et |
| 350 | H | CHF—Me | CCH | CHF—Me |
| 351 | H | i-Pr | CCH | i-Pr |
| 352 | H | F | CCH | F |
| 353 | H | Cl | CCH | Cl |
| 354 | H | Br | CCH | Br |
| 355 | H | I | CCH | I |
| 356 | H | NO₂ | CCH | NO₂ |
| 357 | H | CN | CCH | CN |
| 358 | H | CCH | CCH | H |
| 359 | H | CCH | CCH | Me |
| 360 | H | CCH | CCH | Et |
| 361 | H | CCH | CCH | CHF—Me |
| 362 | H | CCH | CCH | i-Pr |
| 363 | H | CCH | CCH | F |
| 364 | H | CCH | CCH | Cl |
| 365 | H | CCH | CCH | Br |
| 366 | H | CCH | CCH | I |
| 367 | H | CCH | CCH | NO₂ |
| 368 | H | CCH | CCH | CN |
| 369 | H | CCH | H | CCH |
| 370 | H | CCH | Me | CCH |
| 371 | H | CCH | Et | CCH |
| 372 | H | CCH | CHF—Me | CCH |
| 373 | H | CCH | i-Pr | CCH |
| 374 | H | CCH | F | CCH |
| 375 | H | CCH | Cl | CCH |
| 376 | H | CCH | Br | CCH |
| 377 | H | CCH | I | CCH |
| 378 | H | CCH | NO₂ | CCH |
| 379 | H | CCH | CN | CCH |
| 380 | H | CCMe | Me | H |
| 381 | H | CCMe | Et | H |
| 382 | H | CCMe | CHF—Me | H |
| 383 | H | CCMe | i-Pr | H |
| 384 | H | CCMe | F | H |
| 385 | H | CCMe | Cl | H |
| 386 | H | CCMe | Br | H |
| 387 | H | CCMe | I | H |
| 388 | H | CCMe | NO₂ | H |
| 389 | H | CCMe | CN | H |
| 390 | H | CCMe | H | Me |
| 391 | H | CCMe | H | Et |
| 392 | H | CCMe | H | CHF—Me |
| 393 | H | CCMe | H | i-Pr |
| 394 | H | CCMe | H | F |
| 395 | H | CCMe | H | Cl |
| 396 | H | CCMe | H | Br |
| 397 | H | CCMe | H | I |
| 398 | H | CCMe | H | NO₂ |
| 399 | H | CCMe | H | CN |
| 400 | Me | H | H | CCMe |
| 401 | Et | H | H | CCMe |
| 402 | CHF—Me | H | H | CCMe |
| 403 | i-Pr | H | H | CCMe |
| 404 | F | H | H | CCMe |
| 405 | Cl | H | H | CCMe |
| 406 | Br | H | H | CCMe |
| 407 | I | H | H | CCMe |
| 408 | NO₂ | H | H | CCMe |
| 409 | CN | H | H | CCMe |
| 410 | H | Me | CCMe | H |
| 411 | H | Et | CCMe | H |
| 412 | H | CHF—Me | CCMe | H |
| 413 | H | i-Pr | CCMe | H |
| 414 | H | F | CCMe | H |
| 415 | H | Cl | CCMe | H |
| 416 | H | Br | CCMe | H |
| 417 | H | I | CCMe | H |
| 418 | H | NO₂ | CCMe | H |
| 419 | H | CN | CCMe | H |
| 420 | H | Me | CCMe | F |
| 421 | H | Me | CCMe | Cl |
| 422 | H | Me | CCMe | Br |
| 423 | H | Me | CCMe | I |
| 424 | H | Me | CCMe | Me |
| 425 | H | Et | CCMe | Et |
| 426 | H | CHF—Me | CCMe | CHF—Me |
| 427 | H | i-Pr | CCMe | i-Pr |
| 428 | H | F | CCMe | F |
| 429 | H | Cl | CCMe | Cl |
| 430 | H | Br | CCMe | Br |
| 431 | H | I | CCMe | I |
| 432 | H | NO₂ | CCMe | NO₂ |
| 433 | H | CN | CCMe | CN |
| 434 | H | CCMe | CCMe | H |
| 435 | H | CCMe | CCMe | Me |
| 436 | H | CCMe | CCMe | Et |
| 437 | H | CCMe | CCMe | CHF—Me |
| 438 | H | CCMe | CCMe | i-Pr |
| 439 | H | CCMe | CCMe | F |
| 440 | H | CCMe | CCMe | Cl |
| 441 | H | CCMe | CCMe | Br |
| 442 | H | CCMe | CCMe | I |
| 443 | H | CCMe | CCMe | NO₂ |
| 444 | H | CCMe | CCMe | CN |
| 445 | H | CCMe | H | CCMe |
| 446 | H | CCMe | Me | CCMe |
| 447 | H | CCMe | Et | CCMe |
| 448 | H | CCMe | CHF—Me | CCMe |
| 440 | H | CCMe | i-Pr | CCMe |
| 460 | H | CCMe | F | CCMe |
| 451 | H | CCMe | Cl | CCMe |
| 452 | H | CCMe | Br | CCMe |
| 453 | H | CCMe | I | CCMe |
| 454 | H | CCMe | NO₂ | CCMe |
| 455 | H | CCMe | CN | CCMe |
| 456 | H | CH₂CCH | Me | H |
| 457 | H | CH₂CCH | Et | H |
| 458 | H | CH₂CCH | CHF—Me | H |
| 459 | H | CH₂CCH | i-Pr | H |
| 460 | H | CH₂CCH | F | H |
| 461 | H | CH₂CCH | Cl | H |
| 462 | H | CH₂CCH | Br | H |
| 463 | H | CH₂CCH | I | H |
| 464 | H | CH₂CCH | NO₂ | H |
| 465 | H | CH₂CCH | CN | H |
| 466 | H | CH₂CCH | H | Me |
| 467 | H | CH₂CCH | H | Et |
| 468 | H | CH₂CCH | H | CHF—Me |
| 469 | H | CH₂CCH | H | i-Pr |
| 470 | H | CH₂CCH | H | F |
| 471 | H | CH₂CCH | H | Cl |
| 472 | H | CH₂CCH | H | Br |
| 473 | H | CH₂CCH | H | I |
| 474 | H | CH₂CCH | H | NO₂ |
| 475 | H | CH₂CCH | H | CN |
| 476 | Me | H | H | CH₂CCH |
| 477 | Et | H | H | CH₂CCH |
| 478 | CHF—Me | H | H | CH₂CCH |
| 479 | i-Pr | H | H | CH₂CCH |
| 480 | F | H | H | CH₂CCH |
| 481 | Cl | H | H | CH₂CCH |
| 482 | Br | H | H | CH₂CCH |
| 483 | I | H | H | CH₂CCH |
| 484 | NO₂ | H | H | CH₂CCH |
| 485 | CN | H | H | CH₂CCH |
| 486 | H | Me | CH₂CCH | H |
| 487 | H | Et | CH₂CCH | H |
| 488 | H | CHF—Me | CH₂CCH | H |
| 489 | H | i-Pr | CH₂CCH | H |
| 490 | H | F | CH₂CCH | H |
| 491 | H | Cl | CH₂CCH | H |
| 492 | H | Br | CH₂CCH | H |
| 493 | H | I | CH₂CCH | H |
| 494 | H | NO₂ | CH₂CCH | H |
| 495 | H | CN | CH₂CCH | H |
| 496 | H | Me | CH₂CCH | F |
| 497 | H | Me | CH₂CCH | Cl |
| 498 | H | Me | CH₂CCH | Br |
| 499 | H | Me | CH₂CCH | I |
| 500 | H | Me | CH₂CCH | Me |
| 501 | H | Et | CH₂CCH | Et |
| 502 | H | CHF—Me | CH₂CCH | CHF—Me |
| 503 | H | i-Pr | CH₂CCH | i-Pr |
| 504 | H | F | CH₂CCH | F |
| 505 | H | Cl | CH₂CCH | Cl |
| 506 | H | Br | CH₂CCH | Br |
| 507 | H | I | CH₂CCH | I |
| 508 | H | NO₂ | CH₂CCH | NO₂ |
| 509 | H | CN | CH₂CCH | CN |
| 510 | H | CH₂CCH | CH₂CCH | H |
| 511 | H | CH₂CCH | CH₂CCH | Me |
| 512 | H | CH₂CCH | CH₂CCH | Et |
| 513 | H | CH₂CCH | CH₂CCH | CHF—Me |
| 514 | H | CH₂CCH | CH₂CCH | i-Pr |
| 515 | H | CH₂CCH | CH₂CCH | F |
| 516 | H | CH₂CCH | CH₂CCH | Cl |
| 517 | H | CH₂CCH | CH₂CCH | Br |
| 518 | H | CH₂CCH | CH₂CCH | I |
| 519 | H | CH₂CCH | CH₂CCH | NO₂ |
| 520 | H | CH₂CCH | CH₂CCH | CN |
| 521 | H | CH₂CCH | H | CH₂CCH |
| 522 | H | CH₂CCH | Me | CH₂CCH |
| 523 | H | CH₂CCH | Et | CH₂CCH |
| 524 | H | CH₂CCH | CHF—Me | CH₂CCH |
| 525 | H | CH₂CCH | i-Pr | CH₂CCH |
| 526 | H | CH₂CCH | F | CH₂CCH |
| 527 | H | CH₂CCH | Cl | CH₂CCH |
| 528 | H | CH₂CCH | Br | CH₂CCH |
| 529 | H | CH₂CCH | I | CH₂CCH |
| 530 | H | CH₂CCH | NO₂ | CH₂CCH |
| 531 | H | CH₂CCH | CN | CH₂CCH |
| 532 | H | CH₂CCMe | Me | H |
| 533 | H | CH₂CCMe | Et | H |
| 534 | H | CH₂CCMe | CHF—Me | H |
| 535 | H | CH₂CCMe | i-Pr | H |
| 538 | H | CH₂CCMe | F | H |
| 537 | H | CH₂CCMe | Cl | H |
| 538 | H | CH₂CCMe | Br | H |
| 539 | H | CH₂CCMe | I | H |
| 540 | H | CH₂CCMe | NO₂ | H |
| 541 | H | CH₂CCMe | CN | H |
| 542 | H | CH₂CCMe | H | Me |
| 543 | H | CH₂CCMe | H | Et |
| 544 | H | CH₂CCMe | H | CHF—Me |
| 545 | H | CH₂CCMe | H | i-Pr |
| 546 | H | CH₂CCMe | H | F |
| 547 | H | CH₂CCMe | H | Cl |
| 548 | H | CH₂CCMe | H | Br |
| 549 | H | CH₂CCMe | H | I |
| 550 | H | CH₂CCMe | H | NO₂ |
| 551 | H | CH₂CCMe | H | CN |
| 552 | Me | H | H | CH₂CCMe |
| 553 | Et | H | H | CH₂CCMe |
| 554 | CHF—Me | H | H | CH₂CCMe |
| 555 | i-Pr | H | H | CH₂CCMe |
| 556 | F | H | H | CH₂CCMe |
| 557 | Cl | H | H | CH₂CCMe |
| 558 | Br | H | H | CH₂CCMe |
| 559 | I | H | H | CH₂CCMe |
| 560 | NO₂ | H | H | CH₂CCMe |
| 561 | CN | H | H | CH₂CCMe |
| 562 | H | Me | CH₂CCMe | H |
| 563 | H | Et | CH₂CCMe | H |
| 564 | H | CHF—Me | CH₂CCMe | H |
| 565 | H | i-Pr | CH₂CCMe | H |
| 566 | H | F | CH₂CCMe | H |
| 567 | H | Cl | CH₂CCMe | H |
| 568 | H | Br | CH₂CCMe | H |
| 569 | H | I | CH₂CCMe | H |
| 570 | H | NO₂ | CH₂CCMe | H |
| 571 | H | CN | CH₂CCMe | H |
| 572 | H | Me | CH₂CCMe | F |
| 573 | H | Me | CH₂CCMe | Cl |
| 574 | H | Me | CH₂CCMe | Br |
| 575 | H | Me | CH₂CCMe | I |
| 576 | H | Me | CH₂CCMe | Me |
| 577 | H | Et | CH₂CCMe | Et |
| 578 | H | CHF—Me | CH₂CCMe | CHF—Me |
| 579 | H | i-Pr | CH₂CCMe | i-Pr |
| 580 | H | F | CH₂CCMe | F |
| 581 | H | Cl | CH₂CCMe | Cl |
| 582 | H | Br | CH₂CCMe | Br |
| 583 | H | I | CH₂CCMe | I |
| 584 | H | NO₂ | CH₂CCMe | NO₂ |
| 585 | H | CN | CH₂CCMe | CN |
| 586 | H | CH₂CCMe | CH₂CCMe | H |
| 587 | H | CH₂CCMe | CH₂CCMe | Me |
| 588 | H | CH₂CCMe | CH₂CCMe | Et |
| 589 | H | CH₂CCMe | CH₂CCMe | CHF—Me |
| 590 | H | CH₂CCMe | CH₂CCMe | i-Pr |
| 591 | H | CH₂CCMe | CH₂CCMe | F |
| 592 | H | CH₂CCMe | CH₂CCMe | Cl |
| 593 | H | CH₂CCMe | CH₂CCMe | Br |
| 594 | H | CH₂CCMe | CH₂CCMe | I |
| 595 | H | CH₂CCMe | CH₂CCMe | NO₂ |
| 596 | H | CH₂CCMe | CH₂CCMe | CN |
| 597 | H | CH₂CCMe | H | CH₂CCMe |
| 598 | H | CH₂CCMe | Me | CH₂CCMe |
| 599 | H | CH₂CCMe | Et | CH₂CCMe |
| 600 | H | CH₂CCMe | CHF—Me | CH₂CCMe |
| 601 | H | CH₂CCMe | i-Pr | CH₂CCMe |
| 602 | H | CH₂CCMe | F | CH₂CCMe |
| 603 | H | CH₂CCMe | Cl | CH₂CCMe |
| 604 | H | CH₂CCMe | Br | CH₂CCMe |
| 605 | H | CH₂CCMe | I | CH₂CCMe |
| 606 | H | CH₂CCMe | NO₂ | CH₂CCMe |
| 607 | H | CH₂CCMe | CN | CH₂CCMe |
| 608 | H | CH═C═CH₂ | Me | H |
| 609 | H | CH═C═CH₂ | Et | H |
| 610 | H | CH═C═CH₂ | CHF—Me | H |
| 611 | H | CH═C═CH₂ | i-Pr | H |
| 612 | H | CH═C═CH₂ | F | H |
| 613 | H | CH═C═CH₂ | Cl | H |
| 614 | H | CH═C═CH₂ | Br | H |
| 615 | H | CH═C═CH₂ | I | H |
| 616 | H | CH═C═CH₂ | NO₂ | H |
| 617 | H | CH═C═CH₂ | CN | H |
| 618 | H | CH═C═CH₂ | H | Me |
| 619 | H | CH═C═CH₂ | H | Et |
| 620 | H | CH═C═CH₂ | H | CHF—Me |
| 621 | H | CH═C═CH₂ | H | i-Pr |
| 622 | H | CH═C═CH₂ | H | F |
| 623 | H | CH═C═CH₂ | H | Cl |
| 624 | H | CH═C═CH₂ | H | Br |
| 625 | H | CH═C═CH₂ | H | I |
| 626 | H | CH═C═CH₂ | H | NO₂ |
| 627 | H | CH═C═CH₂ | H | CN |
| 628 | Me | H | H | CH═C═CH₂ |
| 629 | Et | H | H | CH═C═CH₂ |
| 630 | CHF—Me | H | H | CH═C═CH₂ |
| 631 | i-Pr | H | H | CH═C═CH₂ |
| 632 | F | H | H | CH═C═CH₂ |
| 633 | Cl | H | H | CH═C═CH₂ |
| 634 | Br | H | H | CH═C═CH₂ |
| 635 | I | H | H | CH═C═CH₂ |
| 636 | NO₂ | H | H | CH═C═CH₂ |
| 637 | CN | H | H | CH═C═CH₂ |
| 638 | H | Me | CH═C═CH₂ | H |
| 639 | H | Et | CH═C═CH₂ | H |
| 640 | H | CHF—Me | CH═C═CH₂ | H |
| 641 | H | i-Pr | CH═C═CH₂ | H |
| 642 | H | F | CH═C═CH₂ | H |
| 643 | H | Cl | CH═C═CH₂ | H |
| 644 | H | Br | CH═C═CH₂ | H |
| 645 | H | I | CH═C═CH₂ | H |
| 646 | H | NO₂ | CH═C═CH₂ | H |
| 647 | H | CN | CH═C═CH₂ | H |
| 648 | H | Me | CH═CCH₂ | F |
| 649 | H | Me | CH═C═CH₂ | Cl |
| 650 | H | Me | CH═C═CH₂ | Br |
| 651 | H | Me | CH═C═CH₂ | I |
| 652 | H | Me | CH═C═CH₂ | Me |
| 653 | H | Et | CH═C═CH₂ | Et |
| 654 | H | CHF—Me | CH═C═CH₂ | CHF—Me |
| 655 | H | i-Pr | CH═C═CH₂ | i-Pr |
| 656 | H | F | CH═C═CH₂ | F |
| 657 | H | Cl | CH═C═CH₂ | Cl |
| 658 | H | Br | CH═C═CH₂ | Br |
| 659 | H | I | CH═C═CH₂ | I |
| 660 | H | NO₂ | CH═C═CH₂ | NO₂ |
| 661 | H | CN | CH═C═CH₂ | CN |
| 662 | H | CH═C═CH₂ | CH═C═CH₂ | H |
| 663 | H | CH═C═CH₂ | CH═C═CH₂ | Me |
| 664 | H | CH═C═CH₂ | CH═C═CH₂ | Et |
| 665 | H | CH═C═CH₂ | CH═C═CH₂ | CHF—Me |
| 666 | H | CH═C═CH₂ | CH═C═CH₂ | i-Pr |
| 667 | H | CH═C═CH₂ | CH═C═CH₂ | F |
| 668 | H | CH═C═CH₂ | CH═C═CH₂ | Cl |
| 669 | H | CH═C═CH₂ | CH═C═CH₂ | Br |
| 670 | H | CH═C═CH₂ | CH═C═CH₂ | I |
| 671 | H | CH═C═CH₂ | CH═C═CH₂ | NO₂ |
| 672 | H | CH═C═CH₂ | CH═C═CH₂ | CN |
| 673 | H | CH═C═CH₂ | H | CH═C═CH₂ |
| 674 | H | CH═C═CH₂ | Me | CH═C═CH₂ |
| 675 | H | CH═C═CH₂ | Et | CH═C═CH₂ |
| 676 | H | CH═C═CH₂ | CHF—Me | CH═C═CH₂ |
| 677 | H | CH═C═CH₂ | i-Pr | CH═C═CH₂ |
| 678 | H | CH═C═CH₂ | F | CH═C═CH₂ |
| 679 | H | CH═C═CH₂ | Cl | CH═C═CH₂ |
| 680 | H | CH═C═CH₂ | Br | CH═C═CH₂ |
| 681 | H | CH═C═CH₂ | I | CH═C═CH₂ |
| 682 | H | CH═C═CH₂ | NO₂ | CH═C═CH₂ |
| 683 | H | CH═C═CH₂ | CN | CH═C═CH₂ |
| 684 | H | CC-Bu | Me | H |
| 685 | H | CC-Bu | Et | H |
| 686 | H | CC-Bu | CHF—Me | H |
| 687 | H | CC-Bu | i-Pr | H |
| 688 | H | CC-Bu | F | H |
| 689 | H | CC-Bu | Cl | H |
| 690 | H | CC-Bu | Br | H |
| 691 | H | CC-BU | I | H |
| 692 | H | CC-Bu | NO₂ | H |
| 693 | H | CC-Bu | CN | H |
| 694 | H | CC-Bu | H | Me |
| 695 | H | CC-Bu | H | Et |
| 696 | H | CC-Bu | H | CHF—Me |
| 697 | H | CC-Bu | H | i-Pr |
| 698 | H | CC-Bu | H | F |
| 699 | H | CC-Bu | H | Cl |
| 700 | H | CC-Bu | H | Br |
| 701 | H | CC-Bu | H | I |
| 702 | H | CC-Bu | H | NO₂ |
| 703 | H | CC-Bu | H | CN |
| 704 | Me | H | H | CC-Bu |
| 705 | Et | H | H | CC-Bu |
| 706 | CHF—Me | H | H | CC-Bu |
| 707 | i-Pr | H | H | CC-Bu |
| 708 | F | H | H | CC-Bu |
| 709 | Cl | H | H | CC-Bu |
| 710 | Br | H | H | CC-Bu |
| 711 | I | H | H | CC-Bu |
| 712 | NO₂ | H | H | CC-Bu |
| 713 | CN | H | H | CC-Bu |
| 714 | H | Me | CC-Bu | H |
| 715 | H | Et | CC-Bu | H |
| 716 | H | CHF—Me | CC-Bu | H |
| 717 | H | i-Pr | CC-Bu | H |
| 718 | H | F | CC-Bu | H |
| 719 | H | Cl | CC-Bu | H |
| 720 | H | Br | CC-Bu | H |
| 721 | H | I | CC-Bu | H |
| 722 | H | NO₂ | CC-Bu | H |
| 723 | H | CN | CC-Bu | H |
| 724 | H | Me | CC-Bu | F |
| 725 | H | Me | CC-Bu | Cl |
| 726 | H | Me | CC-Bu | Br |
| 727 | H | Me | CC-Bu | I |
| 728 | H | Me | CC-Bu | Me |
| 729 | H | Et | CC-Bu | Et |
| 730 | H | CHF—Me | CC-Bu | CHF—Me |
| 731 | H | i-Pr | CC-Bu | i-Pr |
| 732 | H | F | CC-Bu | F |
| 733 | H | Cl | CC-Bu | Cl |
| 734 | H | Br | CC-Bu | Br |
| 735 | H | I | CC-Bu | I |
| 736 | H | NO₂ | CC-Bu | NO₂ |
| 737 | H | CN | CC-Bu | CN |
| 738 | H | CC-Bu | CC-Bu | H |
| 739 | H | CC-Bu | CC-Bu | Me |
| 740 | H | CC-Bu | CC-Bu | Et |
| 741 | H | CC-Bu | CC-Bu | CHF—Me |
| 742 | H | CC-Bu | CC-Bu | i-Pr |
| 743 | H | CC-Bu | CC-Bu | F |
| 744 | H | CC-Bu | CC-Bu | Cl |
| 745 | H | CC-Bu | CC-Bu | Br |
| 746 | H | CC-Bu | CC-Bu | I |
| 747 | H | CC-Bu | CC-Bu | NO₂ |
| 748 | H | CC-Bu | CC-Bu | CN |
| 749 | H | CC-Bu | H | CC-Bu |
| 750 | H | CC-Bu | Me | CC-Bu |
| 751 | H | CC-Bu | Et | CC-Bu |
| 752 | H | CC-Bu | CHF—Me | CC-Bu |
| 753 | H | CC-Bu | i-Pr | CC-Bu |
| 754 | H | CC-Bu | F | CC-Bu |
| 755 | H | CC-Bu | Cl | CC-Bu |
| 756 | H | CC-Bu | Br | CC-Bu |
| 757 | H | CC-Bu | I | CC-Bu |
| 758 | H | CC-Bu | NO₂ | CC-Bu |
| 759 | H | CC-Bu | CN | CC-Bu |
| 760 | H | CC—CHF—Me | Me | H |
| 761 | H | CC—CHF—Me | Et | H |
| 762 | H | CC—CHF—Me | CHF—Me | H |
| 763 | H | CC—CHF—Me | i-Pr | H |
| 764 | H | CC—CHF—Me | F | H |
| 765 | H | CC—CHF—Me | Cl | H |
| 766 | H | CC—CHF—Me | Br | H |
| 767 | H | CC—CHF—Me | I | H |
| 768 | H | CC—CHF—Me | NO₂ | H |
| 769 | H | CC—CHF—Me | CN | H |
| 770 | H | CC—CHF—Me | H | Me |
| 771 | H | CC—CHF—Me | H | Et |
| 772 | H | CC—CHF—Me | H | CHF—Me |
| 773 | H | CC—CHF—Me | H | i-Pr |
| 774 | H | CC—CHF—Me | H | F |
| 775 | H | CC—CHF—Me | H | Cl |
| 776 | H | CC—CHF—Me | H | Br |
| 777 | H | CC—CHF—Me | H | I |
| 778 | H | CC—CHF—Me | H | NO₂ |
| 779 | H | CC—CHF—Me | H | CN |
| 780 | Me | H | H | CC—CHF—Me |
| 781 | Et | H | H | CC—CHF—Me |
| 782 | CHF—Me | H | H | CC—CHF—Me |
| 783 | i-Pr | H | H | CC—CHF—Me |
| 784 | F | H | H | CC—CHF—Me |
| 785 | Cl | H | H | CC—CHF—Me |
| 786 | Br | H | H | CC—CHF—Me |
| 787 | I | H | H | CC—CHF—Me |
| 788 | NO₂ | H | H | CC—CHF—Me |
| 789 | CN | H | H | CC—CHF—Me |
| 790 | H | Me | CC—CHF—Me | H |
| 791 | H | Et | CC—CHF—Me | H |
| 792 | H | CHF—Me | CC—CHF—Me | H |
| 793 | H | i-Pr | CC—CHF—Me | H |
| 794 | H | F | CC—CHF—Me | H |
| 795 | H | Cl | CC—CHF—Me | H |
| 796 | H | Br | CC—CHF—Me | H |
| 797 | H | I | CC—CHF—Me | H |
| 798 | H | NO₂ | CC—CHF—Me | H |
| 799 | H | CN | CC—CHF—Me | H |
| 800 | H | Me | CC—CHF—Me | F |
| 801 | H | Me | CC—CHF—Me | Cl |
| 802 | H | Me | CC—CHF—Me | Br |
| 803 | H | Me | CC—CHF—Me | I |
| 804 | H | Me | CC—CHF—Me | Me |
| 805 | H | Et | CC—CHF—Me | Et |
| 806 | H | CHF—Me | CC—CHF—Me | CHF—Me |
| 807 | H | i-Pr | CC—CHF—Me | i-Pr |
| 808 | H | F | CC—CHF—Me | F |
| 809 | H | Cl | CC—CHF—Me | Cl |
| 810 | H | Br | CC—CHF—Me | Br |
| 811 | H | I | CC—CHF—Me | I |
| 812 | H | NO₂ | CC—CHF—Me | NO₂ |
| 813 | H | CN | CC—CHF—Me | CN |
| 814 | H | CC—CHF—Me | CC—CHF—Me | H |
| 815 | H | CC—CHF—Me | CC—CHF—Me | Me |
| 816 | H | CC—CHF—Me | CC—CHF—Me | Et |
| 817 | H | CC—CHF—Me | CC—CHF—Me | CHF—Me |
| 818 | H | CC—CHF—Me | CC—CHF—Me | i-Pr |
| 819 | H | CC—CHF—Me | CC—CHF—Me | F |
| 820 | H | CC—CHF—Me | CC—CHF—Me | Cl |
| 821 | H | CC—CHF—Me | CC—CHF—Me | Br |
| 822 | H | CC—CHF—Me | CC—CHF—Me | I |
| 823 | H | CC—CHF—Me | CC—CHF—Me | NO₂ |
| 824 | H | CC—CHF—Me | CC—CHF—Me | CN |
| 825 | H | CC—CHF—Me | H | CC—CHF—Me |
| 826 | H | CC—CHF—Me | Me | CC—CHF—Me |
| 827 | H | CC—CHF—Me | Et | CC—CHF—Me |
| 828 | H | CC—CHF—Me | CHF—Me | CC—CHF—Me |
| 829 | H | CC—CHF—Me | i-Pr | CC—CHF—Me |
| 830 | H | CC—CHF—Me | F | CC—CHF—Me |
| 831 | H | CC—CHF—Me | Cl | CC—CHF—Me |
| 832 | H | CC—CHF—Me | Br | CC—CHF—Me |
| 833 | H | CC—CHF—Me | I | CC—CHF—Me |
| 834 | H | CC—CHF—Me | NO₂ | CC—CHF—Me |
| 835 | H | CC—CHF—Me | CN | CC—CHF—Me |
| 836 | H | CC—CH(OH)—Me | Me | H |
| 837 | H | CC—CH(OH)—Me | Et | H |
| 838 | H | CC—CH(OH)—Me | CHF—Me | H |
| 839 | H | CC—CH(OH)—Me | i-Pr | H |
| 840 | H | CC—CH(OH)—Me | F | H |
| 841 | H | CC—CH(OH)—Me | Cl | H |
| 842 | H | CC—CH(OH)—Me | Br | H |
| 843 | H | CC—CH(OH)—Me | I | H |
| 844 | H | CC—CH(OH)—Me | NO₂ | H |
| 845 | H | CC—CH(OH)—Me | CN | H |
| 846 | H | CC—CH(OH)—Me | H | Me |
| 847 | H | CC—CH(OH)—Me | H | Et |
| 848 | H | CC—CH(OH)—Me | H | CHF—Me |
| 849 | H | CC—CH(OH)—Me | H | i-Pr |
| 850 | H | CC—CH(OH)—Me | H | F |
| 851 | H | CC—CH(OH)—Me | H | Cl |
| 852 | H | CC—CH(OH)—Me | H | Br |
| 853 | H | CC—CH(OH)—Me | H | I |
| 854 | H | CC—CH(OH)—Me | H | NO₂ |
| 855 | H | CC—CH(OH)—Me | H | CN |
| 856 | Me | H | H | CC—CH(OH)—Me |
| 857 | Et | H | H | CC—CH(OH)—Me |
| 858 | CHF—Me | H | H | CC—CH(OH)—Me |
| 859 | i-Pr | H | H | CC—CH(OH)—Me |
| 860 | F | H | H | CC—CH(OH)—Me |
| 861 | Cl | H | H | CC—CH(OH)—Me |
| 862 | Br | H | H | CC—CH(OH)—Me |
| 863 | I | H | H | CC—CH(OH)—Me |
| 864 | NO₂ | H | H | CC—CH(OH)—Me |
| 865 | CN | H | H | CC—CH(OH)—Me |
| 866 | H | Me | CC—CH(OH)—Me | H |
| 867 | H | Et | CC—CH(OH)—Me | H |
| 868 | H | CHF—Me | CC—CH(OH)—Me | H |
| 869 | H | i-Pr | CC—CH(OH)—Me | H |
| 870 | H | F | CC—CH(OH)—Me | H |
| 871 | H | Cl | CC—CH(OH)—Me | H |
| 872 | H | Br | CC—CH(OH)—Me | H |
| 873 | H | I | CC—CH(OH)—Me | H |
| 874 | H | NO₂ | CC—CH(OH)—Me | H |
| 875 | H | CN | CC—CH(OH)—Me | H |
| 876 | H | Me | CC—CH(OH)—Me | F |
| 877 | H | Me | CC—CH(OH)—Me | Cl |
| 878 | H | Me | CC—CH(OH)—Me | Br |
| 879 | H | Me | CC—CH(OH)—Me | I |
| 880 | H | Me | CC—CH(OH)—Me | Me |
| 881 | H | Et | CC—CH(OH)—Me | Et |
| 882 | H | CHF—Me | CC—CH(OH)—Me | CHF—Me |
| 883 | H | i-Pr | CC—CH(OH)—Me | i-Pr |
| 884 | H | F | CC—CH(OH)—Me | F |
| 885 | H | Cl | CC—CH(OH)—Me | Cl |
| 886 | H | Br | CC—CH(OH)—Me | Br |
| 887 | H | I | CC—CH(OH)—Me | I |
| 888 | H | NO₂ | CC—CH(OH)—Me | NO₂ |
| 889 | H | CN | CC—CH(OH)—Me | CN |
| 890 | H | CC—CH(OH)—Me | CC—CH(OH)—Me | H |
| 891 | H | CC—CH(OH)—Me | CC—CH(OH)—Me | Me |
| 892 | H | CC—CH(OH)—Me | CC—CH(OH)—Me | Et |
| 893 | H | CC—CH(OH)—Me | CC—CH(OH)—Me | CHF—Me |
| 894 | H | CC—CH(OH)—Me | CC—CH(OH)—Me | i-Pr |
| 895 | H | CC—CH(OH)—Me | CC—CH(OH)—Me | F |
| 896 | H | CC—CH(OH)—Me | CC—CH(OH)—Me | Cl |
| 897 | H | CC—CH(OH)—Me | CC—CH(OH)—Me | Br |
| 898 | H | CC—CH(OH)—Me | CC—CH(OH)—Me | I |
| 899 | H | CC—CH(OH)—Me | CC—CH(OH)—Me | NO₂ |
| 900 | H | CC—CH(OH)—Me | CC—CH(OH)—Me | CN |
| 901 | H | CC—CH(OH)—Me | H | CC—CH(OH)—Me |
| 902 | H | CC—CH(OH)—Me | Me | CC—CH(OH)—Me |
| 903 | H | CC—CH(OH)—Me | Et | CC—CH(OH)—Me |
| 904 | H | CC—CH(OH)—Me | CHF—Me | CC—CH(OH)—Me |
| 905 | H | CC—CH(OH)—Me | i-Pr | CC—CH(OH)—Me |
| 906 | H | CC—CH(OH)—Me | F | CC—CH(OH)—Me |
| 907 | H | CC—CH(OH)—Me | Cl | CC—CH(OH)—Me |
| 908 | H | CC—CH(OH)—Me | Br | CC—CH(OH)—Me |
| 909 | H | CC—CH(OH)—Me | I | CC—CH(OH)—Me |
| 910 | H | CC—CH(OH)—Me | NO₂ | CC—CH(OH)—Me |
| 911 | H | CC—CH(OH)—Me | CN | CC—CH(OH)—Me |
| 912 | H | c-Pr | Me | H |
| 913 | H | c-Pr | Et | H |
| 914 | H | c-Pr | CHF—Me | H |
| 915 | H | c-Pr | i-Pr | H |
| 916 | H | c-Pr | F | H |
| 917 | H | c-Pr | Cl | H |
| 918 | H | c-Pr | Br | H |
| 919 | H | c-Pr | I | H |
| 920 | H | c-Pr | NO₂ | H |
| 921 | H | c-Pr | CN | H |
| 922 | H | c-Pr | H | Me |
| 923 | H | c-Pr | H | Et |
| 924 | H | c-Pr | H | CHF—Me |
| 925 | H | c-Pr | H | i-Pr |
| 926 | H | c-Pr | H | F |
| 927 | H | c-Pr | H | Cl |
| 928 | H | c-Pr | H | Br |
| 929 | H | c-Pr | H | I |
| 930 | H | c-Pr | H | NO₂ |
| 931 | H | c-Pr | H | CN |
| 932 | Me | H | H | c-Pr |
| 933 | Et | H | H | c-Pr |
| 934 | CHF—Me | H | H | c-Pr |
| 935 | i-Pr | H | H | c-Pr |
| 936 | F | H | H | c-Pr |
| 937 | Cl | H | H | c-Pr |
| 938 | Br | H | H | c-Pr |
| 939 | I | H | H | c-Pr |
| 940 | NO₂ | H | H | c-Pr |
| 941 | CN | H | H | c-Pr |
| 942 | H | Me | c-Pr | H |
| 943 | H | Et | c-Pr | H |
| 944 | H | CHF—Me | c-Pr | H |
| 945 | H | i-Pr | c-Pr | H |
| 946 | H | F | c-Pr | H |
| 947 | H | Cl | c-Pr | H |
| 948 | H | Br | c-Pr | H |
| 949 | H | I | c-Pr | H |
| 950 | H | NO₂ | c-Pr | H |
| 951 | H | CN | c-Pr | H |
| 952 | H | Me | c-Pr | F |
| 953 | H | Me | c-Pr | Cl |
| 954 | H | Me | c-Pr | Br |
| 955 | H | Me | c-Pr | I |
| 956 | H | Me | c-Pr | Me |
| 957 | H | Et | c-Pr | Et |
| 958 | H | CHF—Me | c-Pr | CHF—Me |
| 959 | H | i-Pr | c-Pr | i-Pr |
| 960 | H | F | c-Pr | F |
| 961 | H | Cl | c-Pr | Cl |
| 962 | H | Br | c-Pr | Br |
| 963 | H | I | c-Pr | I |
| 964 | H | NO₂ | c-Pr | NO₂ |
| 965 | H | CN | c-Pr | CN |
| 966 | H | c-Pr | c-Pr | H |
| 967 | H | c-Pr | c-Pr | Me |
| 968 | H | c-Pr | c-Pr | Et |
| 969 | H | c-Pr | c-Pr | CHF—Me |
| 970 | H | c-Pr | c-Pr | i-Pr |
| 971 | H | c-Pr | c-Pr | F |
| 972 | H | c-Pr | c-Pr | Cl |
| 973 | H | c-Pr | c-Pr | Br |
| 974 | H | c-Pr | c-Pr | I |
| 975 | H | c-Pr | c-Pr | NO₂ |
| 976 | H | c-Pr | c-Pr | CN |
| 977 | H | c-Pr | H | c-Pr |
| 978 | H | c-Pr | Me | c-Pr |
| 979 | H | c-Pr | Et | c-Pr |
| 980 | H | c-Pr | CHF—Me | c-Pr |
| 981 | H | c-Pr | i-Pr | c-Pr |
| 982 | H | c-Pr | F | c-Pr |
| 983 | H | c-Pr | Cl | c-Pr |
| 984 | H | c-Pr | Br | c-Pr |
| 985 | H | c-Pr | I | c-Pr |
| 986 | H | c-Pr | NO₂ | c-Pr |
| 987 | H | c-Pr | CN | c-Pr |
| 988 | H | CH(CF₂—CH₂) | Me | H |
| 989 | H | CH(CF₂—CH₂) | Et | H |
| 990 | H | CH(CF₂—CH₂) | CHF—Me | H |
| 991 | H | CH(CF₂—CH₂) | i-Pr | H |
| 992 | H | CH(CF₂—CH₂) | F | H |
| 993 | H | CH(CF₂—CH₂) | Cl | H |
| 994 | H | CH(CF₂—CH₂) | Br | H |
| 995 | H | CH(CF₂—CH₂) | I | H |
| 996 | H | CH(CF₂—CH₂) | NO₂ | H |
| 997 | H | CH(CF₂—CH₂) | CN | H |
| 998 | H | CH(CF₂—CH₂) | H | Me |
| 999 | H | CH(CF₂—CH₂) | H | Et |
| 1000 | H | CH(CF₂—CH₂) | H | CHF—Me |
| 1001 | H | CH(CF₂—CH₂) | H | i-Pr |
| 1002 | H | CH(CF₂—CH₂) | H | F |
| 1003 | H | CH(CF₂—CH₂) | H | Cl |
| 1004 | H | CH(CF₂—CH₂) | H | Br |
| 1005 | H | CH(CF₂—CH₂) | H | I |
| 1006 | H | CH(CF₂—CH₂) | H | NO₂ |
| 1007 | H | CH(CF₂—CH₂) | H | CN |
| 1008 | Me | H | H | CH(CF₂—CH₂) |
| 1009 | Et | H | H | CH(CF₂—CH₂) |
| 1010 | CHF—Me | H | H | CH(CF₂—CH₂) |
| 1011 | i-Pr | H | H | CH(CF₂—CH₂) |
| 1012 | F | H | H | CH(CF₂—CH₂) |
| 1013 | Cl | H | H | CH(CF₂—CH₂) |
| 1014 | Br | H | H | CH(CF₂—CH₂) |
| 1015 | I | H | H | CH(CF₂—CH₂) |
| 1016 | NO₂ | H | H | CH(CF₂—CH₂) |
| 1017 | CN | H | H | CH(CF₂—CH₂) |
| 1018 | H | Me | CH(CF₂—CH₂) | H |
| 1019 | H | Et | CH(CF₂—CH₂) | H |
| 1020 | H | CHF—Me | CH(CF₂—CH₂) | H |
| 1021 | H | i-Pr | CH(CF₂—CH₂) | H |
| 1022 | H | F | CH(CF₂—CH₂) | H |
| 1023 | H | Cl | CH(CF₂—CH₂) | H |
| 1024 | H | Br | CH(CF₂—CH₂) | H |
| 1025 | H | I | CH(CF₂—CH₂) | H |
| 1026 | H | NO₂ | CH(CF₂—CH₂) | H |
| 1027 | H | CN | CH(CF₂—CH₂) | H |
| 1028 | H | Me | CH(CF₂—CH₂) | F |
| 1029 | H | Me | CH(CF₂—CH₂) | Cl |
| 1030 | H | Me | CH(CF₂—CH₂) | Br |
| 1031 | H | Me | CH(CF₂—CH₂) | I |
| 1032 | H | Me | CH(CF₂—CH₂) | Me |
| 1033 | H | Et | CH(CF₂—CH₂) | Et |
| 1034 | H | CHF—Me | CH(CF₂—CH₂) | CHF—Me |
| 1035 | H | i-Pr | CH(CF₂—CH₂) | i-Pr |
| 1036 | H | F | CH(CF₂—CH₂) | F |
| 1037 | H | Cl | CH(CF₂—CH₂) | Cl |
| 1038 | H | Br | CH(CF₂—CH₂) | Br |
| 1039 | H | I | CH(CF₂—CH₂) | I |
| 1040 | H | NO₂ | CH(CF₂—CH₂) | NO₂ |
| 1041 | H | CN | CH(CF₂—CH₂) | CN |
| 1042 | H | CH(CF₂—CH₂) | CH(CF₂—CH₂) | H |
| 1043 | H | CH(CF₂—CH₂) | CH(CF₂—CH₂) | Me |
| 1044 | H | CH(CF₂—CH₂) | CH(CF₂—CH₂) | Et |
| 1045 | H | CH(CF₂—CH₂) | CH(CF₂—CH₂) | CHF—Me |
| 1046 | H | CH(CF₂—CH₂) | CH(CF₂—CH₂) | i-Pr |
| 1047 | H | CH(CF₂—CH₂) | CH(CF₂—CH₂) | F |
| 1048 | H | CH(CF₂—CH₂) | CH(CF₂—CH₂) | Cl |
| 1049 | H | CH(CF₂—CH₂) | CH(CF₂—CH₂) | Br |
| 1050 | H | CH(CF₂—CH₂) | CH(CF₂—CH₂) | I |
| 1051 | H | CH(CF₂—CH₂) | CH(CF₂—CH₂) | NO₂ |
| 1052 | H | CH(CF₂—CH₂) | CH(CF₂—CH₂) | CN |
| 1053 | H | CH(CF₂—CH₂) | H | CH(CF₂—CH₂) |
| 1054 | H | CH(CF₂—CH₂) | Me | CH(CF₂—CH₂) |
| 1055 | H | CH(CF₂—CH₂) | Et | CH(CF₂—CH₂) |
| 1056 | H | CH(CF₂—CH₂) | CHF—Me | CH(CF₂—CH₂) |
| 1057 | H | CH(CF₂—CH₂) | i-Pr | CH(CF₂—CH₂) |
| 1058 | H | CH(CF₂—CH₂) | F | CH(CF₂—CH₂) |
| 1059 | H | CH(CF₂—CH₂) | Cl | CH(CF₂—CH₂) |
| 1060 | H | CH(CF₂—CH₂) | Br | CH(CF₂—CH₂) |
| 1061 | H | CH(CF₂—CH₂) | I | CH(CF₂—CH₂) |
| 1062 | H | CH(CF₂—CH₂) | NO₂ | CH(CF₂—CH₂) |
| 1063 | H | CH(CF₂—CH₂) | CN | CH(CF₂—CH₂) |
| 1064 | H | CF(CF₂—CF₂) | Me | H |
| 1065 | H | CF(CF₂—CF₂) | Et | H |
| 1066 | H | CF(CF₂—CF₂) | CHF—Me | H |
| 1067 | H | CF(CF₂—CF₂) | i-Pr | H |
| 1068 | H | CF(CF₂—CF₂) | F | H |
| 1069 | H | CF(CF₂—CF₂) | Cl | H |
| 1070 | H | CF(CF₂—CF₂) | Br | H |
| 1071 | H | CF(CF₂—CF₂) | I | H |
| 1072 | H | CF(CF₂—CF₂) | NO₂ | H |
| 1073 | H | CF(CF₂—CF₂) | CN | H |
| 1074 | H | CF(CF₂—CF₂) | H | Me |
| 1075 | H | CF(CF₂—CF₂) | H | Et |
| 1076 | H | CF(CF₂—CF₂) | H | CHF—Me |
| 1077 | H | CF(CF₂—CF₂) | H | i-Pr |
| 1078 | H | CF(CF₂—CF₂) | H | F |
| 1079 | H | CF(CF₂—CF₂) | H | Cl |
| 1080 | H | CF(CF₂—CF₂) | H | Br |
| 1081 | H | CF(CF₂—CF₂) | H | I |
| 1082 | H | CF(CF₂—CF₂) | H | NO₂ |
| 1083 | H | CF(CF₂—CF₂) | H | CN |
| 1084 | Me | H | H | CF(CF₂—CF₂) |
| 1085 | Et | H | H | CF(CF₂—CF₂) |
| 1086 | CHF—Me | H | H | CF(CF₂—CF₂) |
| 1087 | i-Pr | H | H | CF(CF₂—CF₂) |
| 1088 | F | H | H | CF(CF₂—CF₂) |
| 1089 | Cl | H | H | CF(CF₂—CF₂) |
| 1090 | Br | H | H | CF(CF₂—CF₂) |
| 1091 | I | H | H | CF(CF₂—CF₂) |
| 1092 | NO₂ | H | H | CF(CF₂—CF₂) |
| 1093 | CN | H | H | CF(CF₂—CF₂) |
| 1094 | H | Me | CF(CF₂—CF₂) | H |
| 1095 | H | Et | CF(CF₂—CF₂) | H |
| 1096 | H | CHF—Me | CF(CF₂—CF₂) | H |
| 1097 | H | i-Pr | CF(CF₂—CF₂) | H |
| 1098 | H | F | CF(CF₂—CF₂) | H |
| 1099 | H | Cl | CF(CF₂—CF₂) | H |
| 1100 | H | Br | CF(CF₂—CF₂) | H |
| 1101 | H | I | CF(CF₂—CF₂) | H |
| 1102 | H | NO₂ | CF(CF₂—CF₂) | H |
| 1103 | H | CN | CF(CF₂—CF₂) | H |
| 1104 | H | Me | CF(CF₂—CF₂) | F |
| 1105 | H | Me | CF(CF₂—CF₂) | Cl |
| 1106 | H | Me | CF(CF₂—CF₂) | Br |
| 1107 | H | Me | CF(CF₂—CF₂) | I |
| 1108 | H | Me | CF(CF₂—CF₂) | Me |
| 1109 | H | Et | CF(CF₂—CF₂) | Et |
| 1110 | H | CHF—Me | CF(CF₂—CF₂) | CHF—Me |
| 1111 | H | i-Pr | CF(CF₂—CF₂) | i-Pr |
| 1112 | H | F | CF(CF₂—CF₂) | F |
| 1113 | H | Cl | CF(CF₂—CF₂) | Cl |
| 1114 | H | Br | CF(CF₂—CF₂) | Br |
| 1115 | H | I | CF(CF₂—CF₂) | I |
| 1116 | H | NO₂ | CF(CF₂—CF₂) | NO₂ |
| 1117 | H | CN | CF(CF₂—CF₂) | CN |
| 1118 | H | CF(CF₂—CF₂) | CF(CF₂—CF₂) | H |
| 1119 | H | CF(CF₂—CF₂) | CF(CF₂—CF₂) | Me |
| 1120 | H | CF(CF₂—CF₂) | CF(CF₂—CF₂) | Et |
| 1121 | H | CF(CF₂—CF₂) | CF(CF₂—CF₂) | CHF—Me |
| 1122 | H | CF(CF₂—CF₂) | CF(CF₂—CF₂) | i-Pr |
| 1123 | H | CF(CF₂—CF₂) | CF(CF₂—CF₂) | F |
| 1124 | H | CF(CF₂—CF₂) | CF(CF₂—CF₂) | Cl |
| 1125 | H | CF(CF₂—CF₂) | CF(CF₂—CF₂) | Br |
| 1126 | H | CF(CF₂—CF₂) | CF(CF₂—CF₂) | I |
| 1127 | H | CF(CF₂—CF₂) | CF(CF₂—CF₂) | NO₂ |
| 1128 | H | CF(CF₂—CF₂) | CF(CF₂—CF₂) | CN |
| 1129 | H | CF(CF₂—CF₂) | H | CF(CF₂—CF₂) |
| 1130 | H | CF(CF₂—CF₂) | Me | CF(CF₂—CF₂) |
| 1131 | H | CF(CF₂—CF₂) | Et | CF(CF₂—CF₂) |
| 1132 | H | CF(CF₂—CF₂) | CHF—Me | CF(CF₂—CF₂) |
| 1133 | H | CF(CF₂—CF₂) | i-Pr | CF(CF₂—CF₂) |
| 1134 | H | CF(CF₂—CF₂) | F | CF(CF₂—CF₂) |
| 1135 | H | CF(CF₂—CF₂) | Cl | CF(CF₂—CF₂) |
| 1136 | H | CF(CF₂—CF₂) | Br | CF(CF₂—CF₂) |
| 1137 | H | CF(CF₂—CF₂) | I | CF(CF₂—CF₂) |
| 1138 | H | CF(CF₂—CF₂) | NO₂ | CF(CF₂—CF₂) |
| 1139 | H | CF(CF₂—CF₂) | CN | CF(CF₂—CF₂) |
| 1140 | H | c-Bu | Me | H |
| 1141 | H | c-Bu | Et | H |
| 1142 | H | c-Bu | CHF—Me | H |
| 1143 | H | c-Bu | i-Pr | H |
| 1144 | H | c-Bu | F | H |
| 1145 | H | c-Bu | Cl | H |
| 1146 | H | c-Bu | Br | H |
| 1147 | H | c-Bu | I | H |
| 1148 | H | c-Bu | NO₂ | H |
| 1149 | H | c-Bu | CN | H |
| 1150 | H | c-Bu | H | Me |
| 1151 | H | c-Bu | H | Et |
| 1152 | H | c-Bu | H | CHF—Me |
| 1153 | H | c-Bu | H | i-Pr |
| 1154 | H | c-Bu | H | F |
| 1155 | H | c-Bu | H | Cl |
| 1156 | H | c-Bu | H | Br |
| 1157 | H | c-Bu | H | I |
| 1158 | H | c-Bu | H | NO₂ |
| 1159 | H | c-Bu | H | CN |
| 1160 | Me | H | H | c-Bu |
| 1161 | Et | H | H | C-Bu |
| 1162 | CHF—Me | H | H | c-Bu |
| 1163 | i-Pr | H | H | c-Bu |
| 1164 | F | H | H | c-Bu |
| 1165 | Cl | H | H | c-Bu |
| 1166 | Br | H | H | c-Bu |
| 1167 | I | H | H | c-Bu |
| 1168 | NO₂ | H | H | c-Bu |
| 1169 | CN | H | H | c-Bu |
| 1170 | H | Me | c-Bu | H |
| 1171 | H | Et | c-Bu | H |
| 1172 | H | CHF—Me | c-Bu | H |
| 1173 | H | i-Pr | c-Bu | H |
| 1174 | H | F | c-Bu | H |
| 1175 | H | Cl | c-Bu | H |
| 1176 | H | Br | c-Bu | H |
| 1177 | H | I | c-Bu | H |
| 1178 | H | NO₂ | c-Bu | H |
| 1179 | H | CN | c-Bu | H |
| 1180 | H | Me | c-Bu | F |
| 1181 | H | Me | c-Bu | Cl |
| 1182 | H | Me | c-Bu | Br |
| 1183 | H | Me | c-Bu | I |
| 1184 | H | Me | c-Bu | Me |
| 1185 | H | Et | c-Bu | Et |
| 1186 | H | CHF—Me | c-Bu | CHF—Me |
| 1187 | H | i-Pr | c-Bu | i-Pr |
| 1188 | H | F | c-Bu | F |
| 1189 | H | Cl | c-Bu | Cl |
| 1190 | H | Br | c-Bu | Br |
| 1191 | H | I | c-Bu | I |
| 1192 | H | NO₂ | c-Bu | NO₂ |
| 1193 | H | CN | c-Bu | CN |
| 1194 | H | c-Bu | c-Bu | H |
| 1195 | H | c-Bu | c-Bu | Me |
| 1196 | H | c-Bu | c-Bu | Et |
| 1197 | H | c-Bu | c-Bu | CHF—Me |
| 1198 | H | c-Bu | c-Bu | i-Pr |
| 1199 | H | c-Bu | c-Bu | F |
| 1200 | H | c-Bu | c-Bu | Cl |
| 1201 | H | c-Bu | c-Bu | Br |
| 1202 | H | c-Bu | c-Bu | I |
| 1203 | H | c-Bu | c-Bu | NO₂ |
| 1204 | H | c-Bu | c-Bu | CN |
| 1205 | H | c-Bu | H | c-Bu |
| 1206 | H | c-Bu | Me | c-Bu |
| 1207 | H | c-Bu | Et | c-Bu |
| 1208 | H | c-Bu | CHF—Me | c-Bu |
| 1209 | H | c-Bu | i-Pr | c-Bu |
| 1210 | H | c-Bu | F | c-Bu |
| 1211 | H | c-Bu | Cl | c-Bu |
| 1212 | H | c-Bu | Br | c-Bu |
| 1213 | H | c-Bu | I | c-Bu |
| 1214 | H | c-Bu | NO₂ | c-Bu |
| 1215 | H | c-Bu | CN | c-Bu |
| 1216 | H | c-Pen | Me | H |
| 1217 | H | c-Pen | Et | H |
| 1218 | H | c-Pen | CHF—Me | H |
| 1219 | H | c-Pen | i-Pr | H |
| 1220 | H | c-Pen | F | H |
| 1221 | H | c-Pen | Cl | H |
| 1222 | H | c-Pen | Br | H |
| 1223 | H | c-Pen | I | H |
| 1224 | H | c-Pen | NO₂ | H |
| 1225 | H | c-Pen | CN | H |
| 1226 | H | c-Pen | H | Me |
| 1227 | H | c-Pen | H | Et |
| 1228 | H | c-Pen | H | CHF—Me |
| 1229 | H | c-Pen | H | i-Pr |
| 1230 | H | c-Pen | H | F |
| 1231 | H | c-Pen | H | Cl |
| 1232 | H | c-Pen | H | Br |
| 1233 | H | c-Pen | H | I |
| 1234 | H | c-Pen | H | NO₂ |
| 1235 | H | c-Pen | H | CN |
| 1236 | Me | H | H | c-Pen |
| 1237 | Et | H | H | c-Pen |
| 1238 | CHF—Me | H | H | c-Pen |
| 1239 | i-Pr | H | H | c-Pen |
| 1240 | F | H | H | c-Pen |
| 1241 | Cl | H | H | c-Pen |
| 1242 | Br | H | H | c-Pen |
| 1243 | I | H | H | c-Pen |
| 1244 | NO₂ | H | H | c-Pen |
| 1245 | CN | H | H | c-Pen |
| 1246 | H | Me | c-Pen | H |
| 1247 | H | Et | c-Pen | H |
| 1248 | H | CHF—Me | c-Pen | H |
| 1249 | H | i-Pr | c-Pen | H |
| 1250 | H | F | c-Pen | H |
| 1251 | H | Cl | c-Pen | H |
| 1252 | H | Br | c-Pen | H |
| 1253 | H | I | c-Pen | H |
| 1254 | H | NO₂ | c-Pen | H |
| 1255 | H | CN | c-Pen | H |
| 1256 | H | Me | c-Pen | F |
| 1257 | H | Me | c-Pen | Cl |
| 1258 | H | Me | c-Pen | Br |
| 1259 | H | Me | c-Pen | I |
| 1260 | H | Me | c-Pen | Me |
| 1261 | H | Et | c-Pen | Et |
| 1262 | H | CHF—Me | c-Pen | CHF—Me |
| 1263 | H | i-Pr | c-Pen | i-Pr |
| 1264 | H | F | c-Pen | F |
| 1265 | H | Cl | c-Pen | Cl |
| 1266 | H | Br | c-Pen | Br |
| 1267 | H | I | c-Pen | I |
| 1268 | H | NO₂ | c-Pen | NO₂ |
| 1269 | H | CN | c-Pen | CN |
| 1270 | H | c-Pen | c-Pen | H |
| 1271 | H | c-Pen | c-Pen | Me |
| 1272 | H | c-Pen | c-Pen | Et |
| 1273 | H | c-Pen | c-Pen | CHF—Me |
| 1274 | H | c-Pen | c-Pen | i-Pr |
| 1275 | H | c-Pen | c-Pen | F |
| 1276 | H | c-Pen | c-Pen | Cl |
| 1277 | H | c-Pen | c-Pen | Br |
| 1278 | H | c-Pen | c-Pen | I |
| 1279 | H | c-Pen | c-Pen | NO₂ |
| 1280 | H | c-Pen | c-Pen | CN |
| 1281 | H | c-Pen | H | c-Pen |
| 1282 | H | c-Pen | Me | c-Pen |
| 1283 | H | c-Pen | Et | c-Pen |
| 1284 | H | c-Pen | CHF—Me | c-Pen |
| 1285 | H | c-Pen | i-Pr | c-Pen |
| 1286 | H | c-Pen | F | c-Pen |
| 1287 | H | c-Pen | Cl | c-Pen |
| 1288 | H | c-Pen | Br | c-Pen |
| 1289 | H | c-Pen | I | c-Pen |
| 1290 | H | c-Pen | NO₂ | c-Pen |
| 1291 | H | c-Pen | CN | c-Pen |
| 1292 | H | c-Hex | Me | H |
| 1293 | H | c-Hex | Et | H |
| 1294 | H | c-Hex | CHF—Me | H |
| 1295 | H | c-Hex | i-Pr | H |
| 1296 | H | c-Hex | F | H |
| 1297 | H | c-Hex | Cl | H |
| 1298 | H | c-Hex | Br | H |
| 1299 | H | c-Hex | I | H |
| 1300 | H | c-Hex | NO₂ | H |
| 1301 | H | c-Hex | CN | H |
| 1302 | H | c-Hex | H | Me |
| 1303 | H | c-Hex | H | Et |
| 1304 | H | c-Hex | H | CHF—Me |
| 1305 | H | c-Hex | H | i-Pr |
| 1306 | H | c-Hex | H | F |
| 1307 | H | c-Hex | H | Cl |
| 1308 | H | c-Hex | H | Br |
| 1309 | H | c-Hex | H | I |
| 1310 | H | c-Hex | H | NO₂ |
| 1311 | H | c-Hex | H | CN |
| 1312 | Me | H | H | c-Hex |
| 1313 | Et | H | H | c-Hex |
| 1314 | CHF—Me | H | H | c-Hex |
| 1315 | i-Pr | H | H | c-Hex |
| 1316 | F | H | H | c-Hex |
| 1317 | Cl | H | H | c-Hex |
| 1318 | Br | H | H | c-Hex |
| 1319 | I | H | H | c-Hex |
| 1320 | NO₂ | H | H | c-Hex |
| 1321 | CN | H | H | c-Hex |
| 1322 | H | Me | c-Hex | H |
| 1323 | H | Et | c-Hex | H |
| 1324 | H | CHF—Me | c-Hex | H |
| 1325 | H | i-Pr | c-Hex | H |
| 1326 | H | F | c-Hex | H |
| 1327 | H | Cl | c-Hex | H |
| 1328 | H | Br | c-Hex | H |
| 1329 | H | I | c-Hex | H |
| 1330 | H | NO₂ | c-Hex | H |
| 1331 | H | CN | c-Hex | H |
| 1332 | H | Me | c-Hex | F |
| 1333 | H | Me | c-Hex | Cl |
| 1334 | H | Me | c-Hex | Br |
| 1335 | H | Me | c-Hex | I |
| 1336 | H | Me | c-Hex | Me |
| 1337 | H | Et | c-Hex | Et |
| 1338 | H | CHF—Me | c-Hex | CHF—Me |
| 1339 | H | i-Pr | c-Hex | i-Pr |
| 1340 | H | F | c-Hex | F |
| 1341 | H | Cl | c-Hex | Cl |
| 1342 | H | Br | c-Hex | Br |
| 1343 | H | I | c-Hex | I |
| 1344 | H | NO₂ | c-Hex | NO₂ |
| 1345 | H | CN | c-Hex | CN |
| 1346 | H | c-Hex | c-Hex | H |
| 1347 | H | c-Hex | c-Hex | Me |
| 1348 | H | c-Hex | c-Hex | Et |
| 1349 | H | c-Hex | c-Hex | CHF—Me |
| 1350 | H | c-Hex | c-Hex | i-Pr |
| 1351 | H | c-Hex | c-Hex | F |
| 1352 | H | c-Hex | c-Hex | Cl |
| 1353 | H | c-Hex | c-Hex | Br |
| 1354 | H | c-Hex | c-Hex | I |
| 1355 | H | c-Hex | c-Hex | NO₂ |
| 1356 | H | c-Hex | c-Hex | CN |
| 1357 | H | c-Hex | H | c-Hex |
| 1358 | H | c-Hex | Me | c-Hex |
| 1359 | H | c-Hex | Et | c-Hex |
| 1360 | H | c-Hex | CHF—Me | c-Hex |
| 1361 | H | c-Hex | i-Pr | c-Hex |
| 1362 | H | c-Hex | F | c-Hex |
| 1363 | H | c-Hex | Cl | c-Hex |
| 1364 | H | c-Hex | Br | c-Hex |
| 1365 | H | c-Hex | I | c-Hex |
| 1366 | H | c-Hex | NO₂ | c-Hex |
| 1367 | H | c-Hex | CN | c-Hex |
| 1368 | H | CF═CF₂ | c-Pr | H |
| 1369 | H | CF═CF₂ | c-Pr | Me |
| 1370 | H | CF═CF₂ | c-Pr | Et |
| 1371 | H | CF═CF₂ | c-Pr | CHF—Me |
| 1372 | H | CF═CF₂ | c-Pr | i-Pr |
| 1373 | H | CF═CF₂ | c-Pr | F |
| 1374 | H | CF═CF₂ | c-Pr | Cl |
| 1375 | H | CF═CF₂ | c-Pr | Br |
| 1376 | H | CF═CF₂ | c-Pr | I |
| 1377 | H | CF═CF₂ | c-Pr | NO₂ |
| 1378 | H | CF═CF₂ | c-Pr | CN |
| 1379 | H | c-Pr | CF═CF₂ | H |
| 1380 | H | c-Pr | CF═CF₂ | Me |
| 1381 | H | c-Pr | CF═CF₂ | Et |
| 1382 | H | c-Pr | CF═CF₂ | CHF—Me |
| 1383 | H | c-Pr | CF═CF₂ | i-Pr |
| 1384 | H | c-Pr | CF═CF₂ | F |
| 1385 | H | c-Pr | CF═CF₂ | Cl |
| 1386 | H | c-Pr | CF═CF₂ | Br |
| 1387 | H | c-Pr | CF═CF₂ | I |
| 1388 | H | c-Pr | CF═CF₂ | NO₂ |
| 1389 | H | c-Pr | CF═CF₂ | CN |
| 1390 | H | c-Pr | H | CF═CF₂ |
| 1391 | H | c-Pr | Me | CF═CF₂ |
| 1392 | H | c-Pr | Et | CF═CF₂ |
| 1393 | H | c-Pr | CHF—Me | CF═CF₂ |
| 1394 | H | c-Pr | i-Pr | CF═CF₂ |
| 1395 | H | c-Pr | F | CF═CF₂ |
| 1396 | H | c-Pr | Cl | CF═CF₂ |
| 1397 | H | c-Pr | Br | CF═CF₂ |
| 1398 | H | c-Pr | I | CF═CF₂ |
| 1399 | H | c-Pr | NO₂ | CF═CF₂ |
| 1400 | H | c-Pr | CN | CF═CF₂ |
| 1401 | H | CCMe | c-Pr | H |
| 1402 | H | CCMe | c-Pr | Me |
| 1403 | H | CCMe | c-Pr | Et |
| 1404 | H | CCMe | c-Pr | CHF—Me |
| 1405 | H | CCMe | c-Pr | i-Pr |
| 1406 | H | CCMe | c-Pr | F |
| 1407 | H | CCMe | c-Pr | Cl |
| 1408 | H | CCMe | c-Pr | Br |
| 1409 | H | CCMe | c-Pr | I |
| 1410 | H | CCMe | c-Pr | NO₂ |
| 1411 | H | CCMe | c-Pr | CN |
| 1412 | H | c-Pr | CCMe | H |
| 1413 | H | c-Pr | CCMe | Me |
| 1414 | H | c-Pr | CCMe | Et |
| 1415 | H | c-Pr | CCMe | CHF—Me |
| 1416 | H | c-Pr | CCMe | i-Pr |
| 1417 | H | c-Pr | CCMe | F |
| 1418 | H | c-Pr | CCMe | Cl |
| 1419 | H | c-Pr | CCMe | Br |
| 1420 | H | c-Pr | CCMe | I |
| 1421 | H | c-Pr | CCMe | NO₂ |
| 1422 | H | c-Pr | CCMe | CN |
| 1423 | H | c-Pr | H | CCMe |
| 1424 | H | c-Pr | Me | CCMe |
| 1425 | H | c-Pr | Et | CCMe |
| 1426 | H | c-Pr | CHF—Me | CCMe |
| 1427 | H | c-Pr | i-Pr | CCMe |
| 1428 | H | c-Pr | F | CCMe |
| 1429 | H | c-Pr | Cl | CCMe |
| 1430 | H | c-Pr | Br | CCMe |
| 1431 | H | c-Pr | I | CCMe |
| 1432 | H | c-Pr | NO₂ | CCMe |
| 1433 | H | c-Pr | CN | CCMe |
| 1434 | H | CF═CF₂ | CF(CF₂—CF₂) | H |
| 1435 | H | CF═CF₂ | CF(CF₂—CF₂) | Me |
| 1436 | H | CF═CF₂ | CF(CF₂—CF₂) | Et |
| 1437 | H | CF═CF₂ | CF(CF₂—CF₂) | CHF—Me |
| 1438 | H | CF═CF₂ | CF(CF₂—CF₂) | i-Pr |
| 1439 | H | CF═CF₂ | CF(CF₂—CF₂) | F |
| 1440 | H | CF═CF₂ | CF(CF₂—CF₂) | Cl |
| 1441 | H | CF═CF₂ | CF(CF₂—CF₂) | Br |
| 1442 | H | CF═CF₂ | CF(CF₂—CF₂) | I |
| 1443 | H | CF═CF₂ | CF(CF₂—CF₂) | NO₂ |
| 1444 | H | CF═CF₂ | CF(CF₂—CF₂) | CN |
| 1445 | H | CF(CF₂—CF₂) | CF═CF₂ | H |
| 1446 | H | CF(CF₂—CF₂) | CF═CF₂ | Me |
| 1447 | H | CF(CF₂—CF₂) | CF═CF₂ | Et |
| 1448 | H | CF(CF₂—CF₂) | CF═CF₂ | CHF—Me |
| 1449 | H | CF(CF₂—CF₂) | CF═CF₂ | i-Pr |
| 1450 | H | CF(CF₂—CF₂) | CF═CF₂ | F |
| 1451 | H | CF(CF₂—CF₂) | CF═CF₂ | Cl |
| 1452 | H | CF(CF₂—CF₂) | CF═CF₂ | Br |
| 1453 | H | CF(CF₂—CF₂) | CF═CF₂ | I |
| 1454 | H | CF(CF₂—CF₂) | CF═CF₂ | NO₂ |
| 1455 | H | CF(CF₂—CF₂) | CF═CF₂ | CN |
| 1456 | H | CF(CF₂—CF₂) | H | CF═CF₂ |
| 1457 | H | CF(CF₂—CF₂) | Me | CF═CF₂ |
| 1458 | H | CF(CF₂—CF₂) | Et | CF═CF₂ |
| 1459 | H | CF(CF₂—CF₂) | CHF—Me | CF═CF₂ |
| 1460 | H | CF(CF₂—CF₂) | i-Pr | CF═CF₂ |
| 1461 | H | CF(CF₂—CF₂) | F | CF═CF₂ |
| 1462 | H | CF(CF₂—CF₂) | Cl | CF═CF₂ |
| 1463 | H | CF(CF₂—CF₂) | Br | CF═CF₂ |
| 1464 | H | CF(CF₂—CF₂) | I | CF═CF₂ |
| 1465 | H | CF(CF₂—CF₂) | NO₂ | CF═CF₂ |
| 1466 | H | CF(CF₂—CF₂) | CN | CF═CF₂ |
| 1467 | H | CCMe | CF(CF₂—CF₂) | H |
| 1468 | H | CCMe | CF(CF₂—CF₂) | Me |
| 1469 | H | CCMe | CF(CF₂—CF₂) | Et |
| 1470 | H | CCMe | CF(CF₂—CF₂) | CHF—Me |
| 1471 | H | CCMe | CF(CF₂—CF₂) | i-Pr |
| 1472 | H | CCMe | CF(CF₂—CF₂) | F |
| 1473 | H | CCMe | CF(CF₂—CF₂) | Cl |
| 1474 | H | CCMe | CF(CF₂—CF₂) | Br |
| 1475 | H | CCMe | CF(CF₂—CF₂) | I |
| 1476 | H | CCMe | CF(CF₂—CF₂) | NO₂ |
| 1477 | H | CCMe | CF(CF₂—CF₂) | CN |
| 1478 | H | CF(CF₂—CF₂) | CCMe | H |
| 1479 | H | CF(CF₂—CF₂) | CCMe | Me |
| 1480 | H | CF(CF₂—CF₂) | CCMe | Et |
| 1481 | H | CF(CF₂—CF₂) | CCMe | CHF—Me |
| 1482 | H | CF(CF₂—CF₂) | CCMe | i-Pr |
| 1483 | H | CF(CF₂—CF₂) | CCMe | F |
| 1484 | H | CF(CF₂—CF₂) | CCMe | Cl |
| 1485 | H | CF(CF₂—CF₂) | CCMe | Br |
| 1486 | H | CF(CF₂—CF₂) | CCMe | I |
| 1487 | H | CF(CF₂—CF₂) | CCMe | NO₂ |
| 1488 | H | CF(CF₂—CF₂) | CCMe | CN |
| 1489 | H | CF(CF₂—CF₂) | H | CCMe |
| 1490 | H | CF(CF₂—CF₂) | Me | CCMe |
| 1491 | H | CF(CF₂—CF₂) | Et | CCMe |
| 1492 | H | CF(CF₂—CF₂) | CHF—Me | CCMe |
| 1493 | H | CF(CF₂—CF₂) | i-Pr | CCMe |
| 1494 | H | CF(CF₂—CF₂) | F | CCMe |
| 1495 | H | CF(CF₂—CF₂) | Cl | CCMe |
| 1496 | H | CF(CF₂—CF₂) | Br | CCMe |
| 1497 | H | CF(CF₂—CF₂) | I | CCMe |
| 1498 | H | CF(CF₂—CF₂) | NO₂ | CCMe |
| 1499 | H | CF(CF₂—CF₂) | CN | CCMe |
| 1500 | H | CCH | CF(CF₂—CF₂) | H |
| 1501 | H | CCH | CF(CF₂—CF₂) | Me |
| 1502 | H | CCH | CF(CF₂—CF₂) | Et |
| 1503 | H | CCH | CF(CF₂—CF₂) | CHF—Me |
| 1504 | H | CCH | CF(CF₂—CF₂) | i-Pr |
| 1505 | H | CCH | CF(CF₂—CF₂) | F |
| 1506 | H | CCH | CF(CF₂—CF₂) | Cl |
| 1507 | H | CCH | CF(CF₂—CF₂) | Br |
| 1508 | H | CCH | CF(CF₂—CF₂) | I |
| 1509 | H | CCH | CF(CF₂—CF₂) | NO₂ |
| 1510 | H | CCH | CF(CF₂—CF₂) | CN |
| 1511 | H | CF(CF₂—CF₂) | CCH | H |
| 1512 | H | CF(CF₂—CF₂) | CCH | Me |
| 1513 | H | CF(CF₂—CF₂) | CCH | Et |
| 1514 | H | CF(CF₂—CF₂) | CCH | CHF—Me |
| 1515 | H | CF(CF₂—CF₂) | CCH | i-Pr |
| 1516 | H | CF(CF₂—CF₂) | CCH | F |
| 1517 | H | CF(CF₂—CF₂) | CCH | Cl |
| 1518 | H | CF(CF₂—CF₂) | CCH | Br |
| 1519 | H | CF(CF₂—CF₂) | CCH | I |
| 1520 | H | CF(CF₂—CF₂) | CCH | NO₂ |
| 1521 | H | CF(CF₂—CF₂) | CCH | CN |
| 1522 | H | CF(CF₂—CF₂) | H | CCH |
| 1523 | H | CF(CF₂—CF₂) | Me | CCH |
| 1524 | H | CF(CF₂—CF₂) | Et | CCH |
| 1525 | H | CF(CF₂—CF₂) | CHF—Me | CCH |
| 1526 | H | CF(CF₂—CF₂) | i-Pr | CCH |
| 1527 | H | CF(CF₂—CF₂) | F | CCH |
| 1528 | H | CF(CF₂—CF₂) | Cl | CCH |
| 1529 | H | CF(CF₂—CF₂) | Br | CCH |
| 1530 | H | CF(CF₂—CF₂) | I | CCH |
| 1531 | H | CF(CF₂—CF₂) | NO₂ | CCH |
| 1532 | H | CF(CF₂—CF₂) | CN | CCH |
| 1533 | H | CCH | c-Pr | H |
| 1534 | H | CCH | c-Pr | Me |
| 1535 | H | CCH | c-Pr | Et |
| 1536 | H | CCH | c-Pr | CHF—Me |
| 1537 | H | CCH | c-Pr | i-Pr |
| 1538 | H | CCH | c-Pr | F |
| 1539 | H | CCH | c-Pr | Cl |
| 1540 | H | CCH | c-Pr | Br |
| 1541 | H | CCH | c-Pr | I |
| 1542 | H | CCH | c-Pr | NO₂ |
| 1543 | H | CCH | c-Pr | CN |
| 1544 | H | c-Pr | CCH | H |
| 1545 | H | c-Pr | CCH | Me |
| 1546 | H | c-Pr | CCH | Et |
| 1547 | H | c-Pr | CCH | CHF—Me |
| 1548 | H | c-Pr | CCH | i-Pr |
| 1549 | H | c-Pr | CCH | F |
| 1550 | H | c-Pr | CCH | Cl |
| 1551 | H | c-Pr | CCH | Br |
| 1552 | H | c-Pr | CCH | I |
| 1553 | H | c-Pr | CCH | NO₂ |
| 1554 | H | c-Pr | CCH | CN |
| 1555 | H | c-Pr | H | CCH |
| 1556 | H | c-Pr | Me | CCH |
| 1557 | H | c-Pr | Et | CCH |
| 1558 | H | c-Pr | CHF—Me | CCH |
| 1559 | H | c-Pr | i-Pr | CCH |
| 1560 | H | c-Pr | F | CCH |
| 1561 | H | c-Pr | Cl | CCH |
| 1562 | H | c-Pr | Br | CCH |
| 1563 | H | c-Pr | I | CCH |
| 1564 | H | c-Pr | NO₂ | CCH |
| 1565 | H | c-Pr | CN | CCH |
| 1566 | CF═CF₂ | H | H | H |
| 1567 | H | CF═CF₂ | H | H |
| 1568 | H | H | CF═CF₂ | H |
| 1569 | CF═CF—CF₃ | H | H | H |
| 1570 | H | CF═CF—CF₃ | H | H |
| 1571 | H | H | CF═CF—CF₃ | H |
| 1572 | CF₂—CF═CF₂ | H | H | H |
| 1573 | H | CF₂—CF═CF₂ | H | H |
| 1574 | H | H | CF₂—CF═CF₂ | H |
| 1575 | CF₂—CF═CF— | H | H | H |
| | CF₃ | | | |
| 1576 | H | CF₂—CF═CF—CF₃ | H | H |
| 1577 | H | H | CF₂—CF═CF—CF₃ | H |
| 1578 | CCH | H | H | H |
| 1579 | H | CCH | H | H |
| 1580 | H | H | CCH | H |
| 1581 | CCMe | H | H | H |
| 1582 | H | CCMe | H | H |
| 1583 | H | H | CCMe | H |
| 1584 | CH₂CCH | H | H | H |
| 1585 | H | CH₂CCH | H | H |
| 1586 | H | H | CH₂CCH | H |
| 1587 | CH₂CCMe | H | H | H |
| 1588 | H | CH₂CCMe | H | H |
| 1589 | H | H | CH₂CCMe | H |
| 1590 | CH═C═CH₂ | H | H | H |
| 1591 | H | CH═C═CH₂ | H | H |
| 1592 | H | H | CH═C═CH₂ | H |
| 1593 | CC-Bu | H | H | H |
| 1594 | H | CC-Bu | H | H |
| 1595 | H | H | CC-Bu | H |
| 1596 | CC—CHF—Me | H | H | H |
| 1597 | H | CC—CHF—Me | H | H |
| 1598 | H | H | CC—CHF—Me | H |
| 1599 | CC—CH(OH)— | H | H | H |
| | Me | | | |
| 1600 | H | CC—CH(OH)—Me | H | H |
| 1601 | H | H | CC—CH(OH)—Me | H |
| 1602 | c-Pr | H | H | H |
| 1603 | H | c-Pr | H | H |
| 1604 | H | H | c-Pr | H |
| 1605 | CH(CF₂— | H | H | H |
| | CH₂) | | | |
| 1606 | H | CH(CF₂—CH₂) | H | H |
| 1607 | H | H | CH(CF₂—CH₂) | H |
| 1608 | CF(CF₂—CF₂) | H | H | H |
| 1609 | H | CF(CF₂—CF₂) | H | H |
| 1610 | H | H | CF(CF₂—CF₂) | H |
| 1611 | c-Bu | H | H | H |
| 1612 | H | c-Bu | H | H |
| 1613 | H | H | c-Bu | H |
| 1614 | c-Pen | H | H | H |
| 1615 | H | c-Pen | H | H |
| 1616 | H | H | c-Pen | H |
| 1617 | c-Hex | H | H | H |
| 1618 | H | c-Hex | H | H |
| 1619 | H | H | c-Hex | H |
| 1620 | H | F | CCH | H |

Table 1: Compounds of the formula (Ia)

Tr—Ar (Ia)

In the table below, the individual compounds are defined as combinations of the partial radicals Tr and Ar and also given the product numbers Px.y, x and y being numbers. A product number allows an unambiguous assignment to the structure of the compound of the formula (Tr—No.)-(Ar—No.) by the scheme:

P(No. of the radical Tr)·(No. of the radical Ar)

where the partial structures of the radicals Tr and Ar can be found in the Pretables 1.1 and 1.2 (see above). Accordingly, the product number P1.1 denotes the compound of the formula (Tr-1)-(Ar-1) or of the chemical formula (P1. 1):

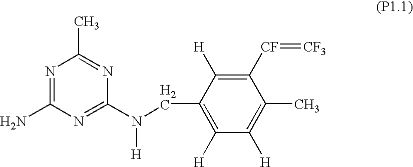

In each row, the Table 1 contains compounds of the formula (Ia) in which the radical Tr has the same partial formula (=compounds of the formula (Tr—No.)-Ar having the same radical Tr, see left-hand column). In accordance with Pretable 1.2, the radicals Ar are defined by ascending numbers. For the sake of brevity, only the first, second, penultimate and ultimate radical for Ar and the corresponding columns with product numbers are given. Accordingly, the compound of row 3 and the 5th column under Ar has the structure of the formula (Tr-3)-(Ar-5) and the product number P3.5 according to table 1.

TABLE 1

| | Ar (see definition of Ar from Table 1.2 and abbreviations) | | | | |
|---|---|---|---|---|---|
| Tr-Ar | (Ar-1) | (Ar-2) | (Ar-y) | (Ar-1619) | (Ar-1620) |
| (Tr-1)-Ar | P1.1 | P1.2 | P1.y | P1.1619 | P1.1620 |
| (Tr-2)-Ar | P2.1 | P2.2 | P2.y | P2.1619 | P2.1620 |
| (Tr-3)-Ar | P3.1 | P3.2 | P3.y | P3.1619 | P3.1620 |
| (Tr-4)-Ar | P4.1 | P4.2 | P4.y | P4.1619 | P4.1620 |
| (Tr-5)-Ar | P5.1 | P5.2 | P5.y | P5.1619 | P5.1620 |
| (Tr-6)-Ar | P6.1 | P6.2 | P6.y | P6.1619 | P6.1620 |
| (Tr-7)-Ar | P7.1 | P7.2 | P7.y | P7.1619 | P7.1620 |
| (Tr-8)-Ar | P8.1 | P8.2 | P8.y | P8.1619 | P8.1620 |
| (Tr-9)-Ar | P9.1 | P9.2 | P9.y | P9.1619 | P9.1620 |
| (Tr-10)-Ar | P10.1 | P10.2 | P10.y | P10.1619 | P10.1620 |
| (Tr-11)-Ar | P11.1 | P11.2 | P11.y | P11.1619 | P11.1620 |
| (Tr-12)-Ar | P12.1 | P12.2 | P12.y | P12.1619 | P12.1620 |
| (Tr-13)-Ar | P13.1 | P13.2 | P13.y | P13.1619 | P13.1620 |
| (Tr-14)-Ar | P14.1 | P14.2 | P14.y | P14.1619 | P14.1620 |
| (Tr-16)-Ar | P16.1 | P16.2 | P16.y | P16.1619 | P16.1620 |
| (Tr-17)-Ar | P17.1 | P17.2 | P17.y | P17.1619 | P17.1620 |
| (Tr-18)-Ar | P18.1 | P18.2 | P18.y | P18.1619 | P18.1620 |
| (Tr-19)-Ar | P19.1 | P19.2 | P19.y | P19.1619 | P19.1620 |
| (Tr-20)-Ar | P20.1 | P20.2 | P20.y | P20.1619 | P20.1620 |
| (Tr-21)-Ar | P21.1 | P21.2 | P21.y | P21.1619 | P21.1620 |
| (Tr-22)-Ar | P22.1 | P22.2 | P22.y | P22.1619 | P22.1620 |
| (Tr-23)-Ar | P23.1 | P23.2 | P23.y | P23.1619 | P23.1620 |
| (Tr-24)-Ar | P24.1 | P24.2 | P24.y | P24.1619 | P24.1620 |
| (Tr-25)-Ar | P25.1 | P25.2 | P25.y | P25.1619 | P25.1620 |
| (Tr-26)-Ar | P26.1 | P26.2 | P26.y | P26.1619 | P26.1620 |
| (Tr-27)-Ar | P27.1 | P27.2 | P27.y | P27.1619 | P27.1620 |
| (Tr-28)-Ar | P28.1 | P28.2 | P28.y | P28.1619 | P28.1620 |
| (Tr-29)-Ar | P29.1 | P29.2 | P29.y | P29.1619 | P29.1620 |
| (Tr-30)-Ar | P30.1 | P30.2 | P30.y | P30.1619 | P30.1620 |
| (Tr-31)-Ar | P31.1 | P31.2 | P31.y | P31.1619 | P31.1620 |
| (Tr-32)-Ar | P32.1 | P32.2 | P32.y | P32.1619 | P32.1620 |
| (Tr-33)-Ar | P33.1 | P33.2 | P33.y | P33.1619 | P33.1620 |
| (Tr-34)-Ar | P34.1 | P34.2 | P34.y | P34.1619 | P34.1620 |
| (Tr-35)-Ar | P35.1 | P35.2 | P35.y | P35.1619 | P35.1620 |
| (Tr-36)-Ar | P36.1 | P36.2 | P36.y | P36.1619 | P36.1620 |
| (Tr-37)-Ar | P37.1 | P37.2 | P37.y | P37.1619 | P37.1620 |
| (Tr-38)-Ar | P38.1 | P38.2 | P38.y | P38.1619 | P38.1620 |
| (Tr-39)-Ar | P39.1 | P39.2 | P39.y | P39.1619 | P39.1620 |
| (Tr-40)-Ar | P40.1 | P40.2 | P40.y | P40.1619 | P40.1620 |
| (Tr-41)-Ar | P41.1 | P41.2 | P41.y | P41.1619 | P41.1620 |
| (Tr-42)-Ar | P42.1 | P42.2 | P42.y | P42.1619 | P42.1620 |
| (Tr-43)-Ar | P43.1 | P43.2 | P43.y | P43.1619 | P43.1620 |
| (Tr-44)-Ar | P44.1 | P44.2 | P44.y | P44.1619 | P44.1620 |
| (Tr-45)-Ar | P45.1 | P45.2 | P45.y | P45.1619 | P45.1620 |
| (Tr-46)-Ar | P46.1 | P46.2 | P46.y | P46.1619 | P46.1620 |
| (Tr-47)-Ar | P47.1 | P47.2 | P47.y | P47.1619 | P47.1620 |
| (Tr-48)-Ar | P48.1 | P48.2 | P48.y | P48.1619 | P48.1620 |
| (Tr-49)-Ar | P49.1 | P49.2 | P49.y | P49.1619 | P49.1620 |
| (Tr-50)-Ar | P50.1 | P50.2 | P50.y | P50.1619 | P50.1620 |

Abbreviations:

Ar—Nr, x, y and Px.y=see definitions for tables 1, 1.1 and 1.2; y is an integer and generally runs from 1 to1620, specifically; on the 3^{rd} column under Ar it has the value 3, in the 4^{th} column it has the value 4, etc.

TABLE 2
Physical data for compounds of the formula (Ia) from Table 1

| Product (number) | Physical data |
|---|---|
| P1.5 | resin |
| P1.49 | resin |
| P1.309 | resin |
| P1.352 | resin |
| P1.384 | resin |
| P1.428 | resin |
| P1.916 | resin |
| P1.960 | resin |
| P1.1567 | resin |
| P1.1568 | resin |
| P1.1579 | resin |
| P1.1580 | resin |
| P1.1582 | resin |
| P1.1583 | resin |
| P1.1603 | resin |
| P1.1604 | resin |
| P2.5 | resin |
| P2.49 | resin |
| P2.309 | resin |
| P2.352 | resin |
| P2.384 | resin |
| P2.428 | resin |
| P2.916 | resin |
| P2.960 | resin |
| P2.1567 | resin |
| P2.1568 | resin |
| P2.1579 | resin |
| P2.1580 | resin |
| P2.1582 | resin |
| P2.1583 | resin |
| P2.1603 | resin |
| P2.1604 | resin |
| P2.1620 | resin |
| P4.5 | resin |
| P4.49 | resin |
| P4.309 | resin |
| P4.384 | resin |
| P4.428 | resin |
| P4.1567 | resin |
| P4.1568 | resin |
| P4.1579 | resin |
| P4.1580 | resin |
| P4.1582 | resin |
| P4.1583 | resin |
| P4.1603 | resin |
| P4.1604 | resin |
| P6.5 | resin |
| P6.49 | resin |
| P6.309 | resin |
| P6.352 | resin |
| P6.384 | resin |
| P6.428 | resin |
| P6.916 | resin |
| P6.960 | resin |
| P6.1567 | resin |
| P6.1568 | resin |
| P6.1579 | resin |
| P6.1580 | resin |
| P6.1582 | resin |
| P6.1583 | resin |
| P6.1603 | resin |
| P6.1604 | resin |
| P7.5 | resin |
| P7.49 | resin |
| P7.309 | oil |
| P7.335 | oil |
| P7.348 | resin |
| P7.369 | oil |
| P7.384 | oil |
| P7.428 | resin |
| P7.656 | resin |
| P7.840 | 133-138° C. |
| P7.916 | resin |
| P7.960 | resin |
| P7.1567 | resin |
| P7.1568 | resin |
| P7.1579 | resin |
| P7.1580 | oil |
| P7.1582 | resin |
| P7.1583 | resin |
| P7.1603 | resin |
| P7.1604 | oil |
| P7.1620 | resin |
| P9.309 | resin |
| P9.352 | resin |
| P9.384 | resin |
| P9.428 | resin |
| P9.1567 | resin |
| P9.1568 | resin |
| P9.1579 | resin |
| P9.1580 | resin |
| P9.1582 | resin |
| P9.1583 | resin |
| P9.1603 | resin |
| P9.1604 | resin |
| P11.1567 | resin |
| P11.1568 | resin |
| P11.1579 | resin |
| P11.1580 | resin |
| P11.1582 | resin |
| P11.1583 | resin |
| P11.1603 | resin |
| P11.1604 | resin |
| P12.5 | resin |
| P12.49 | resin |
| P12.335 | oil |
| P12.840 | oil |
| P12.916 | resin |
| P12.960 | resin |
| P12.1567 | resin |
| P12.1568 | resin |
| P12.1579 | oil |
| P12.1603 | resin |
| P12.1604 | oil |
| P12.1620 | resin |
| P14.309 | resin |
| P14.352 | resin |
| P14.384 | resin |
| P14.428 | resin |
| P14.1567 | resin |
| P14.1568 | resin |
| P14.1579 | resin |
| P14.1580 | resin |
| P14.1582 | resin |
| P14.1583 | resin |
| P14.1603 | resin |
| P14.1604 | resin |
| P16.5 | resin |
| P16.49 | resin |
| P16.309 | resin |
| P16.352 | resin |
| P16.384 | resin |
| P16.428 | resin |
| P16.916 | resin |
| P16.960 | resin |
| P16.1567 | resin |
| P16.1568 | resin |
| P16.1579 | resin |
| P16.1580 | resin |
| P16.1582 | resin |
| P16.1583 | resin |
| P16.1603 | resin |
| P16.1604 | resin |
| P17.5 | resin |
| P17.49 | resin |
| P17.309 | oil |
| P17.335 | oil |
| P17.348 | resin |
| P17.352 | 113-119° C. |
| P17.358 | 63-66° C. |
| P17.369 | oil |
| P17.384 | 121-124° C. |
| P17.424 | oil |
| P17.428 | resin |
| P17.450 | resin |
| P17.656 | resin |
| P17.688 | oil |
| P17.728 | oil |
| P17.764 | oil |
| P17.840 | oil |
| P17.916 | resin |
| P17.960 | resin |
| P17.1567 | resin |
| P17.1568 | resin |
| P17.1579 | resin |
| P17.1580 | oil |
| P17.1582 | resin |
| P17.1583 | resin |
| P17.1594 | resin |
| P17.1595 | resin |
| P17.1597 | resin |
| P17.1598 | resin |
| P17.1600 | resin |
| P17.1601 | resin |
| P17.1603 | resin |
| P17.1604 | oil |
| P17.1620 | resin |
| P19.309 | resin |
| P19.352 | resin |
| P19.384 | resin |
| P19.428 | resin |
| P19.1567 | resin |
| P19.1568 | resin |
| P19.1579 | resin |
| P19.1580 | resin |
| P19.1582 | resin |
| P19.1583 | resin |
| P19.1603 | resin |
| P19.1604 | resin |
| P21.5 | resin |
| P21.49 | resin |
| P21.309 | resin |
| P21.352 | resin |
| P21.384 | resin |
| P21.428 | resin |
| P21.916 | resin |
| P21.1567 | resin |
| P21.1568 | resin |
| P21.1579 | resin |
| P21.1580 | resin |
| P21.1582 | resin |
| P21.1583 | resin |
| P21.1603 | resin |
| P21.1604 | resin |
| P22.5 | resin |
| P22.309 | oil |
| P22.335 | oil |
| P22.348 | resin |
| P22.352 | oil |
| P22.369 | oil |
| P22.384 | oil |
| P22.428 | resin |
| P22.656 | resin |
| P22.688 | oil |
| P22.764 | resin |
| P22.840 | 119-122° C. |
| P22.916 | resin |
| P22.1567 | resin |
| P22.1568 | resin |
| P22.1579 | oil |
| P22.1580 | 134-140° C. |
| P22.1582 | resin |
| P22.1583 | resin |
| P22.1595 | resin |
| P22.1597 | resin |
| P22.1598 | resin |
| P22.1600 | resin |
| P22.1601 | resin |
| P22.1603 | resin |
| P22.1604 | oil |
| P22.1620 | resin |
| P24.1567 | resin |
| P24.1568 | resin |
| P24.1579 | resin |
| P24.1580 | resin |
| P24.1582 | resin |
| P24.1583 | resin |
| P24.1603 | resin |
| P24.1604 | resin |
| P26.5 | resin |
| P26.49 | resin |
| P26.309 | resin |
| P26.352 | resin |
| P26.384 | resin |
| P26.428 | resin |
| P26.916 | resin |
| P26.960 | resin |
| P26.1567 | resin |
| P26.1568 | resin |
| P26.1579 | resin |
| P26.1580 | resin |
| P26.1582 | resin |
| P26.1583 | resin |
| P26.1603 | resin |
| P26.1604 | resin |
| P27.5 | resin |
| P27.49 | resin |
| P27.309 | resin |
| P27.335 | resin |
| P27.352 | resin |
| P27.384 | resin |
| P27.428 | resin |
| P27.840 | oil |
| P27.916 | resin |
| P27.960 | resin |
| P27.1567 | resin |
| P27.1568 | resin |
| P27.1579 | resin |
| P27.1580 | 49-56° C. |
| P27.1582 | resin |
| P27.1583 | resin |
| P27.1600 | resin |
| P27.1601 | resin |
| P27.1603 | resin |
| P27.1604 | oil |
| P29.309 | resin |
| P29.352 | resin |
| P29.384 | resin |
| P29.428 | resin |
| P29.1567 | resin |
| P29.1568 | resin |
| P29.1579 | resin |
| P29.1580 | resin |
| P29.1582 | resin |
| P29.1583 | resin |
| P29.1603 | resin |
| P29.1604 | resin |

B. Formulation examples

a) A dust is obtained by mixing 10 parts by weight of a compound of the formula (I) and 90 parts by weight of talc as inert substance and comminuting the mixture in a hammer mill.

b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.) and grinding the mixture in a ball mill to a fineness of below 5 microns.

d) An emulsifiable concentrate is obtained from 15 parts by weight of a compound of the formula (I), 75 parts by weight of cyclohexanone as the solvent and 10 parts by weight of ethoxylated nonylphenol as the emulsifier.

e) Water-dispersible granules are obtained by mixing 75 parts by weight of a compound of the formula (I), 10 parts by weight of calcium lignosulfonate, 5 parts by weight of sodium lauryl sulfate, 3 parts by weight of polyvinyl alcohol and 7 parts by weight of kaolin grinding the mixture in a pinned-disk mill and granulating the powder in a fluidized bed by spraying on water as the granulation liquid.

f) Water-dispersible granules are also obtained by homogenizing and precomminuting, in a colloid mill, 25 parts by weight of a compound of the formula (I), 5 parts by weight of sodium 2,2′-dinaphthyAmpthane-6,6′-disulfonate 2 parts by weight of sodium oleoylmethyltaurinate, 1 part by weight of polyvinyl alcohol, 17 parts by weight of calcium carbonate and 50 parts by weight of water, subsequently grinding the mixture in a bead mill and atomizing and drying the resulting suspension in a spray tower by means of a single-substance nozzle.

C. Biological Examples

1. Pre-emergence Effect on Weeds

Seeds or rhizome pieces of monocotyledonous and dicotyledonous weed plants are placed in sandy loam soil in plastic pots and covered with soil. The compounds according to the invention which are formulated in the form of wettable powders or emulsion concentrates are then applied to the surface of the soil cover in the form of aqueous suspensions or emulsions at an application rate of 600 to 800 1 of water/ha (converted), in various dosages.

After the treatment, the pots are placed in a greenhouse and kept under good growth conditions for the weeds. After the test plants have emerged, the damage to the plants or the negative effect on the emergence is scored visually after a test period of 3 to 4 weeks by comparison with untreated controls. As shown by the test results, compounds according to the invention have good herbicidal pre-emergence activity against a broad spectrum of weed grasses and broad-leaved weeds. For example, in the test the compounds of Example Nos. P1.5, P1.49, P1.309, P1.352, P1.384, Pl.428, PI.916, PI.960, P1.1567, P1.1568, Pl.1579, Pl.1580, P1.1582, P1.1583, P1.1603, P1.1604, P2.5, P2.49, P2.309, P2.352, P2.384, P2.428, P2.916, P2.960, P2.1567, P2.1568, P2.1579, P2.1580, P2.1582, P2.1583, P2.1603, P2.1604, P2.1620, P4.5, P4.49, P4.309, P4.384, P4.428, P4.1567, P4.1568, P4.1579, P4.1580, P4.1582, P4.1583, P4.1603, P4.1604, P6.5, P6.49, P6.309, P6.352, P6.384, P6.428, P6.916, P6.960, P6.1567, P6.1568, P6.1579, P6.1580, P6.1582, P6.1583. P6.1603, P6.1604, P7.5, P7.49, P7.309, P7.335, P7.348, P7.369, P7.384, P7.428, P7.656, P7.840, P7.916, P7.960, P7.1567, P7.1568, P7.1579, P7.1580, P7.1582, P7.1583, P7.1603, P7.1604, P7.1620, P9.309, P9.352, P9.384, P9.428, P9.1567, P9.1568, P9.1579, P9.1580, P9.1582, P9.1583, P9.1603, P9.1604, P11.1567, P11.1568, P11.1579, P11.1580, P11.1582, PlI.1583, P11.1603, P11.1604, P12.5, P12.49, P12.335, P12.840, P12.916, P12.960, P12.1567, P12.1568, P12.1579, P12.1603, P12.1604, P12.1620, P14.309, P14.352, P14.384, P14.428, P14.1567, P14.1568, P14.1579, P14.1580, P14.1582, P14.1583, P14.1603, P14.1604, P16.5, P16.49, P16.309, P16.352, P16.384, P16.428, P16.916, P16.960, P16.1567, P16.1568, P16.1579, P16.1580, P16.1582, P16.1583, P16.1603, P16.1604, P17.5, P17.49, P17.309, P17.335, P17.348, P17.352, P17.358, P17.369, P17.384, P17.424, P17.428, P17.450, P17.656, P17.688, P17.728, P17.764, P17.840, P17.916, P17.960, P17.1567, P17.1568, P17.1579, P17.1580, P17.1582, P17.1583, P17.1594, P17.1595, P17.1597, P17.1598, P17.1600, P17.1601, P17.1603, P17.1604, P17.1620, P19.309, P19.352, P19.384, P19.428, P19.1567, P19.1568, P19.1579, P19.1580, P19.1582, P19.1583, P19.1603, P19.1604, P21.5, P21.49, P21.309, P21.352, P21.384, P21.428, P21.916, P21.1567, P21.1568, P21.1579, P21.1580, P21.1582, P21.1583, P21.1603, P21.1604, P22.5, P22.309, P22.335, P22.348, P22.352, P22.369, P22.384, P22.428, P22.656, P22.688, P22.764, P22.840, P22.916, P22.1567, P22.1568, P22.1579, P22.1580, P22.1582, P22.1583, P22.1595, P22.1597, P22.1598, P22.1600, P22.1601, P22.1603, P22.1604, P22.1620, P24.1567, P24.1568, P24.1579, P24.1580, P24.1582, P24.1583, P24.1603, P24.1604, P26.5, P26.49, P26.309, P26.352, P26.384, P26.428, P26.916, P26.960, P26.1567, P26.1568, P26.1579, P26.1580, P26.1582, P26.1583, P26.1603, P26.1604, P27.5, P27.49, P27.309, P27.335, P27.352, P27.384, P27.428, P27.840, P27.916, P27.960, P27.1567, P27.1568, P27.1579, P27.1580, P27.1582, P27.1583, P27.1600, P27.1601, P27.1603, P27.1604, P29.309, P29.352, P29.384, P29.428, P29.1567, P29.1568, P29.1579, P29.1580, P29.1582 (see Table 1) have very good herbicidal activity pre-emergence against harmful plants from the group consisting of Alopecurus spp., Apera spp., Poa spp., Echinochloa spp., Setaria spp., Digitaria spp., Chenopodium spp., Matricaria spp., Veronica spp., Viola spp., Stellarlia spp., s nn liCAtion rate of O5 ka and less of active substance per hectare.

2. Post-emergence Effect on Weeds

Seeds or rhizome pieces of monocotyledonous and dicotyledonous weeds are placed in sandy loam soil in plastic pots, covered with soil and grown in a greenhouse under good growth conditions. Three weeks after sowing, the test plants are treated at the three-leaf stage. The compounds according to the invention which are formulated as wettable powders or emulsion concentrates are sprayed, at various dosages, onto the green parts of the plants at an application rate of 600 to 800 1 of water/ha (converted). After the test plants have remained in the greenhouse for about 3 to 4 weeks under optimum growth conditions, the effect of the preparations is scored visually by comparison with untreated controls. The agents according to the invention also have good herbicidal activity post-emergence against a broad spectrum of economically important weed grasses and broad-leaved weeds. For example, in the test, the compounds of Example Nos. P1.5, P1.49, P1.309, P1.352, P1.384, P1.428, P1.916, P1.960, P1.1567, P1.1568, P1.1579, P1.1580, P1.1582, P1.1583, P1.1603, P1.1604, P2.5, P2.49, P2.309, P2.352, P2.384, P2.428, P2.916, P2.960, P2.1567, P2.1568, P2.1579, P2.1580, P2.1582, P2.1583, P2.1603, P2.1604, P2.1620, P4.5, P4.49, P4.309, P4.384, P4.428, P4.1567, P4.1568, P4.1579, P4.1580, P4.1582, P4.1583, P4.1603, P4.1604, P6.5, P6.49, P6.309, P6.352, P6.384, P6.428, P6.916, P6.960, P6.1567, P6.1568, P6.1579, P6.1580, P6.1582, P6.1583, P6.1603, P6.1604, P7.5, P7.49, P7.309, P7.335, P7.348, P7.369, P7.384, P7.428, P7.656, P7.840, P7.916, P7.960, P7.1567, P7.1568, P7.1579, P7.1580, P7.1582, P7.1583, P7.1603, P7.1604, P7.1620, P9.309, P9.352, P9.384, P9.428, P9.1567, P9.1568, P9.1579, P9.1580, P9.1582, P9.1583, P9.1603, P9.1604,P11.1567,P11.1568,P11.1579,P11.1580,P11.1582,P11.1583, P11.1603, P11.1604, P12.5, P12.49, P12.335, P12.840, P12.916, P12.960, P12.1567, P12.1568, P12.1579, P12.1603, P12.1604, P12.1620, P14.309, P14.352, P14.384, P14.428, P14.1567, P14.1568, P14.1579, P14.1580, P14.1582, P14.1583, P14.1603 P14.1604, P16.5, P16.49, P16.309, P16.352, P16.384, P16.428, P16.916, P16.960, P16.1567, P16.1568, P16.1579, P16.1580, P16.1582, P16.1583, P16.1603,P16.1604, P17.5, P17.49, P17.309, P17.335, P17.348, P17.352, P17.358, P17.369, P17.384, P17.424, P17.428, P17.450, P17.656, P17.688, P17.728, P17.764, P17.840, P17.916, P17.960, P17.1567, P17.1568, P17.1579, P17.1580, P17.1582, P17.1583, P17.1594, P17.1595, P17.1597, P17.1598, P17.1600, P17.1601, P17.1603, P17.1604, P17.1620, P19.309, P19.352, P19.384, P19.428, P19.1567, P19.1568, P19.1579, P19.1580, P19.1582, P19.1583, P19.1603, P19.1604, P21.5, P21.49, P21.309, P21.352, P21.384, P21.428, P21.916, P21.1567, P21.1568, P21.1579, P21.1580, P21.1582, P21.1583, P21.1603, P21.1604, P22.5, P22.309, P22.335, P22.348, P22.352, P22.369, P22.384, P22.428, P22.656, P22.688, P22.764, P22.840, P22.916, P22.1567, P22.1568, P22.1579, P22.1580, P22.1582, P22.1583, P22.1595, P22.1597, P22.1598, P22.1600, P22.1601, P22.1603, P22.1604, P22.1620, P24.1567, P24.1568, P24.1579, P24.1580, P24.1582, P24.1583, P24.1603, P24.1604, P26.5, P26.49, P26.309, P26.352, P26.384, P26.428, P26.916, P26.960, P26.1567, P26.1568, P26.1579, P26.1580, P26.1582, P26.1583, P26.1603, P26.1604, P27.5, P27.49, P27.309, P27.335, P27.352, P27.384, P27.428, P27.840, P27.916, P27.960, P27.1567, P27.1568, P27.1579, P27.1580, P27.1582, P27.1583, P27.1600, P27.1601, P27.1603, P27.1604, P29.309, P29.352, P29.384, P29.428, P29.1567, P29.1568, P29.1579, P29.1580, P29.1582 (see Table 1) have very good herbicidal activity post-emergence against harmful plants from the group consisting of Chenopodium spp., Papaver spp., Polygonum spp., Veronica spp. or Viola spp. at an application rate of 0.5 kg and less of active substance per hectare.

3. Action on Harmful Plants in Rice

Transplanted and sown rice and also typical rice weeds (gramineous and broad-leaved) are cultivated in closed plastic pots in a greenhouse to the three-leaf stage (Echinochloa crus-galli 1.5-leaf) under paddy rice conditions (dammed height of water: 2-3 cm). This is followed by treatment with the compounds according to the invention. For this purpose, the formulated active compounds are suspended, dissvlved or emulsified in water and applied by pourinn them into the dammed water around the test plants in different dosages. After this treatment, the test plants are set up in a greenhouse under optimum growth conditions and are maintained in this way throughout the test period.

About three weeks after application, evaluation is made by visual scoring of the damage to the plants by comparison with untreated controls. The compounds according to the invention show very good herbicidal activity against harmful plants. For example, the compounds of Example Nos. P1.5, P1.49, P1.309, P1.352, P1.384, P1.428, P1.916, P1.960, P1.1567, P1.1568, P1.1579, Pl.1580, P1.1582, P1.1583, P1.1603, P1.1604, P2.5, P2.49, P2.309, P2.352, P2.384, P2.428, P2.916, P2.960, P2.1567, P2.1568, P2.1579, P2.1580, P2.1582, P2.1583, P2.1603, P2.1604, P2.1620, P4.5, P4.49, P4.309, P4.384, P4.428, P4.1567, P4.1568, P4.1579, P4.1580, P4.1582, P4.1583, P4.1603, P4.1604, P6.5, P6.49, P6.309, P6.352, P6.384, P6.428, P6.916, P6.960, P6.1567, P6.1568, P6.1579, P6.1580, P6.1582, P6.1583, P6.1603, P6.1604, P7.5, P7.49, P7.309, P7.335, P7.348, P7.369, P7.384, P7.428, P7.656, P7.840, P7.916, P7.960, P7.1567, P7.1568, P7.1579, P7.1580, P7.1582, P7.1583, P7.1603, P7.1604, P7.1620, P9.309, P9.352, P9.384, P9.428, P9.1567, P9.1568, P9.1579, P9.1580, P9.1582, P9.1583, P9.1603, P9.1604, P11.1567,P11.1568,P11.1579,P11.1580,P11.1582,P11.1583,P11.1603, P11.1604, P12.5, P12.49, P12.335, P12.840, P12.916, P12.960, P12.1567, P12.1568, P12.1579, P12.1603, P12.1604, P12.1620, P14.309, P14.352, P14.384, P14.428, P14.1567, P14.1568, P14.1579, P14.1580, P14.1582, P14.1583, P14.1603, P14.1604, P16.5, P16.49, P16.309, P16.352, P16.384, P16.428, P16.916, P16.960, P16.1567, P16.1568, P16.1579, P16.1580, P16.1582, P16.1583, P16.1603, P16.1604, P17.5, P17.49, P17.309, P17.335, P17.348, P17.352, P17.358, P17.369, P17.384, P17.424, P17.428, P17.450, P17.656, P17.688, P17.728, P17.764, P17.840, P17.916, P17.960, P17.1567, P17.1568, P17.1579, P17.1580, P17.1582, P17.1583, P17.1594, P17.1595, P17.1597, P17.1598, P17.1600, P17.1601, P17.1603, P17.1604, P17.1620, P19.309, P19.352, P19.384, P19.428, P19.1567, P19.1568, P19.1579, P19.1580, P19.1582, P19.1583, P19.1603, P19.1604, P21.5, P21.49, P21.309, P21.352, P21.384, P21.428, P21.916, P21.1567, P21.1568, P21.1579, P21.1580, P21.1582, P21.1583, P21.1603, P21.1604, P22.5, P22.309, P22.335, P22.348, P22.352, P22.369, P22.384, P22.428, P22.656, P22.688, P22.764, P22.840, P22.916, P22.1567, P22.1568, P22.1579, P22.1580,P22.1582, P22.1583, P22.1595, P22.1597, P22.1598, P22.1600, P22.1601, P22.1603, P22.1604, P22.1620, P24.1567, P24.1568, P24.1579, P24.1580, P24.1582, P24.1583, P24.1603, P24.1604, P26.5, P26.49, P26.309, P26.352, P26.384, P26.428, P26.916, P26.960, P26.1567, P26.1568, P26.1579, P26.1580, P26.1582, P26.1583, P26.1603, P26.1604, P27.5, P27.49, P27.309, P27.335, P27.352, P27.384, P27.428, P27.840, P27.916, P27.960, P27.1567, P27.1568, P27.1579, P27.1580, P27.1582, P27.1583, P27.1600, P27.1601, P27.1603, P27.1604, P29.309, P29.352, P29.384, P29.428, P29.1567, P29.1568, P29.1579, P29.1580, P29.1582 (see Table 1) show very good herbicidal activity in the test against harmful plants which are typical for rice crops, for example Cyperus spp., Echinochloa spp., Digitaria spp., Sagitaria spp. and Scirpus spp.

4. Tolerance by Crop Plants

In further greenhouse experiments, seeds of a substantial number of crop plants and weeds are placed in sandy loam soil and covered with soil. Some of the pots were treated immediately as described under Section 1, and the remaining pots are placed in the greenhouse until the plants have developed two to three true leaves and then sprayed with various dosages of the substances of the formula (I) according to the invention, as described under Section 2. Visual scoring four to five weeks after the application and after the plants had been in the greenhouse reveals that compounds according to the invention left dicotyledonous crops such as soybean, cofton, oilseed rape, sugar beet or potatoes unharmed even when high dosages of active ingredient were used by the pre- and post-emergence method. Moreover, some substances also spare Gramineae crops such as barley, wheat, rye, sorghum species, corn or rice. Some of the compounds of the formula (I) have high selectivity, and they are therefore suitable for controlling undesirable vegetation in agricultural crops.

1. A compound of the formula (I) or a salt thereof, 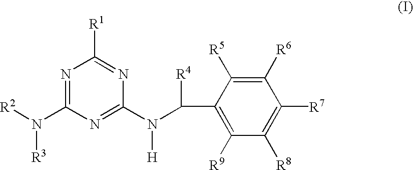
where
R¹ is (C₁-C₁₀)alkyl, (C₃-C₆)cycloalkyl, (C₁-C₁₀)alkoxy or (C₁-C₁₀)alkylthio, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl,
R² and R³ independently of one another are hydrogen, (C₁-C₄)alkyl, formyl or [(C₁-C₁₀)alkyl]carbonyl which is unsubstituted or substituted by one or more halogen atoms,
R⁴ is hydrogen, (Cl-C₁₀)alkyl, (C₂-C₁₀)alkenyl, (C₂-C₁₀)alkynyl or (C₃-C₆)cycloalkyl, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl,
each of the radicals R⁵, R⁶, R⁷, R⁸ and R⁹ independently of the others is hydrogen, halogen, nitro, cyano, thiocyanato, (C₁-C₁₀)alkyl, (C₂-C₁₀)alkenyl, (C₂-C₁₀)alkynyl, (C₁-C₁₀)alkoxy, (C₂-ClO)alkenyloxy, (C₂-C₁₀)alkynyloxy, (C₁-C₁₀)alkylthio, (C₂-C₁₀)alkenylthio, (C₂-C₁₀)alkynylthio, (C₃-C₆)cycloalkyl, (C₅-C₆)cycloalkenyl, phenyl or heterocyclyl, where each of the 13 last-mentioned radicals is unsubstituted or substituted,
where at least one of the radicals from the group consisting of R⁵, R⁶, R⁷, R⁸ and R⁹ is different from hydrogen and where at least one of the radicals R⁵, R⁶, R⁷, R⁸ and R⁹ is selected from the group consisting of the radicals (C₂-C₁₀)alkenyl, (C₂-C₁₀)alkynyl, (C₃-C₆)cycloalkyl and (C₅-C₆)cycloalkenyl, where each of the 4 last-mentioned radicals is unsubstituted or substituted.

2. A compound of the formula (I) or a salt thereof as claimed in claim 1, wherein each of the radicals R⁵, R⁶, R⁷, R⁸ and R⁹ independently of the others is hydrogen, halogen, nitro, cyano, thiocyanato, (C₁-C₁₀)alkyl, (C₂-C₁₀)alkenyl, (C₂-C₁₀)alkynyl, (C₁-C₁₀)alkoxy, (C₂-C₁₀)alkenyloxy, (C₂-C₁₀)alkynyloxy, (C₁-C₁₀)alkylthio, (C₂-C₁₀)alkenylthio, (C₂-C₁₀)alkynylthio, (C₃-C₆)cycloalkyl, (C₅-C₆)cycloalkenyl, phenyl or heterocyclyl, where each of the 13 last-mentioned radicals is unsubstituted or substituted, preferably unsubstituted or substituted by one or more radicals selected from the group consisting of halogen, cyano, hydroxy, amino, (C₁-C₆)alkoxy, (C₁-C₆)haloalkoxy and (C₁-C₆)alkylthio and, in the case of cyclic radicals, also (C₁-C₆)alkyl and (C₁-C₆)haloalkyl,
where at least one of the radicals from the group consisting of R⁵, R⁶, R⁷, R⁸ and R⁹ is different from hydrogen and where at least one of the radicals R⁵, R⁶, R⁷, R⁸ and R⁹ is selected from the group consisting of the radicals (C₂-C₁₀)alkenyl, (C₂-C₁₀)alkynyl, (C₃-C₆)cycloalkyl and (C₅-C₆)cycloalkenyl, where each of the 4 last-mentioned radicals is unsubstituted or substituted by one or more radicals selected from the group consisting of halogen, cyano, hydroxyl and amino and in the case of cyclic radicals, also (C₁-C₆)alkyl, (C₁-C₆)haloalkyl, (C₁-C₆)alkoxy, (C₁-C₆)haloalkoxy and (C₁-C₆)alkylthio.

3. A compound of the formula (I) or a salt thereof as claimed in claim 1, wherein
R¹ is (C₁-C₆)alkyl or (C₃-C₆)cycloalkyl, where each of the 2 last-mentioned radicals is unsubstituted or substituted by one or more halogen atoms,
R² and R³ independently of one another are hydrogen, formyl, methyl, ethyl or L((C₁-C₄)alkyvlcarbonyl or r(C₁-C₄)haloalkyllcarbonyl, R⁴ is hydrogen, (C₁-C₆)alkyl, (C₂-C₆)alkenyl, (C₂-C₆)alkynyl or (C₃-C₆)cycloalkyl, where each of the 4 last-mentioned radicals is unsubstituted or substituted by one or more radicals selected from the group consisting of halogen and, in the case of cyclic radicals, also (C₁-C₆)alkyl and (C₁-C₆)haloalkyl,
each of the radicals R⁵, R⁶, R⁷, R⁸ and R⁹ independently of the others is hydrogen, halogen, nitro, cyano, thiocyanato, (C₁-C₄)alkyl, (C₂-C₆)alkenyl, (C₂-C₆)alkynyl, (C₃-C₆)cycloalkyl, (C₅-C₆)cycloalkenyl, phenyl or heterocyclyl, where each of the 7 last-mentioned radicals is unsubstituted or substituted by one or more radicals selected from the group consisting of halogen, cyano and hydroxy and, in the case of cyclic radicals, also (C₁-C₄)alkyl and (C₁-C₄)haloalkyl, and at least one of the radicals from the group consisting of R⁵, R⁶, R⁷, R⁸ and R⁹ is different from hydrogen and where at least one of the radicals R⁵, R⁶, R⁷, R⁸ and R⁹ is selected from the group consisting of the radicals (C₂-C₆)alkenyl, (C₂-C₆)alkynyl, (C₃-C₆)cycloalkyl and (C₅-C₆)cycloalkenyl, where each of the 4 last-mentioned radicals is unsubstituted or substituted by one or more radicals selected from the group consisting of halogen, cyano and hydroxyl and, in the case of cyclic radicals, also (C₁-C₄)alkyl and (C₁-C₄)haloalkyl.

4. A compound of the formula (I) or a salt thereof as claimed in claim 1, wherein
R¹ is (C₁-C₄)alkyl or (C₁-C₄)haloalkyl,
one of the radicals R² and R³ is hydrogen, methyl or ethyl and the other of the radicals R² and R³ is hydrogen, formyl, methyl, ethyl or [(C₁-C₄)alkyl]carbonyl or [(C₁-C₄)haloalkyl]carbonyl,
R⁴ is hydrogen, (C₁-C₄)alkyl, (C₂-C₄)alkenyl, (C₂-C₄)alkynyl or (C₃-C₆)cycloalkyl, where each of the 4 last-mentioned radicals is unsubstituted or substituted by one or more radicals selected from the group consisting of halogen and, in the case of cyclic radicals, also (C₁-C₄)alkyl and (Cl-C₄)haloalkyl,
each of the radicals R⁵, R⁶, R⁷, R⁸ and R⁹ independently of the others is H, halogen, nitro, cyano, (Cl-C₄)alkyl, (C₂-C₆)alkenyl, (C₂-C₆)alkynyl or (C₃-C₆)cycloalkyl, where each of the 4 last-mentioned radicals is unsubstituted or substituted by one or more radicals selected from the group consisting of halogen, cyano and hydroxyl and. in the case of cyclic radicals! also (Ci-C₄)alkyl and (C₁-C₄)haloalkyl, where at least one of the radicals is different from hydrogen and at least one of the radicals different from hydrogen is (C₂-C₆)alkenyl, (C₂-C₆)alkynyl or (C₃-C₆)cycloalkyl, where each of the 3 last-mentioned radicals is unsubstituted or substituted by one or more radicals selected from the group consisting of halogen, cyano and hydroxyl and, in the case of cyclic radicals, also (C₁-C₄)alkyl and (C₁-C₄)haloalkyl.

5. A compound of the formula (I) or a salt thereof as claimed in claim 1, wherein
R¹ is (C₁-C₄)alkyl or (C₁-C₄)haloalkyl,
R² and R³ are each hydrogen,
R⁴ is hydrogen, (C₁-C₄)alkyl or (C₃-C₆)cycloalkyl, where each of the 2 last-mentioned radicals is unsubstituted or substituted by one or more radicals selected from the group consisting of halogen and, in the case of cyclic radicals, also (C₁-C₄)alkyl and (C₁-C₄)haloalkyl,
each of the radicals from the group consisting of the radicals R⁵, R⁶, R⁷, R⁸ and R⁹ independently of the others is H, halogen, nitro, cyano, (C₁-C₄)alkyl, (C₂-C₆)alkenyl, (C₂-C₆)alkynyl or (C₃-C₆)cycloalkyl, where each of the 4 last-mentioned radicals is unsubstituted or substituted, preferably unsubstituted or substituted by one or more radicals selected from the group consisting of halogen, cyano and hydroxyl and, in the case of cyclic radicals, also (C₁-C₄)alkyl and (C₁-C₄)haloalkyl, where at least one of the radicals is different from hydrogen and at least one of the radicals differing from hydrogen is (C₂-C₆)alkenyl, (C₂-C₆)alkynyl or (C₃-C₆)cycloalkyl, where each of the 3 last-mentioned radicals is unsubstituted or substituted by one or more radicals selected from the group consisting of halogen.

6. A process for preparing compounds of the formula (t) or salts thereof as defined in claim 1, which comprises
(a) reacting a compound of the formula (II),
R¹—Fu (II)
in which Fu is a functional group selected from the group consisting of carboxylic ester, carboxylic orthoester, carbonyl chloride, carboxamide, carboxylic anhydride and trichloromethyl, with a biguanidide of the formula (III) or an acid addition salt thereof 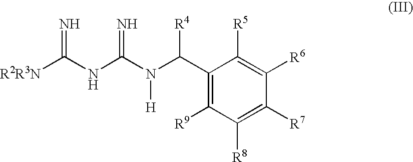
or
(b) reacting a compound of the formula (IV), 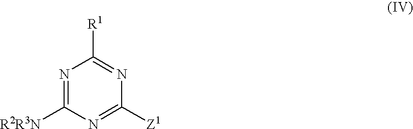
in which Z¹ is an exchangeable radical or a leaving group with a suitable amine of the formula (V) or an acid addition salt thereof 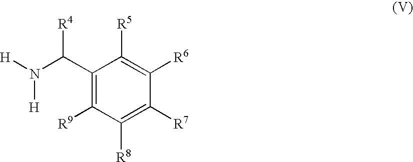
or
(c) reacting a compound of the formula (I′), 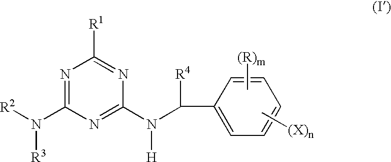
in which
X is located in the position on the phenyl ring in which in formula (I) there is a radical selected from the group of the radicals R⁵ to R⁹ representing an alkenyl, alkynyl, cycloalkyl or cycloalkenyl radical, and is a radical selected from the group consisting of halogen, trifluoromethanesulfonate radical, boronic acid group, boronic ester group and an organometalllic radical,
n is the number of these radicals and
(R)ₘ are, based on the radicals (X)ₙ the remaining radicals from the group of the radicals R⁵ to R⁹ which, with respect to the positions and the radicals, are as defined in formula (I)
with compounds of the formula R—Y, where R has the meaning of the radical in the position X on the phenyl ring defined in formula (I) and
c1) Y=hydrogen, except when R is a cycloalkyl radical, or boronic acid group, boronic ester group or an organometallic radical, in each case in the case where X is a halogen atom or a trifluoromethanesulfonate radical,
c2) Y=hydrogen, except when R is a cycloalkyl radical, or halogen or a trifluoromethanesulfonate radical, in each case in the case where X is an organometallic radical, or
c3) Y=halogen or a trifluoromethanesulfonate radical, in each case in the case where X is a boronic acid group or boronic ester group
under the conditions of the Heck reaction, Suzuki reaction, Stille reaction or Sonogashira reaction or analogous conditions to give compounds of the formula (I) or salts thereof,
where in the formulae (II), (III), (IV), (V) and (I′) the radicals R¹, R², R³, R⁴, R⁵, R⁶, R⁷, R⁸ and R⁹ are as defined in formula (I).

7. A herbicidal or plant-growth-regulating composition, which comprises at least one compound of the formula (I) or a salt thereof as claimed in claim 1 and formulation auxiliaries customary in crop protection.

8. A method for controlling harmful plants or for regulating the growth of plants, which comprises applying an effective amount of one or more compounds of the formula (I) or salts thereof as claimed in claim 1 onto the plants, seeds of plants or the area under cultivation.

9. The use of compounds of the formula (I) or salts thereof as claimed in claim 1 as herbicides and plant growth regulators.

10. The use as claimed in claim 11, wherein the compounds of the formula (I) or the salts thereof are used for controlling harmful plants or for regulating growth in crops of useful or ornamental plants.

11. A compound of the formula (III) or (V) as defined in claim 6.
